SEQUENCE LISTING (36 sequences)

SEQ ID NO: 1 - DNA, 20 bp, Artificial Sequence
  Description of Artificial Sequence synthetic oligonucleotide
gaaacgtgagggactcagca

SEQ ID NO: 2 - DNA, 26 bp, Artificial Sequence
  Description of Artificial Sequence synthetic oligonucleotide
cagcaaattatgggtcatgcggattc

SEQ ID NO: 3 - DNA, 26 bp, Artificial Sequence
  Description of Artificial Sequence synthetic oligonucleotide
cagcaagttatgagtcatgcggattc

SEQ ID NO: 4 - DNA, 26 bp, Artificial Sequence
  Description of Artificial Sequence synthetic oligonucleotide
cagcaaattatgagtcatgcggattc

SEQ ID NO: 5 - DNA, 26 bp, Artificial Sequence
  Description of Artificial Sequence synthetic oligonucleotide
cagcaagttatgggtcatgcggattc

SEQ ID NO: 6 - DNA, 20 bp, Artificial Sequence
  Description of Artificial Sequence synthetic oligonucleotide
tgctgctgctgctgctgccg

SEQ ID NO: 7 - DNA, 20 bp, Artificial Sequence
  Description of Artificial Sequence synthetic oligonucleotide
cctcctgctgctgctgctgc

SEQ ID NO: 8 - DNA, 20 bp, Artificial Sequence
  Description of Artificial Sequence synthetic oligonucleotide
ggttcctttggtatctgttt

SEQ ID NO: 9 - DNA, 20 bp, Artificial Sequence
  Description of Artificial Sequence synthetic oligonucleotide
ctccttgactgtacgccatg

SEQ ID NO: 10 - DNA, 20 bp, Artificial Sequence
  modified_base 1-4 and 17-20 are modified: Positions 1-4 and 17-20 are 2′-methoxyribose substituted nucleotides; positions 5-16 are deoxyribonucleotides
gaaacgtgagggactcagca

SEQ ID NO: 11 - DNA, 26 bp, Artificial Sequence
  modified_base 1-4 and 23-26 are modified: Positions 1-4 and 23-26 are 2′-methoxyribose substituted nucleotides; positions 5-22 are deoxyribonucleotides
cagcaaattatgggtcatgcggauuc

SEQ ID NO: 12 - DNA, 26 bp, Homo sapiens
  modified_base 1-4 and 23-26 are modified: Positions 1-4 and 23-26 are 2′-methoxyribose substituted nucleotides; positions 5-22 are deoxyribonucleotides
cagcaagttatgagtcatgcggauuc

SEQ ID NO: 13 - DNA, 26 bp, Artificial Sequence
  modified_base 1-4 and 23-26 are modified: Positions 1-4 and 23-26 are 2′-methoxyribose substituted nucleotides; positions 5-22 are deoxyribonucleotides
cagcaaattatgagtcatgcggauuc

SEQ ID NO: 14 - DNA, 26 bp, Artificial Sequence
  modified_base 1-4 and 23-26 are modified: Positions 1-4 and 23-26 are 2′-methoxyribose substituted nucleotides; positions 5-22 are deoxyribonucleotides
cagcaagttatgggtcatgcggauuc

SEQ ID NO: 15 - DNA, 20 bp, Artificial Sequence
  modified_base 1-4 and 17-20 are modified: Positions 1-4 and 17-20 are 2′-methoxyribose substituted nucleotides; positions 5-16 are deoxyribonucleotides
ugcugctgctgctgctgccg

SEQ ID NO: 16 - DNA, 20 bp, Artificial Sequence
  modified_base 1-4 and 17-20 are modified: Positions 1-4 and 17-20 are 2′-methoxyribose substituted nucleotides; positions 5-16 are deoxyribonucleotides
ccucctgctgctgctgcugc

SEQ ID NO: 17 - DNA, 20 bp, Artificial Sequence
  modified_base 1-4 and 17-20 are modified: Positions 1-4 and 17-20 are 2′-methoxyribose substituted nucleotides; positions 5-16 are deoxyribonucleotides
gguucctttggtatctguuu

SEQ ID NO: 18 - DNA, 20 bp, Artificial Sequence
  modified_base 1-4 and 17-20 are modified: Positions 1-4 and 17-20 are 2′-methoxyribose substituted nucleotides; positions 5-16 are deoxyribonucleotides
cuccttgactgtacgccaug

SEQ ID NO: 19 - DNA, 20 bp, Artificial Sequence
  modified_base 1-4 and 17-20 are modified: Positions 1-4 and 17-20 are 2′-methoxyribose substituted nucleotides; positions 5-16 are deoxyribonucleotides
caaucgtcagagactccgaa

SEQ ID NO: 20 - DNA, 26 bp, Artificial Sequence
  modified_base 1-4 and 23-26 are modified: Positions 1-4 and 23-26 are 2′-methoxyribose substituted nucleotides; positions 5-22 are deoxyribonucleotides
aaggaagtcatgaatgatgcccauug

SEQ ID NO: 21 - DNA, 26 bp, Artificial Sequence
  modified_base 1-4 and 23-26 are modified: Positions 1-4 and 23-26 are 2′-methoxyribose substituted nucleotides; positions 5-22 are deoxyribonucleotides
aaggaaatcatggatgatgcccauug

SEQ ID NO: 22 - DNA, 26 bp, Artificial Sequence
  modified_base 1-4 and 23-26 are modified: Positions 1-4 and 23-26 are 2′-methoxyribose substituted nucleotides; positions 5-22 are deoxyribonucleotides
aaggaagtcatggatgatgcccattg

SEQ ID NO: 23 - DNA, 26 bp, Artificial Sequence
  modified_base 1-4 and 23-26 are modified: Positions 1-4 and 23-26 are 2′-methoxyribose substituted nucleotides; positions 5-22 are deoxyribonucleotides
aaggaaatcatgaatgatgcccattg

SEQ ID NO: 24 - protein, 482 aa, Homo sapiens
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 25 - DNA, 1611 bp, Homo sapiens
atgtctggggtctctgcccgctggtgctgctgtctcccactcggtcatcctgagaacacagcctgagcgtctctgtcactcggggtagaccacgcggggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttctttcccc

SEQ ID NO: 26 - protein, 488 aa, Homo sapiens
MAYSQGGGKKKVCYYYDGDIGNYYYGQGHPMKPHRIRMTHNLLLNYGLYRKMEIYRPHKATAEEMTKYHSDEYIKFLRSIRPDNMSEYSKQMHIFNVGEDCPAFDGLFEFCQLSTGGSVAGAVKLNRQQTDMAVNWAGGLHHAKKYEASGFCYVNDIVLAILELLKYHQRVLYIDIDIHHGDGVEEAFYTTDRVMTVSFHKYGEYFPGTGDLRDIGAGKGKYYAVNFPMCDGIDDESYGQIFKPIISKVMEMYQPSAVVLQCGADSLSGDRLGCFNLTVKGHAKCVEVVKTFNLPLLMLGGGGYTIRNVARCWTYETAVALDCEIPNELPYNDYFEYFGPDFKLHISPSNMTNQNTPEYMEKIKQRLFENLRMLPHAPGVQMQAIPEDAVHEDSGDEDGEDPDKRISIRASDKRIACDEEFSDSEDEGEGGRRNVADHKKGAKKARIEEDKKETEDKKTDVKEEDKSKDNSGEKTDTKGTKSEQLSNP

SEQ ID NO: 27 - DNA, 1985 bp, Homo sapiens
cgccgagctttcggcacctctgccgggtggtaccgagccttcccggcgccccctcctctcctcccaccggcctgcccttccccgcgggactatcgcccccacgtttccctcagcccttttctctcccggccgagccgcggcggcagcagcagcagcagcagcagcaggaggaggagcccggtggcggcggtggccggggagcccatggcgtacagtcaaggaggcggcaaaaaaaaagtctgctactactacgacggtgatattggaaattattattatggacagggtcatcccatgaagcctcatagaatccgcatgacccataacttgctgttaaattatggcttatacagaaaaatggaaatatataggccccataaagccactgccgaagaaatgacaaaatatcacagtgatgagtatatcaaatttctacggtcaataagaccagataacatgtctgagtatagtaagcagatgcatatatttaatgttggagaagattgtccagcgtttgatggactctttgagttttgtcagctctcaactggcggttcagttgctggagctgtgaagttaaaccgacaacagactgatatggctgttaattgggctggaggattacatcatgctaagaaatacgaagcatcaggattctgttacgttaatgatattgtgcttgccatccttgaattactaaagtatcatcagagagtcttatatattgatatagatattcatcatggtgatggtgttgaagaagctttttatacaacagatcgtgtaatgacggtatcattccataaatatggggaatactttcctggcacaggagacttgagggatattggtgctggaaaaggcaaatactatgctgtcaattttccaatgtgtgatggtatagatgatgagtcatatgggcagatatttaagcctattatctcaaaggtgatggagatgtatcaacctagtgctgtggtattacagtgtggtgcagactcattatctggtgatagactgggttgtttcaatctaacagtcaaaggtcatgctaaatgtgtagaagttgtaaaaacttttaacttaccattactgatgcttggaggaggtggctacacaatccgtaatgttgctcgatgttggacatatgagactgcagttgcccttgattgtgagattcccaatgagttgccatataatgattactttgagtattttggaccagacttcaaactgcatattagtccttcaaacatgacaaaccagaacactccagaatatatggaaaagataaaacagcgtttgtttgaaaatttgcgcatgttacctcatgcacctggtgtccagatgcaagctattccagaagatgctgttcatgaagacagtggagatgaagatggagaagatccagacaagagaatttctattcgagcatcagacaagcggatagcttgtgatgaagaattctcagattctgaggatgaaggagaaggaggtcgaagaaatgtggctgatcataagaaaggagcaaagaaagctagaattgaagaagataagaaagaaacagaggacaaaaaaacagacgttaaggaagaagataaatccaaggacaacagtggtgaaaaaacagataccaaaggaaccaaatcagaacagctcagcaacccctgaatttgacagtctcaccaatttcagaaaatcattaaaaagaaaatattgaaaggaaaatgttttctttttgaagacttctggcttcattttatactactttggcatggactgtatttattttcaaatgggactttttcgtttttgtttttctgggcaagttttattgtgagattttctaattatgaagcaaaatttcttttctccaccatgctttatgtgatagtatttaaaattgatgtgagttattatgtcaaaaaaactgatctattaaagaagtaattggcctttctgagctgaaaaaaaaaaaaaaaaag

SEQ ID NO: 28 - protein, 428 aa, Homo sapiens
MAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKICDIAINWAGGLHHAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQIRQTIFENLKMLNHAPSVQIHDVPADLLTYDRTDEADAEERGPEENYSRPEAPNEFYDGDHDNDKESDVEI

SEQ ID NO: 29 - DNA, 1954 bp, Homo sapiens
ggaattcgcggccgcggcgggcgcgggaggtgcggggcctgctcccgccggcaccatggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaagagtggcttgggatgctgtgtcccaaggaatttcttttcacctcttggaagggctggagggaaaaggagtggctcctagagtcctgggggtcaccccaggggcttttgctgactctgggaaagagtctggagaccacatttggttctcgaaccatctacctgcttttcctctctctcccaaggactgacaatggtacctattagggatgagatacagacaaggatagctatctgggacattattggcagtgggccctggaggcagtccctagccccccttgccccttatttcttccctgcttccctcgaacccagagatttttgagggatgaacgggtagacaaggactgagattgcctctgacttcctcctcccctgggttctgaccttcttcctccccttgcttccagggaagatgaagagagagagatttggaaggggctctggctccctaacacctgaatcccagatgatgggaagtatgttttcaagtgtggggaggatatgaaaatgttctgttctcacttttggctttatgtccattttaccactgtttttatccaataaactaagtcggtattttttgtacctttgatggtttagcggccgcgc

SEQ ID NO: 30 - protein, 967 aa, Homo sapiens
MLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 31 - DNA, 8459 bp, Homo sapiens
ggaggttgtggggccgccgccgcggagcaccgtccccgccgccgcccgagcccgagcccgagcccgcgcacccgcccgcgccgccgccgccgccgcccgaacagcctcccagcctgggcccccggcggcgccgtggccgcgtcccggctgtcgccgcccgagcccgagcccgcgcgccggcgggtggcggcgcaggctgaggagatgcggcgcggagcgccggagcagggctagagccggccgccgccgcccgccgcggtaagcgcagccccggcccggcgcccgcgggccattgtccgccgcccgccccgcgccccgcgcagcctgcaggccttggagcccgcggcaggtggacgccgccggtccacacccgccccgcgcgcggccgtgggaggcgggggccagcgctggccgcgcgccgtgggacccgccggtccccagggccgcccggccccttctggacctttccacccgcgccgcgaggcggcttcgcccgccggggcgggggcgcgggggtgggcacggcaggcagcggcgccgtctcccggtgcggggcccgcgccccccgagcaggttcatctgcagaagccagcggacgcctctgttcaacttgtgggttacctggctcatgagaccttgccggcgaggctcggcgcttgaacgtctgtgacccagccctcaccgtcccggtacttgtatgtgttggtgggagtttggagctcgttggagctatcgtttccgtggaaattttgagccatttcgaatcacttaaaggagtggacattgctagcaatgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaataacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtagcactccctcgaagctgctgttctcttgtctgtctgtctctgtcttgaagctcagccaagaaactttcccgtgtcacgcctgcgtcccaccgtggggctctcttggagcacccagggacacccagcgtgcaacagccacgggaagcctttctgccgcccaggcccacaggtctcgagacgcacatgcacgcctgggcgtggcagcctcacagggaacacgggacagacgccggcgacgcgcagacacacggacacgcggaagccaagcacactctggcgggtcccgcaagggacgccgtggaagaaaggagcctgtggcaacaggcggccgagctgccgaattcagttgacacgaggcacagaaaacaaatatcaaagatctaataatacaaaacaaacttgattaaaactggtgcttaaagtttattacccacaactccacagtctctgtgtaaaccactcgactcatcttgtagcttattttttttttaaagaggacgttttctacggctgtggcccgcctctgtgaaccatagcggtgtgcggcggggggtctgcacccgggtgggggacagagggacctttaaagaaaacaaaactggacagaaacaggaatgtgagctgggggagctggcttgagtttctcaaaagccatcggaagatgcgagtttgtgcctttttttttattgctctggtggatttttgtggctgggttttctgaagtctgaggaacaatgccttaagaaaaaacaaacagcaggaatcggtgggacagtttcctgtggccagccgagcctggcagtgctggcaccgcgagctggcctgacgcctcaagcacgggcaccagccgtcatctccggggccaggggctgcagcccggcggtccctgttttgctttattgctgtttaagaaaaatggaggtagttccaaaaaagtggcaaatcccgttggaggttttgaagtccaacaaattttaaacgaatccaaagtgttctcacacgtcacatacgattgagcatctccatctggtcgtgaagcatgtggtaggcacacttgcagtgttacgatcggaatgctttttattaaaagcaagtagcatgaagtattgcttaaattttaggtataaataaatatatatatgtataatatatattccaatgtattccaagctaagaaacttacttgattcttatgaaatcttgataaaatatttataatgcatttatagaaaaagtatatatatatatataaaatgaatgcagattgcgaaggtccctgcaaatggatggcttgtgaatttgctctcaaggtgcttatggaaagggatcctgattgattgaaattcatgttttctcaagctccagattggctagatttcagatcgccaacacattcgccactgggcaactaccctacaagtttgtactttcattttaattattttctaacagaaccgctcccgtctccaagccttcatgcacatatgtacctaatgagtttttatagcaaagaatataaatttgctgttgatttttgtatgaattttttcacaaaaagatcctgaataagcattgttttatgaattttacatttttcctcaccatttagcaattttctgaatggtaataatgtctaaatctttttcctttctgaattcttgcttgtacatttttttttacctttcaaaggtttttaattatttttgtttttatttttgtacgatgagttttctgcagcgtacagaattgttgctgtcagattctattttcagaaagtgagaggagggaccgtaggtcttttcggagtgacaccaacgattgtgtctttcctggtctgtcctaggagctgtataaagaagcccaggggctctttttaactttcaacactagtagtattacgaggggtggtgtgtttttcccctccgtggcaagggcagggagggttgcttaggatgcccggccaccctgggaggcttgccagatgccgggggcagtcagcattaatgaaactcatgtttaaacttctctgaccacatcgtcaggatagaattctaacttgagttttccaaagaccttttgagcatgtcagcaatgcatggggcacacgtggggctctttacccacttgggtttttccactgcagccacgtggccagccctggattttggagcctgtggctgcaaggaacccagggacccttgttgcctggtgaacctgcagggagggtatgattgcctgaccaggacagccagtctttactctttttctcttcaacagtaactgacagtcacgttttactggtaacttattttccagcacatgaagccaccagtttcattccaaagtgtatattgggttcagacttgggggcagaagttcagacacaccgtgctcaggagggacccagagccgagtttcggagtttggtaaagtttacagggtagcttctgaaattaactcaaacttttgaccaaatgagtgcagattcttggattcacttggtcactgggctgctgatggtcagctctgagacagtggtttgagagcaggcagaacggtcttgggacttgtttgactttcccctccctggtggccactctttgctctgaagcccagattggcaagaggagctggtccattccccattcatggcacagagcagtggcagggcccagctagcaggctcttctggcctccttggcctcattctctgcatagccctctggggatcctgccacctgccctcttaccccgccgtggcttatggggaggaatgcatcatctcactttttttttttaagcagatgatgggataacatggactgctcagtggccaggttatcagtggggggacttaattctaatctcattcaaatggagacgccctctgcaaaggcctggcagggggaggcacgtttcatctgtcagctcactccagcttcacaaatgtgctgagagcattactgtgtagccttttctttgaagacacactcggctcttctccacagcaagcgtccagggcagatggcagaggatctgcctcggcgtctgcaggcgggaccacgtcagggagggttccttcatgtgttctccctgtgggtccttggacctttagcctttttcttcctttgcaaaggccttgggggcactggctgggagtcagcaagcgagcactttatatccctttgagggaaaccctgatgacgccactgggcctcttggcgtctgccctgccctcgcggcttcccgccgtgccgcagcgtgcccacgtgcccacgccccaccagcaggcggctgtcccggaggccgtggcccgctgggactggccgcccctccccagcgtcccagggctctggttctggagggccactttgtcaaggtgtttcagtttttctttacttcttttgaaaatctgtttgcaaggggaaggaccatttcgtaatggtctgacacaaaagcaagtttgatttttgcagcactagcaatggactttgttgtttttctttttgatcagaacattccttctttactggtcacagccacgtgctcattccattcttctttttgtagactttgggcccacgtgttttatgggcattgatacatatataaatatatagatataaatatatatgaatatatttttttaagtttcctacacctggaggttgcatggactgtacgaccggcatgactttatattgtatacagattttgcacgccaaactcggcagctttggggaagaagaaaaatgcctttctgttcccctctcatgacatttgcagatacaaaagatggaaatttttctgtaaaacaaaaccttgaaggagaggagggcggggaagtttgcgtcttattgaacttattcttaagaaattgtactttttattgtaagaaaaataaaaaggactacttaaacatttgtcatattaagaaaaaaagtttatctagcacttgtgacataccaataatagagtttattgtatttatgtggaaacagtgttttagggaaactactcagaattcacagtgaactgcctgtctctctcgagttgatttggaggaattttgttttgttttgttttgtttgtttccttttatctccttccacgggccaggcgagcgccgcccgccctcactggccttgtgacggtttattctgattgagaactgggcggactcgaaagagtccccttttccgcacagctgtgttgactttttaattacttttaggtgatgtatggctaagatttcactttaagcagtcgtgaactgtgcgagcactgtggtttacaattatactttgcatcgaaaggaaaccatttcttcattgtaacgaagctgagcgtgttcttagctcggcctcactttgtctctggcattgattaaaagtctgctattgaaagaaaaag

SEQ ID NO: 32 - protein, 716 aa, Homo sapiens
LRQGGTLTGKFMSTSSIPGCLLGVALEGDGSPHGHASLLQHVLLLEQARQQSTLIAVPLHGQSPLVTGERVATSMRTVGKLPRHRPLSRTQSSPLPQSPQALQQLVMQQQHQQFLEKQKQQQLQLGKILTKTGELPRQPTTHPEETEEELTEQQEVLLGEGALTMPREGSTESESTQEDLEEEDEEEDGEEEEDCIQVKDEEGESGAEEGPDLEEPGAGYKKLFSDAQPLQPLQVYQAPLSLATVPHQALGRTQSSPAAPGGMKSPPDQPVKHLFTTGVVYDTFMLKHQCMCGNTHVHPEHAGRIQSIWSRLQETGLLSKCERIRGRKATLDEIQTVHSEYHTLLYGTSPLNRQKLDSKKLLGPISQKMYAVLPCGGIGVDSDTVWNEMHSSSAVRMAVGCLLELAFKVAAGELKNGFAIIRPPGHHAEESTAMGFCFFNSVAITAKLLQQKLNVGKVLIVDWDIHHGNGTQQAFYNDPSVLYISLHRYDNGNFFPGSGAPEEVGGGPGVGYNVNVAWTGGVDPPIGDVEYLTAFRTVVMPIAHEFSPDVVLVSAGFDAVEGHLSPLGGYSVTARCFGHLTRQLMTLAGGRVVLALEGGHDLTAICDASEACVSALLSVELQPLDEAVLQQKPNINAVATLEKVIEIQSKHWSCVQKFAAGLGRSLREAQAGETEEAETVSAMALLSVGAEQAQAAAAREHSPRPAEEPMEQEPAL

SEQ ID NO: 33 - DNA, 2233 bp, Homo sapiens
ccctgcggcagggtggcacgctgaccggcaagttcatgagcacatcctctattcctggctgcctgctgggcgtggcactggagggcgacgggagcccccacgggcatgcctccctgctgcagcatgtgctgttgctggagcaggcccggcagcagagcaccctcattgctgtgccactccacgggcagtccccactagtgacgggtgaacgtgtggccaccagcatgcggacggtaggcaagctcccgcggcatcggcccctgagccgcactcagtcctcaccgctgccgcagagtccccaggccctgcagcagctggtcatgcaacaacagcaccagcagttcctggagaagcagaagcagcagcagctacagctgggcaagatcctcaccaagacaggggagctgcccaggcagcccaccacccaccctgaggagacagaggaggagctgacggagcagcaggaggtcttgctgggggagggagccctgaccatgccccgggagggctccacagagagtgagagcacacaggaagacctggaggaggaggacgaggaagaggatggggaggaggaggaggattgcatccaggttaaggacgaggagggcgagagtggtgctgaggaggggcccgacttggaggagcctggtgctggatacaaaaaactgttctcagatgcccagccgctgcagcctttgcaggtgtaccaggcgcccctcagcctggccactgtgccccaccaggccctgggccgtacccagtcctcccctgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctatgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgcatggcagtgggctgcctgctggagctggccttcaaggtggctgcaggagagctcaagaatggatttgccatcatccggcccccaggacaccacgccgaggaatccacagccatgggattctgcttcttcaactctgtagccatcaccgcaaaactcctacagcagaagttgaacgtgggcaaggtcctcatcgtggactgggacattcaccatggcaatggcacccagcaggcgttctataatgacccctctgtgctctacatctctctgcatcgctatgacaacgggaacttctttccaggctctggggctcctgaagaggttggtggaggaccaggcgtggggtacaatgtgaacgtggcatggacaggaggtgtggacccccccattggagacgtggagtaccttacagccttcaggacagtggtgatgcccattgcccacgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgagaccgaggaggccgagactgtgagcgccatggccttgctgtcggtgggggccgagcaggcccaggctgcggcagcccgggaacacagccccaggccggcagaggagcccatggagcaggagcctgccctgtgacgccccggcccccatccctctgggcttcaccattgtgattttgtttattttttctattaaaaacaaaaagtcacacattc

SEQ ID NO: 34 - protein, 112 aa, Homo sapiens
TIVKPVAKEFDPDMVLVSAGFDALEGHTPPLGGYKVTAKCFGHLTKQLMTLADGRVVLALEGGHDLTAICDASEACVNALLGNELEPLAEDILHQSPNMNAVISLQKIIEIQ

SEQ ID NO: 35 - DNA, 80331 bp, Homo sapiens
ttaaagacatactttgaatttcaatgatcttctgtaaagaaataacagcattcatattcgggctttggtggagaatatcttctgcaagtggctccagctgcaagaatagtagataatagtatgagcagaatattatgactttatagtaagtctcacagaagcaaagcaaactgaaacaacctaatcctcaggaaaagcttgctccgagaactagttaacattttgctgatattagttctagaagtgtagccagttttattttctgttttgtatccatgtaattgaattccaaggggcttttccattggtcacaagactaatctatgaagtccctgaagcaggtgcatgtctgtgcattattcattaaaaatctgagttatttgtgtaatttttgtaggttcctgacaaaccaaattccatcatgtctgtatatgcagctgggccctgcaaacaaaacctcagggaagttgcaatagacagaaagtgggcactctattatccatactttcacagtgtttttacatccaaccaccttatacatttgaacacttcacgatagaaagccatgagacacagctgcttttattcaaaggccattgaataaagtagtagagaaagggttcaaataaccaaatgtaaaataactgtacagtttcatttaacttctactttattcataactatgcagatgtctgtgtgtaagcacaggccaaactcctaccttgcaaatctatcataatcttctggaattacactaatttttcatggagaagctcagcaagcattgctttgcaagaaatgaagagaagctttaaataatttacagagcctatctgttaggcctaagtctgataacacatccctcagaatgctgtggtgattagtttggcatgttgcagcagaaagcgcatggctagggcccttgcaaataaaatagttgtccagctattgtgacccaaaaagcaaataaagtaatagtaacaaaaggattaacaaaaaagtagttttcctagaaacattaaggttaatcatctcaaataagaactggtgatcaaactataatatggcacgaatgggaagcgatgtcactagacgcatcaaagcatgaactcatctatgtctcttcatctgtaggaaaacctaaaaaaacacagggattaccagtttccaccacatatgtgagtgaatcttcccctcagggtccctctgaattgattattctattcttcaatcatgaaatctacaacaatagcacctttattccagatgccacaggataacagactagctcagggatatttatacacgggtaaattcactaactataactttccagagtgtttttcttagctgccaaagaaaaacttttaaacatcaaaaggtttatttgcaatacagtttggtgagaggaaaaaaaaaaaaaaacatcaccaccaccgctctgtggcagtacaaggttctgaaataaactcttaaaacttgttaaaatcattttttaaaatgtggtatttcaaattgtatcttcaagaatatgttagtctagtaacactctcttaactcagaggaactactgaatcataaactgaagcttacttggaaaactcttatgcattagacaaggttcagagggctactttgcttataaataatttttcactcctttcttggagcttagagaaaataatttttaatagaaatcaggtagattttactaataaaatatcttgaaatgacactctatgttccaatatttaagaacaaaagcacaaagcgatgacagcttatttagctcttcaaaaagcttccctgtgactttggggtaaaaatcctgacaaactgaaaggtgctggctcctggaaagaaagctataggcagatcatatatgtaaaacatctaacttctggtctcttgcacactgaggtctgggctattttaaagactcaggttatacagaaagcatacgtgattcagagtattgagagtaaaatacaaacacccctaaacccccaatttagaatcttcagttagatgcaaaagggttgtgtctattttaaaaagcaattcagagaattaaggcaattaaattgatcatttaaacctcccaaaccatttccctgcatgttttagttgaatgtcatttagaggatttaaccttcagtaaattgccccaatttgtttcattttgagtgtatacatacagacagagctgaattcagaaatttctcaatgtaggttctttattttttctctggcaggtatgcaatggataatattctctccgttggcaagaaggcaagtggcatttagagtctgtttttataaaattaaaatcttgtatggcgggaccacagaactggattgtcttttctagacatttatttaatcactcacaattataggcactaacaacatctgattgaagcatccacttaataaatatttatgagtgttgtcattattcataatttaataactaataaaataatattaaatattcattttttataaatttggagcagaaaataagaatatgtggtaaagttttgctatgtatttaatttaggtgattagatacagcaaaaacattggtgatggagattaatcagcattactggaaattattaaaagataaataaggagtaatttatacaaacaagacaacttttgcatgcaaaagttacagaaagatgaagcagatgcaaatgtaggaaatagtacacacccgtcatgccatttctgtcaagcaattctgttaaataatggtaattatcatggagttataagatacactagataattttaacacaacctcttgacacttaaagctctcatccagcttaattgtatctaaagcttaatcacaagcattactcatagcaattctttacacagattttcaggctcagtgacagggaaatttatttttctacatcattttcagaaagttgtgataatgtattggcatgattcagatgtttgttaagttgcatataccatatacattctaagtactgcagctattttaaaacaccataaaattgtggccattatcatcttataagtaatttccaggttctttagaatcagatcatttaaatcgtcaaaaatcattttagttgcctaagattcatctatagaaagaggcgagggatattcttggagaagctgttgagtaagatttttaaaatttacgctgtactgatgagctatgaaaaacagctcactgattttttttatttgcatgacttagaacagaacataaaagaagctaaacagagcctttgcaaatgtaacaggtgtgtgaggatggatgtatacatcaatcaagcaaaggatctattaaatagacttgttagcgtttttgttatgtgttgacgtttcccaaatatgccaagtattttaatatttctgtacttttatctattctgtttcctctgaattctatttttttcattcttctttgaattggtaaaaccttaccaagcattcaaggaccaccgcaaatattgcgtgctcctcaaattctccactagagcaaatttcttcccctcctatggccctttcccccacaaatattcttatttactaaatacattatgtagtagaaagtcatttttatcccactctctattttgttgtgagccatttaaagacgaagatggagactgtatatgaaagacattttaaaaatgttgaataaatggagaaatacttaatcgaaaaaataacatgaagaggtggcctttattctgctcaccatagctaacaagacaaaataaaatgggttcttcctcttggtttcctgctgtaatcaggatatggttttaagatgaagaattagaacagcagtgataactcattttgttttctaaggtgaactgctcttgcataatttccacacaattattttctcctatgcattggcaaggtttcctgacatgcaggcagatatttctatagatcagaaatgcagggaaatgcaaaagaaacaaagacccagggcaagtagaggcagaatggccatgccaataattagaggagaaaaggttgcttccaattgagcggaagaaaaagcaatagaatgacaataaaatggaatgaaattttgaaaggcattgaaattaatctttaatccaatttgaggagaactgggatcatctcaatacctagacctctaatccataacataatgtattctataatttatttagatgtttgttaacttctctaaaaatattctacaattttatgaatagggtcatattttcagatagatttattctaggtatttggtttgtttaatgcttttgtaaattatgtatgtgtatatatttacactatatatataaaatacgtatatttataatttaattaaaaaattttcaatcctggtgtgattctgtcctgtgtgtttgttctctggagtaggagtcatttatctccattcgccttctctcccaccacagtgggtgccacaaacccatagaagatttgatggacgtggacatgagccctctaaggcctcaaaattaactttttaattgtgaactaaagctgacaaatattatcactttaatgtggatgatgatgaaaatgagcaccagttttctttaagaatggccagttcaggagctggtacctatgatgaattgcacatcgttgaagcagaagcaatgaaatataaacgcagtccaaataaaagtaacactggcaactttgaaaatgtctgtacagccaacagtttcctttgtgggctttgaaataccaccacctatggtcttatggttgaagcatagttcagggccagtgcatattaatgggcagcacttaacagctgtggaggaagatgcacagtgagaagatgaagaggaggaggatgcaaagctcttaagtatatctggaaagcgagctgcctctagaggtggtagtgaggttccacagaaaaaaagtaaaaacttgctgctaatgaagataatgatgatgacaatgaagatgacgatgacaagggtgaggaagatgaagaaaaagctcagtgaagaaatctatataagatactccagccaaaattgcacaaaaatcaaaccagactgaaaaagactcaaaaccatcaacatcaagacccaaaaggtcaagaatccttcaaaaaacagaaaaaaaaccctcccaaaacgctaaaaggacctagttctgtagaaaacattaaagcaaaaatgcaagcaagtacacaaaaaggtggttctcttcccaaattggaaaccaatttcatcaattatgtgaagaactgcttcctaatgactgaccaagaggctattcaagatctctggcagtggagcaagaagtctccttaatagtttaaacagcttgttagaaaatttctgtcttatttcatttctctatcatttgatatccacctgtctttttgtaatgcagagtgagaaatttacataccatatctgataaatgttgtccaggttccattgccaagaatgtgttgtccaaaatgtctgttcagttttctaagatggaactccaccctttgcttggttttaagtatgtatggaatgttatgataggatatagtatagtagtggtcagacacggaaatggtggggatacaaaaatgtgtgtgtgaaataaactcattattaaaatgttttttgaagtaattttatatttatagaaagtttcaaacattgtacaaaattcccatgtactcttcacccagtttcccttaacgataactggttacataaaaccagtgtattcttaaacttttattttctacagttatgacagttatatagatatataattaatttttactaaaccttttaagtgagttttaaattttcactgatttttaattaactacccactttgttaaatttacatattaattcataatttaaatgaacatcttttaatagattttcttcatactcaattacaaatcatctgtaaatagtgaaagatttatttatttttctccaatactgatatctttttcttttttttacaatatcgcactgtctaggattccctgaacaatgctgaaatgaagcagtaagagtatcttcgtcttgttttcgatttaaaagaataatttctatatttcccctttaaacctgatgtatactgtggaatttattttgtagctatcctttatcaagttaataaacattttttatttctatttgtaaaaattgttcattatgattgggtagtttatcagctgcttttaaaacatttattgaaatatatttcttcctcattgtcttaatgtgatgaattataatgaattagtttggaaagttgatatgttatatttctagagtaaacccaatttttatgaacatttaagatcttgatgaactgctaatattttatttagggttttaactactatgactatgagaaatattctcttataatattcctttcttgtgatattctgttaagtttttggtattggtatcacataggcctcaaaaaattatttgtgatggattcttcctaccatccattcctttattctaaaacaatttgtgattggtgttattttttccttaaattcctggtagaatttcctggtgaagctatatgggttttgtgttttctcctggtaaatttagttttaaaaacccagttttgtaagtaattaaagaaccactcacatttgctgtatttttattcagtcttgcaaggttgcatttttattataatttttccatttaatatattccatttcataagtttttgaaatgtttacgtaaagtttattttaatattttcttgtcatccttttaataacattaaaatctgtagtgaagtctccttttctttcccagtgctgggcatgcatgctttttctttgttggtcagtcttactagagttcgatcaattttactacttatttcaaagaatcaacctctgactttaaaaatcttacatatatgcttgttttctattttatcattttatttatatcttctttggtttctttgcatttaatttgctgctttgattctctaatgagatatatgcttttaaacattaattttaagttttcttcttttctaatatatacatttaaaagcatatgtttctctaagttcagctttatatatcacacaagttttcacatgttatattttcataatgctttatttcagaatactttctagtatttcactgtgatttctttggacacatgaattagagtatatttgaaatctcaaaatatatacgattttctaattacctcactgtggtaagaaattatactatgtatgaattcaattagttcaaatttattgaaacttgctttatgctccatatacggtctatttttaaaaatgttaagaatgtccttgaaaagaatgatattctgtcatgttaggggatatatactccattatatatttaaattatagcaagtttatcaagtgtttaaatcttccacatcacccttcacttttattttcttcatggtttatcagttactgagagacgtgtactaaaatttgttatgatgattgtggttttgccaattttttcatttggttctttccatgtatacttaatatgttttcatgctgtcttagacacaaaaagtttgaattgttttatcttcttggttcatagaatattttatcagtatgaaacacctcttttgatctctattttctcttttttcttgccttaaagtccatttttatctgatattaatatggtcacatgaattttccttcagttagttcttgtggataaattttttttcatttccttcattcaaagtttccttataattatattacagaaagcttcccttcaaagagcatctgttattacaatgttatcacctttgtcttaattggtgaactaaatccatttatatccacttatatgtgatttgtacaaccacccaagctgattctaaaattttatataaaaatgcagtggggccaaatgtagccaagactgtcttgaagaagaaaaacaaacagaaagatttgttctagcagatatcaagacgttttataaaactacagtagttaagacagtctgataatatcacataaaaagagagaaacctgtgaaatagaatagactctccatgtatacatggataaagttaacactactgagccataatgaatggatggtcttttttttttttttttttttttttttttttttttttttgagacggagtcttgtactatcacccaggctggagtgcagtggtgctatctcggctcaatgcaacctctgcctcctgggttcaaccgattctccagcctcagcctcctgagtagctgggaatacaggtgcgcaccaccacgcccagctaatttttgtatttttagtagagatggggtttcaccatgttggttaggatggtctcaaacttctgacctcgtgattcgcccaccttggcctcccaaagtgctgggattacaggcatgagccactgtgcctggctgaatggatggtcttttctataagtggtacttgatcaactgtatattcacattgaaacaaacaaacctgacctctatattatgccatatacatattcaatgccagatgaactatgcatctaaatgttgaagtcaaaaaataaacctttcagaaaagaggaaggaaagaataaaaataatcttttcttgactttgggaatccagaaagaatacttaaacagattacaaaaagaaccaatcctacaggaaaagattgataaattataccacattaaaaagttactttcaattatcaaaggtcacctttaagacggtgaaaagacaagatatttcaacacatgaaactaacactagaacctataaagaattgctaaatcagtaaggaaaaagacaatatgaaggtggaccaaagttttgaatagatacttcaaaaacagtatgtacaaatgatcaataaatagatgaagaactgctttaccgcattagcgtctgggcaaatcaaaaccatggtgatatactactacacctccaacagaatggctaagattttaaaagactgacaatatggagtattagaaggatacagaactgtgtaaacacttgtatgccactggtaggagtacaaattgtaactgccactatggaaaacacatttaaacacatgcatatttcaggattcagcaataccacttgtagaaatatattcaacaaaaatgtcggtacaagtgcaccaagaaacacatataaaatgtccatcacaacattagatacaatagcccccaaactggaaaaattccctttatctgtcaaaagcagaatatgtaaataaactgtgacatttccctaaaagttatttttatagaatggaatactatatgtgaatggaaatgaatcaaatatagctagctgcaataacatggataaatctcttaagcaaaatgagtaagaattatatattgtatgatttcatggatataaagctacaaattaaaatatagtcttagacatcagaaaattggttactttcagaaaagagaaaggggatactagttagaaaaggaatgtgggggcttttcatagagatagagctgacattactctatctcttggttgatcattacatatgtgatctctgtgatatttcattgagttgtacatttgtgttttgcagttttctgtataattgttatattttacaattaaaaacagtgtagcaaaaattaaaaatccaaaaaaaattaaaagcactgtgatgtaagagaatagggaaacaaagtctagatcttgagttcatgttctttccactgtattactgtccttccaggaaacaaagtgagattatatattttgcataagagcaacatgttatattcttaggtacaaggccacaaatttacttacaaagaaagctgaaagtccccctgcaacatgcaaagcgactcagacaaaatgcagaaagggttcagttgtcagagtcaaagatattgtggaacgttggaagaatatgataatcaagaccaagaggacaagggaagcagattgtaatgaagacctgactctccatgctaatgaactattatcaattccctagtgaggagggattggtggtggaaactcaactctcatttgaataattgtcttagagaagtctgcaattagttgtgtatgtttaatttgattgtgtaagtaaatctggttataattttatccaaattgtgattcatgagtcattcttgaaataacctattttatttgtttgaagtcattaaaattcttgaccaaatagattgggggaaatatcagaatcagggtgattgtattgacaatcaagttactacactgacaaactattgaaattattcagattgcgtctgcctgcatcctacctacatgtggatgacatagggtgatatcaacacagcaataaagaaatatctctggtcaatagcagtgacataagtcttatgagagtaacaggaagaccaagaggtaaagggaacagtcatggtcatcagctcacatgagatattgcaggaattccttgaataaggtaagagggaccagtgagtcagactccgagattattactgtagttattgttctattaactgagttttgtccaaaactactatggcttagagaaatgtaggttaaacaatacatcagccaatgaatgaaaggaggaagactatgagaagatgcctctgttccatatcaaacccaaatagtatcccataatttccatgatgcttaaccatggacagtttgcacatgatatatttaaacatctatggcagtcaccaactaagtaattaaaaataaacctaaaaataaatggaagaaggcatagcggtcaattggaaagaggataataaatacaatttgttggatgattcagattattgatattcttgtttaatcattaaggtaagtttgtagatatacaacataatgagaaaacagttagaaccacacagtagagttaggaaaatactacatatgttgaaaagtgcatgtcaaactaaagttctaacaacattgaagggaagttcgttcagggctgtatgtgtagttttccaacataatgtctgtagtctggatatcaattgttatgaagaaggcttggctcacattaaatgccaaaaatcttgtttacttgcccctgtttctgcaagtaagtttaaatttgacagtcaaatttgttaagtcctgtttcactttgtgtgccttagtattctctttatttgcttttttaatctcttcttcctttaatctgctaattttatatttttcaatattttctttaattttttttattttattaaagagattttgtatgatactcttttccaagtgttgacttttctgctaacaagacctgtttcacattatgtagcagtggtgtgccggtaaatgttgaactgctaactgtccaagtagtagtcctaatttgtagtatttgctgatttctgtggtgtaaatattcccaccatggcctatttcaagctactaacgtgatgttattaaacttagatttaagcagagatacacacaattggctttcacaagtcatttacagtcagttccagcatattgtggttagagtataattttgtttattgattatctgggaaaattgctaatagttattttcatagtaagctttgttttcttctacaagcttctgccatctccaaaacaaacaaacaaacaaaatgctgttttgttgctgttgttgttttacaccaaagcttgggattcatttattcaaagcttccatattttttcacagaaattctaattccttatgatattccattacccagattcagccccagtatatacttttattctactcaacatacctctcctctaccaaaaacaaaacgaacaaatccaaacagagccatccacaattccaagaagtcttaatgtctttatctatgctttcccattacatgataatcttttccttagacattagctcatattagtctgatttttttattcaagaccaattaaatgttaaaatattcctcgatctctgtagttaaaaacaatggttttatgttatattcccaaagcactttgttttacttttacttatattgaaacttttgggcacaaaatacatgcccaataaaagactaatgaatggtcagataaatgaatgtcatttcatgtttgtttcaccatggcaaagaactgctttctttcttttattggttaccacaacctgtgaaatatccaggtccctggcccatattctccttaacaccctctgaaaaacctaaagactatatagaaaatttatataatggagttttgaattatccaaacaacgtccacttcatttggcatttgataaaagatataaacaaaaataaatccacaaccaatttggctctgccttgttgaatataagaaatgcaacatcacaggccacatctacactgaaaagtatcttcaaataatattttccccaaacagttttattctctttagattacccacacattctctttcagagtatggagagccttcaaagtttaaaagaaaaccatagaaaaacactattctgacatatcatacattgtccctgtattcagttcctccttcctttactcatgaatggcaactatttaggagcttgtaagctttcccagattcacaccaattccccaatggactaagcttcagccatacataatctaaaggatgaaaaagatggcaaaacaacttccctattcggaatccaatgaaaacaaacccctgatgagcaacttggacaacaacatctcaagagttcacctcaggggtggttgtcaacacttactagagtcaattcaagagactgtttcagcagctttagagcaaaacgtcatctttaaaagattatcaaccaacacaatcactaagtttagccactgtttgtgaaattcaagtcaaatagaagcagctgagcatactcaacaggagatagcagttattaaaaagaaactggagagagggagggagaaagggagagagggttgaagggagtgaggaagtgaagaataacgggagggaggcagggagggaaagaaaagaaaaaaaaaagaagagacaaaaaaagaaatcagaatccaaattttaaaaagagtgagggaaatcaaaacatgctatatgctccaagtgtaaatgctgaacttgcagtactagaggatttaccataatctcaacagcttccagacctatgacaatttgttttgcttttttcctctaacaagcttagaaatcatcatttattcggtattgacaggtgatcttctaatggccatgttctcaactcactacccaaggcttctactactacatgtcctctcctttctttgacttgcttgaagtttttcaatgtttttgttttagattccataggaatggcaactgcttaaagcttttctcacctcttgggaaaatttctttcagaggaagtaacccggggtgccactaattggctttgcataacagtaggcaaatttggccttaaagcaactgtgacactgtttgccaatcagctcaaatacagattaccaaaatcagattttctgggaacctcacagatgtttctattatgaattctaaaaactatgtggttattgacacaagtatccctaatgcctttccaagcaacttaatgattcttctcatttaaatattcacttcaatttcttgcaaaccccaagctggtgtttctaagtgcttcattgaaaaagatcaaaaatattcatcatgcactgattggactgataaatctaagttctaaactttgccaagaaaaagaaaaaaatggacttctggtatgtcttcaatatcagttgcctccaggaatttcctgtaccatgctcaaattagttttaatccaatagtatactattttgactgaccttgtacatatttctgaaaagaatagttaagagatgttttgctaagtagatcattttcatgataaagatatttaaaaagaagaaaatgcatgttttgggtgcaacactcagataactaaaatttttaaaaatcaagttttttggtacccctctatgtgtctacacagatgcacagctaacggttcctgctgattttataattactttgggttatgtaatacatattattctgctgatcataagccctaagaagcaaaattttgctgtattgacatggcctggccaaaaacaacaagggaaagaaatagcattcaaaaaactggcaaaatgtttatgaattctaatcctctgcacatgaataaatacatctgacaaagagcagaggccctcagaaaggcccctcttgacctttctaatgggagttcaattgcaagttcagtgttgagaaactaaagcgggttctgccaatgttttaagttcaagctttactacattttgttatttgtaaaagtgaaatcccagataatgagttctaattgctctattaatcactgaataaagggtttggagggaaactgggatttgaaatcgcaatctgaaagacatccttttgtatttttttacctacagcttaccatataaaactactgtgaaaactacatatatgcactttttaaaaaatggtgaagtacatgtaatacgccttatatattacttttctaaaagaataaaatttaaaacttcagtgtagtcataatgatctttgacctggaaaaaaatacagaaaaccaggccttggattataaattatattgtcaatgaaagtgagaagaactctccattctttaatgtgttttatgtattatattttcatttcctttttccaaatgaattataatgtacaacacatttgtatttttctttaatttttcacttttaataatctttctttttcttaactttgttcttccatatactgatagacctgacacaacaaaatttacctttcaaaaattcaataatcccatattcattgttacccttaaagtatctgctaggaattctatattcttatttatgttcccaagaaagtttaatgcaaaaaatatagaaaagcatacattatttaaacctcccttcctttagtttatattgaaaaaattttaggttgtgcttatgcaactgaagaccaccaaccaaaagggacaagctggtatttagggcatcataataactaactcaaatttaccataagacatacatattaattagtaagttcaatagtcaatatagccaaaaataatcatttcagttagcacttactggaaattttagcaatctaaacactcacatggtcaggtttaatattgtccagcaatacctttattcctttctctttctcaagcctggtctttctaaacatatagagaaaaggcacaggtctcacactttaaatcaggtgacatcattgtcattctttctgcgttctggtctccaattaaattccctttccttcttaggcccaaggctttgactatcttttgcaatatgcagtagattattaatgttcaaactctgaccagttggtgattttcaaacagatgcctccaaggtagccaacagttgcagtattcactctcctttttctatctttttggaagtcattcataaatttaaaagggtggttttaatatttaattcagcattttgagatttttttttattattattttttttgagacggagtctcgcctctgtcgcccaggctggaatgcagtggtgccatctcggctcactgcaagctccacctctcgggttcatgccattctcctgactcaggctccggagtagctgggactacaggcgcctgccaacacgccccgctaattttttgtatttttagtagagacagggtttcacagtgttagccaggatggtcttgatctcatgacctcgtgatccacccgcctcggcctcccaaagtgctgcgattacaggcgtgagacaccgcgcccggccagcgagatactttcatataggaatatttaagaatatgtaatctctcatattgccatcaattttttttttttttttttttttttgagacggagtctcgctctgtcgcccaggctggagtgcagtggcgggatctcggctcatgccatcaattttaaagtcactaattgctctacaaaagcagtgtatttcatctccacgaaaagcacgtgtactaaaatggccagagttctcccagtcaaaaggtcatagaatggcagcaaggtacaaaacacactttgctttacagtaaacacagataaattaagaaaaacatgtaactccacacagttgaatctgttctgaaacataatcatttcttaaagaaagagatcataggggagatcactccatcctcattggaaatgttgggttaagagcaaaagattatgagtatagagacatttgaatgcatgtgttcaaagaaagccagtaaaatccctgatttccttccacataggaagaaaagtagttggcttttgcaatcaggtaacatttctttctggctaggtcaattatccatggagctacagatccacaacctattctgattgtctgcacatctggtgtaagcctttataatgcaaatattaatattattatgcttctgtaacatatttctataattaaaatcaaataagtgatttcagaatacaggtgactatgcaaaaaatgttatctaggggacaaagaagcaccccaacatcaacttataaaataaaaatagcatttatttcttgcactcccttactgccacaaaaaaacactcaacatcctgtttgacattattggtgtacagggatcacagaaagacactaacattttagaaaattttacacacgttgaattgtgcgtgatctgaaacagcagcactttgttgacactaatcattagataattacatcctttgagttactgtgctgtctaaaattaacaagacagccaggcacggtggttcaggcctgtaattccagcactttgggaggccgaggcgggcagatcacgaggtcaggagttcgagaccagcctagccaacatggtgaaaccccgtctctactaaaaatacaaaaattagctgggcgtgatggtgcacacctgtaatcccagctacttgggtggctgaggcaggggaatcacttcaacccgggtggcggaggttgcagtgagccgagatcgcgccactgcactccagcctgggcgacagagcgagactctatctcagaaaaaaaaaaaaaaaaaaaaaaaaattaacaagagcaaagtacttgagcaatgcttaagttttctctctcatattttttcttcaaattaacaacatacatttttacttcaatatatatgaaaaataatcatatggaaacattacaggtttgtaaaataatgatgacaatagtaactatgtgttctatgtgcagaagaaaaggtacattttgttttataaaatactacaggcaaaggcatgcattacagttaaaaaaatgatatgaatgatagatttttaaaaagattttgtatatgtttatctaataagcaaaatcatattgcaaattcataaaagaaaggcaaaatgcatatgatagtcttaacactactgtataatcactatagaaaatagattaatggataatattaatgaatacatagaaactttgaaatatttgctgaattgcaactaattggttgaaaatgttgccatgagctggaagtgaacctcatagcaattggatctgaattctgcagagtagtcagaaagtatgttgcaatgtagccatgtgatgtgaaagaaaatataatggtctttggccatttagattcatgttcatgtcttggatcttataatgcattctgactgcatgactttggatggatttctcaacatttttgagactcattctcaccatctgtaagatggaaatacctacctcttagcataaagaattcttaaatggtgacaaaaatagtttaccaatttttaaaacagtttttttttcccagaaataacataaccctacaagaaacctccagacattttttaaagttttatttcttattcaatgtttccgtactcattggttgggtcagtagacaaaatgttcttatttttgagctccatggctaacctgattgaagggagtgagtgctatgtggattctgtacctgggataacattgtagtatacgagaacatgtaaacttatctaaggcttaccttctttcttaatggtaagagcaatgttaagctttctgagtatgcagaatttttttttagtttaaaaatatgcatgcatacatccctgattctttacccctgcccctttgtacttttacttttttacctcatttcctagaagggcatttacacaggcttctgatgcatcacagatggctgtgagatcatgtcctccttctagagccaacaccacacgtccatcagccaatgtcatcaattgcttcgtcaaatgaccaaaacctaccataatacaaacagaacatttcaaagtattaaatcaagagaaagtgatcactagaatctaacattgagcatgtattttgagtaagtcattactaagcattttctgtgtgctaactcatccaatacttaaaataactttatggattaggtcctattattatctctattttacagataagggacccaaagcccaagatcccacagctaagaagtatcacaaccaagactggcaccaatctagctcttcaccagtgcacagggctgcttttcataacatggtctgtatgtatctggataaaaaaactgaatactcttcagcaggtgttaacaaggtaaactcttccatttgcttaccatatgcttcctgtcgctatcaatggcctttattatagaaagttttctatgccccaaattgctatatattacaaatttacattatctaatataatatttacattatctaatgtaatgtaaaatctattacattagattttataagcattattttattatgaaacatttgataaatgcaaaatatgaataacatctatacgagttataaaatacaataatatgacaaatatctgcttttttaccttccaacttaaggaaaattaagttagcaacactttttcaacccccatgtgcctttctctgacagtagctatcctcctccttgtgggagatcaggatcctgagtttataattccattgctttccttttctaaatttccttgtttttcaactttataaaaatggttttacacttttgcaacttgctattttcatgtattttgttagctcatctttgttgatccctgtagctatagtttttttgctgtctagtaggccattatatgtcaacagaggttacccattcttctattggcagagagttggttttcttgttatcaccaaattccagtttttttgtcgttacaaataacgtcgccatgaacattcttacacgggtcttctggtgcacatatgcacatgtgaaacagaattgttgagtcttaaaaatctgctaaataatgccagattatttccaaagggctattaccaagccccaccctcagaaacagtatatgtaagaattccagttaagtcacatccttgccaacatttggtatggtttgccaagtgggtggcaaaaaaatggcatcttattgtgttttcattcagtgtaagaggggagttttctatttctaatttaacttagagatttcattacaaatgtaatttaatctaattatttctttacacagatagacttttccccataatttcacagtgctgttagttacattaattaatatttcaaatatgaagccatcattgcatttaggaagtaaaataagcttggtcataatgtattatctttttagtacacacctggatttgttttgctaatgctttaagatttttttgagtatgttcatgaatggggttaatctgtacttttccattcctgtattggccataatgaattttgatgtggttgggaccctctgtttttattatctgatgccagaatgttggggacaaatctcgtatgaaaccattagggtcttgtgttcactttgaagaatatttttagtattgattgaatttttaaagggattatatcactacacctgctttctatatcgtcctaagttatatttttacaggcgtttccttcttttatttcattgtttaaatttgctgaaataaaatgtttgtttactctattacattattttgaagttccacatagatgtaggtggacattgtctgtaaatatttccttgatctaagttctctttcatattttccatctctctctgtcctgcattacgtatatttttgaatcagagctgtcttccagactttccctcttcagacatctctaatctgctacatcacatctttagtgatattttcatttctgtgattgtattatttaactctagaattccaattttgttctatttcgtgacttattggtcattatgatactttcttaactccttgtttatgtttttatttcttcttttatttctttcagcataattattttatattctgtaactgacatttgaaatcttttggtttagtttgctctctgttgtttctgctgattcatgttcatgctgccttaattatcccctttttaaaaacttttattaatttatttaatttgtggagtactttgagcttgtattcattgtaactttatatgtgaaaaatatttgaagcttggtgcaatggactgaatgtgactacaccccaaaatttatatatggaaattctaacctccaatgtgatgtttttgagaggcagcctttgataattatgtagagtcttcatgaatgagatcagtacccttacaaaagggaccccagagagagtcctctagctctcttgttgccttctaaggatccaaggagaagttggcactctttaatccagattaggtagagtcctcgtaaattagatttgtacccttaacaaaaaggacccagagtcctctagttctcttgttgccttctaagggtccaagtctttacttggcagtctttattccagaaggccctgaccagaacctgaccatgctggcaagctgatctcagacttacagtaaccagaactgtgagaaataaatttctgttgtttataagctatcagtctatcgtattttgatacagcagcctgaactaagacactggggttgaatatggtatctgtcattcaagtctggctcaagtgtgatattgtttgcttttgctggatatctgggacactaccttctaggacctattttaaattcttatatggcaagactggttgtggagtttgctgtttttgtttgcacatgtatattataaatatggaccttaaaactttattacaaattctcatggagaagaaagtcaggacatcctttctttctttctttctttctttctttctttttttccttccttccttccttccttctttctttctatctttctttcttctttttctctttctctcttacgttctttctctctctttctctgaaactttctttcggaaattccctttattggtggcaaaagctaagatagatatgatgttttatttaggagtattttcttttctcctatttattctcttccaaagtgttggccatttgtcattctttctttattctggtctccagttaaattccctccccttcttaggcccaagcctctgaccctcttttgcaatatgcagtagattattaatgttccaactccgaccgattggagattttggtatagatgcccccagggtagtcaacaacttcaggatttactctcctttttctatcttttggaagtcaaccacaaatttaaaaggatgattttaatatttaattcagcattttgagatactttcacataaaagtgtttaagaatatgtaatgtctcatattgccaaatttgaatgtttctcattcaacatttagaaaaaattatcatggcatccgacctgtccaatttcaggggcaattgccaaaaggtacggtaaataaaaataaatgaaaacacgtgtttcagtagaacatttcattttttatatgctttgtgaacttacagcaaaaaattttaatttttaaatcaattgaatttttcataattcatagattttattttttagagcagttttgtatttacagaaaaactgagcaggaagtacagcaagttccatatccagtctctttctttccatattaacattttgcatcattttggtacatttgttatgattgataagccagtaccgatgcattattattaactaaagtatagttgaggttcacactttgtgttgtacattctgtgggttttgacaaatgcataatgtcacgcatccaccattatactatcagacagaatcaatgactgccctgaaaatttcctgtgatttacctattcatcctccctctctgcccctcaactcctggcaaccaatgatcttttaattgtcatagtctttagtttgaaaagaaaataaattctcaggatgtattcactagaatttaatgaaagcctataaaatcatcagataatgagagcctgaaactaaattaggttgcttctattttgaacaataaaataatacattaatcccggtaatgcattaaaataatccacttggtaaacaactgcctagattttcccttctggtctagcacttgatgttcaccatgaacaggatgagtgaatctcctcaatatcttgaagcactttaatgttgattttaaccacttaactactatttggatggaagtcaaattagtcactttacaattatcacgttttaaaattcttgacaaaaatacatttaaaaaggatttaaaaattagttaaaaactgttatcaagcattttagtttttctcaattcctatggacctcattatgatgccgataagaatcttttacccaggccacttaggaatgaggctgtccacactaaactaccttattaccgggatgacaataaaaagaatgataaacattcagaaaagaagtaacgcaaaattttgatccctaatgcttagaaatgttctttccctgagccaagattaatagcacatagtaattagtaccattctagaaaatttatgtaaaaaacctaataggtaaggacacatttctgttaatcatattattaaatattctagaccgctttttctgtaacctaaaccagggctacagttataattttaaaagatataataaatcaagccagaaatcttactaataatttctagtaactgagaattcaattatctaaagtcactactgaacaaaccaaatcactggattaaagaaagaaagagacagagcgatccaaatctggtttaagttcactctttcctctggcaatgaaaaaaagtcccataaaatgccaaatttagatgggatagtaaaaagtggaaggaaagagtggaatgaagttaatctttgatctctatccaaattcctctttcttttacctacagggtttatttccattcacatgttacccatgtgatctctgcagtcagagactgaggcagctgaactctaagttatgcatgcacaaaacattcaagtgatgtaaggaaagttctacaaggcccatgccctttcttcaaatcaaaataagcaattcagtatttttttttaattttattatcctgaattctgctgtgtccactgttactgtacatattagaaacattaaaaatgccagccgagtgtggctacccaggtgaaggtattatttttcaggacatattaaaggtttcagtggcatgtgtacgtgtgtgtgtgtgtgtttgtgtgtatgtgtgtgttagattagagcatatataacaattttagtaggcatgattgcaagtttcatgtaatcatatttacttctagcttcattaaagacacaaaagctcatcctacatttgacagtaaacttaataacaccttcagagttgaaaaattttagcacagaccttcctcaagcggggttgagtgttttgagtgtgagggctgatgggctaagtgattaagtggaaaacgtgttcctataccatggtaccttgtaggctcaagcaatcactggacatagagatggaacggggttctcaacaaccacatagggcatcagtttcctaattgagtgcagagctgagggaggagcttggatgcctcggagcagccacatgtacaggttcatattgggatgcactgactaaaaatcagccttgacagacatccctattacctgtattgactacaaaagaatctgtatactttataggtgttggtttctgctaagaaatcatattgtttcagatatttttttttttgaggcggcgtcttgctgtgtcacctgggctagagtgcagtggcgtgatctcagctcactgcaacctccacctcccgggttcaagcaattctcctgcctcagcctcccgagtagctggactacaggcgtgcaccaccacacccagctaatttttgtatttttagtagagacgcggtttcaccatgttggttggccaggatagtctcgatctcttgacctcgtgatccaccagcctttgcctcccaaagtgctggaattacaggcgtaagctaccatgcccagccgtgtcagatactttttaaaagagatttggtaacttaagcttttattttaggaaatattttagaatgaggcattaatcaaacaccacatctactactaaagaatcctacatgtagcctggttgttttaaaaattgtctatcagcattaaattataagcatgagaaggattctacattgtaacaaatcatttcttttgatgaccaagtggagctgaactggtaattaccatgaccttgaaaccactgagaaagagaattagaagggccttttcagaatgaaatcctctgtttggttccattaatttagtagaaacaaatttaatatttttgattataaaaatagaattaaataagctatcaatatggtaatggtttcaaattatcaatttcaatttgatttcctgatactttataggggttggtttctgctaagaaatcatattgtgtcagtattttctctagtttagaagttgtttgaaatgaaaagttgctctaagaaggctcaaagattaagccttatatacgtatttaataaccaagtcagatgacacaaaaggattcatccttcaaggtgacatgtctcaaatgcttctctctaacatttccaaatagttcccagagaatagtggtagtgaaaggaaacaaccttaactagctttattttaattttcattaaaaaaaactatattaaaaaaccaaaattattgcattctcgttgtaagaaatttggaagatgatagaaaataataaataaatagtataataataaacaggctctatgttgtgatatatcatataaagtgttatcttttatttgataaataaataaaataaatgatcatcatcaaataattataaataataataaatacatagttccatcgaccagaaatagccaattctacctttgtgtatttcatgcttcttgttgtattttctaaataaaacaaaatcattctggttttttaaaaatacatttacaaatatcaaatagtggatacatttttcatgttctctacaatatcatttaaaatgtacccatgcaccaagcaattctgtttttagatatttaagtgtgctttaggtttttagtttaatatacaagatagcaaataatagcctagtgtataatcaaagtgaccaatgtatgttagtgtttatcttattctagtgttattatgtattattagggttggaagaagccttgctctttttattgctaacttatctgtttcaccagagcatgggctagaacctaaagcacataaagccaaaaggagaacaaagtacagtcagaactgtataaacttttttcttttagaacccatgtatatttagcaatgcctattttgaataacctaatgttttgatagaagctcagagaaatgagagattctcccacaaaatctgtttctattacaaaattgcaaatggaattggaagtctctatggaggccaaaactgagttaatgctgtagtaggttgaacagggaattctctccaacctacaatttagttactattgctatcctgcctccctgtagtaaaatagaacagactctagaatcagcagccaattctcagagaaagatacctcgatcatgattcgtttggttaataaagaaatggtgacacattgtgatctattggataagtcattttacttctacttaaacatttgttgacgttgctgatgccagtctcccattcatgacaagtctccccccaactattattttctttctattgaggaaagcctctagttaaaaaaagaaacaacaaaaatgattctggcaacctccatcctcactctgccattcagcagcagaggcactggcatcaagataagcaggagtgaaagcttttgaaatactcactgacacctatctcatgatgatttgtattaatttgtaactcttctatggctaaaaagtctcactaccaatttactcatttattaacatgtcaattacatttgggcctgaaatgtttatgaagacttcaccacacatttaatatagtagggaccaaaggagtcacacattgttgccctattttcatttgaaattaaatttttctcatccattggactaccttctgtagttatatccaaaaagtattactgaagaatgcacatccagggaagtctaattataaaaactgtagccattttcctccctctctaaaccccctggtcattaagttaccatgttattctttaggaaaatgcagtcaggctcaagtagagagagaaattcgggtagcatgtggcagtctcatgtattgtgaggctctccagcaggacttcaaagcagaatctgattgttgcagatgggaggaatgctttccacagaaatacctttttaagactcatggtgctgtaattcatatggtaggcacttgtgattcattcctgatgatgttgggatcatttaactcacagaaccattgtcgctaccataaagtcctttcatctgtagccaaaaaggtttatttcatgcaaatgaaaaattttcattaaagtatcaccttaataacgggtaaagatatatattatctggttatgtttaaaatgtaaatttatgaacatattctgaagattcatttcatgttaagattttacttatttgatgtcccaggactctttccaagttcactgaatccggaaatcaattttataagaaatatggagattattaccattaaatctttcaattggcttattccaagcagccctataaatactgcatatgttttaaagaaagcattttcaatcacagtaaaaatccttttactcttctagtcagtgatgctccaggaggtaagtttatattctcagcaccttttgaaagcaaatgaataaattgtatcctaaaaaaagtctgaaacaacagaatacttcaatgccattattttttgtaagacaaaattgatcctcagaaattcctgagaaaataaatggcaagaatgattgactggaactttagttatccatcaatccatccatccatccattcacccatccctccatccatctacgatccaactacaaatcaactattatttacaaaatacctactctgtgccacagttttatacataattctgatacttctattagatatgagttattataaactctatttgaacctcagttatattacctataaataaaattaaatactacttattctacatcacaggtttgaaatgaatatttaatcattaaaggccaactgcaaaatggatacacagtttatcaaacagagtctggcatatggcagatgctcagtatttatacatttttttggagatgaagtctcactctgtcatccaggctggagtgtagtggcatcatcatagctcacttaacctaaaattacagggcccaaacaatcctcccacctcagtctactgactagcttggattacatcccactaccacaccctactaatttttcaattttttttataaggacagggtcttactatgttgcccaggctggtctagaactcctggcctgaagcaatcctcctgcctcggcctcccagagttctgggattacaggtgtgagccaccatgcttggccagtatttatacttttaatgaaagcttttcatttaacaattacagatctagatataattgcaagtttacatactccaattctatcgttttaagaagtggatgaggaaactaaggcccatagtgataccagagagcaattttttgaggaaaagtaaagaagagcaagtaaaacatgaaaaatgttatgctcttatgatatatctgctatagaatatctagtatcctttttgaacaatgtttttaaaaaagacattgtggccaatataagtagaaaatcatgttcaaagatgggggtgagggtgaggagtgagagatgtgtggggaagaagttcaagtccaaataaatataacacaccaaatgaaaaaggctcaagtctttctggccacaaactcttgcttacataggtgtatggaaaaaaaaagatgtatttaactaaaaaaatttaacttatacaaaatttcattgatttagttttacacagggtaaaactaaaacaccatgtattcaagaggactcaaaaaataattgtggtagatccattcaattaagagatacctactaagaagctactatgtgaccaaggaactgtgctggcaatgaaggcatagttatgagccaagcaaaggtgttcattacccttatatggtgtataaactaatgttagtgagacctaatataaccaggcaccatgctaagtgctgaaatgcattatctcatttattcaccacacaacttcccaagttataagaacattaacttgcccaagcaacaaaggtcaatcaatgacaaagttgggataagaggttgggtcagttgactttagtgcctgtcatccaagccactcttctgtggctaaatccaagtaatattgaagtgcaaatttaatgcattagtactacaatcacagtgccagttgtgcctgaaaaataatcctcaaacgttaatgactgtaacccattcttcttactcaagctacaacttacagtagttaaaactgatccacttcgatttttttgctattttttcagtttgaaaaggaaatatatcaccccttcaaaaaactaattccttttcaaactaacccttgcatctaagcttgcattttaactttgagcacagcattaattcatggcagtactcccaaaattcaactcaggttatgatggccatggcaacacttataattgaccattgccaaaaagcttatgcactgatttgccataatcatcctcacggtttctgaatgcctagttgctttttataaactgatattttcaactagcatagtacctgacacacaataagttatctggtctttaaaaaaacaaacaaacaacaacgaaaatattactattgaatctcaatgtgtatattcttcacaaacagatgatcattcatctttaaagtgctagataagtatcagctaaattacacagatttgttaaatggtagaaaaacaaaaccgctgccttctaaggaaaatggggacatgtctcattgccaaaaacattccttggaattgcatttcccaaatgaccagggtttttaatttcaagaccaaaatacctgattttaaaagataagtatctaccctctgggcaaaactgatgacttcttatttttcctgccataagtcgaggttcaggaaccctccgaattgtaagttacaagcaaccatttaatttagattaaattagacagcaattgtatgttaactaaatatgaaatgcctctaaatgtgtttgttaaagattaagaattccatagtatataagcttctattatacatttgttattgatgatttttaaaataaatcaccatttaatagaaatacttaaagaatatttgcaaaagaaaggataacatttagcaaaattcataagcatctaataagcccaataggatagttaggatagttttttttttttccttccttttttttaaaacaggcaattctccaacatcagggcagaaaatccgcagtacaaacatggccaagatcctacaccatttttacaaatgccatgattcaacctgtcaatatggataaaataaaggcttcttttcaaatacttatcacagtggttttgttctgttttaagtctattcccacctgccattaaaaaaatcattaaaagaaaataaagactgcctccaatttccatgaaagatttccatataactatcattctttggggaataacattacatattccatagcgtattggatcattgtttttatcttgcatgattttcctacctttccaagttggaggtgtgggacatgaaaagggagtctttcctttattatgccagaggtctttcatcttaagccatggtctacttgtgagtgaagcccaatatccaacttatataaaatgctataaaaccttcataatggtaaagatagagtatttcgggtaaggcggtgacattttaggtcaaacacttcaagacacttaaggtatctgaaagaagatgacaagattgtggaattgaatgatgagagagtgaggtaagcagaggacagattcagggtggggagatcaaagataaagaggagttgccaggactttggggaatagctgggtatgtaccagaataaataaaaaagcactatgctagccattctagaatcgttcaaactgagaggtcatggacatctttcaacaagggtctataatgagattaggcaactacttttcaaaccaaagaagctcgcagatgcattagactgggagtcaagctggaatacactagggatacgcagctgttgagtctattgctctaaccttagagtgtgagtttagatttttcaaaaatagttaaaatttcagaatctggatattaacggatagatgtataagataaaaaaagtagcactttattaaagtgggaccatcagcatttcacttatcccaatcacaagtattatagcttcagaaaataatagcaactgggtgttcaaaattacctaattaataataggtgacaaaagaaattcatagtgactattaaaggaataaagcttttatcattatcaccatgtgtcaaaagagttgtgtaactcatcctaataatttccaacttcaaattcattgaagagacattacttctcttaggagacacccaggcgttctctgccagctgcttaaaccctctctagacatttgtcgatttttattaccataaaaatgttaactgcttaggaaaattatctagtctaccttgggaagcatcagcaacagagccagggtggcaccactgattctgaatttggataaaattaatcaataatttcaaatgatattagtaactaaatctaactcaggttctatagcctactgcataattggacctgccaattcccatctctggactttgctttgtttctctaagggtcaaatataagtgtcagactaactcatatttcttaaagtggagtttaacctttggttaatcagaatctcctgggtgtttgttaaaatgcagatttgtgagcctcatcccagaccaagtttgaatccaaatctctgcatttaaagtaagttcctctactgaggtttgagtttttccactgaggtccttctacctgcattgaggtttgaaggtcatctgactacaaaatcttgaaggcttccttctagctgtgttatttgagttgatcctctcacaacattttttatttttttgggaaaaagaaattagactatcattacattataaataataggtttaattatatagaaaacatatagaatttaaaaataggataatttagcacagtgtcttgttataaaagcaatttataataattaatgttttttaattattattattatagtttaagttgtagggtacatgtgcacaacgtgcaggtttgttacatatatatacatgtgccatgttggtgtgctgcacccattaactcgtcatttagcattaggtatatctcctaatgctatccttcccccctccccccaccccacaacaggccccagtgtgtgatgttccccttcctgtgtccatgtgttctcattatgcagttccaacctatgagtgagaatatgcggtgtttggttttttgtccttgtgagagtttgctgagaatgatggtttccagcttcatccatgtccctacaaaggacatgaactcatcctttgttatggctgcatagtattccatagtgcatatgtgccacattttcttaatccagtctatcattgttggacatttgggttggttccaagtctttgctattgtgaatagtgccgcaataaacatacgtgtacatgtgtctttatagcagcatgatttatagtcctttgggtatatacccagtaatgggatggctgggtcaaatggtatttctagttctagatccctaaggaattgccacactgacttccacaatgattgaactagttaacagtcccaccaacagtgtaaaagtgttcctatttctccacatcctctccagcacctgttgtttcctgactttttaatgattgccattctaactggtgtgagatggtatctcattgcggttttgatttgcatttctctgatggccagtgatgatgagcattttttcatgtgtgttttggctgcataaatgtcttcttttgagaagtgtctgttcatatccttcacccactttttgatggggttgtttttttcttgtaaatttgttgagttcagtgtagattctggatattagccctttgtcagataagcaggttgcaaaaattttctcccattctgtaggttgcctgttcactctgatggtggtttcgtttgctgtgcagaagctctttagtctaattagatcccatttgtcaattttggcttttgttgccattgctttggtgttttagacatgaagtccttgcccatgcctatgtcctgaatggtattgcctaggttttcttctacggtttttatggttttaggtctaacatgtaagtctttaatccatcttgaattgatttttgtataaggtgtaaggaagggatccagtttcagctttctacatatggctagccagttttcccagcaccatttattaaatagggaatcctttccccattgcttgtttttgtcaggtttgtcaaagatcagatagttgtagatatgcggcattatttctgaatcctcaataaaatactggcaaaccgaatccagcaacacatcaaaaagcttatccaccatgatcaagtgggcttcatccctggtatacaaggctggttcaacatacgaaaatcaataaatgtaatccagcatataaacagaaccaaagacaaaaaccacatgattatctcaatagatgcagaaaaggcctttgacaaaattcaacaacgcttcatgctaaaaacgctcaataaattaggtattgatgggacatatctcaaaataataagagctatctatgacaaacccacagccaatatcttactgaatggacaaaaactggaagcattccctttgaaaactggcacaagacagggatgccctctctcaccactcctactcaacatagtgttggaagttctggtcagggcaatcaggcaggagaaggaaataaagggcattcaattaggaaaagaggaagtcaaattgtccctgtttgcagatgacatgattgtatatctagaaaaccccattgtctcagcccaaaatctccttaagctgataagcaacttcagcaaagtctcaggatacaaaatcaatgtgtaaaaatcacaagcattcttatacaccaacaacagacagagagccaaatcatgagtgaactcccattcacaattgcttcaaagagaataaaacacctaggaatccaacttacaagggatgtgaaggacctcttcaaggagaactacaaaccacttttcaaggaaataaaagaggatacaaacaaatggaagaacattccatgctaatgggcaggaagaatcaatcttgtgaaaatggccatactgcccaaggtaatttatagattcaatgccatccccatcaagctaccaatgactttcttcacagaattggaaaaaaccacgttaaagttcatatggaaccaaaaaagagcccgcattgccaagtcaatcctaagccaaaagaacaaagctggaggcatcatgctacctgacttcaaactatactacaaggctacagtaactaaaacagcatggtactggtaccaaaacagagatatagacccatggaacagaagagagcctttgacaacttttattactttttagtagtcaacaacttaagcataccaaaacaaaaataaaagacagtcagattttgatttgcttatcaacaagataaataatagtatacattgttattccaggtctaggacttccatgaaattgtataggaggtggaatttcatcctggtcttttgcttctcccctgcctcccacctccctttcaagtgctcttttcttcttccctttaatccctgtgaaatcttctttgaaacaatgaatttacatcaatgggttgtttttatcaggtggctgtagatttgggagacacatcccaacatttaaatactaatacttgcaaaactcatagaaaagtgcctaacattgtaagacttatgtaactgcttgttaattctagaatgatacaacattttttcttctctaggaagattgcttaagtggacttgtgtcctggctctcacaaaatatagtcctaaaggaaactatttactcacatttaacaccaaactggttaccatcatattggttagctttatgatgaagtataaacagttacatgccaacatgaagaaaaccttccaaaaatagtaaataaatagtttaggtatcacgataaggggcaactccttggaaatgataatttgcaaaatgatacataagcgcccttctgactttgtgtttctgacaattataaattttccttgaagtgcattggctattctttataattaggcctttacattaattgtggcttattactgattaactcaagtatttattgcaaaaagttttctatgttgtaattacctctttattaccaacgacttaaaagggcaaagacttgatttgcacaggggatctgaaacatgtaatattactaaacaaaagcaacttgggctctattgaaccaaccagagaactaaatatgagaggccgtgattctcaacacaaaacaattgttcaggcccctaggattaaaactaagtttttaaacaggagaactctcattaattccaccgtaatcatttaactacaaggaaaagcaggttctttggatacttcatcatttcagctagttgattcaatagaataatttcatgttttgtttctggtgaaaacaatggaggtggcacaatttgtagagctatgcggtcaaactgtgttatgaatgcattacccaagagtatattaatcttttagtggaggagagacagacaataagtgtgaaaaggtagtaagactgtggttattttggtggcttaacttaggagcaacttttaatgatcacctgaaaaaagtctaaaattcttatgtctattcttaacctgagtaataaaagctataaaaatcatatattaagtgtgctatataaaatatttctagaataatactctgttattaacagttttatctctcttgctttatgaaacactcttctcacctggcctagtgccatcacattcttggatttccccctttcccctccatcccccccatgtcaagctgccagctcctctacctccttccacctgagaaacatgggtactcctcagagactggcctcagcactccattctccctctgcaaacatggttcatcagtgaatgttttcattgtcacagttatacccattatcactgaaccaaagactgaaatctccttttcttcctgttcgccttttctagcccttatctccaatcagaaatggccagaaatgcatttctttgcacatcgacttgattttaattgaatgtgtttaaatattatccattatccgtcatcctcggtttctcctcctgatgtcactactttggccaataggtaatcaagattgtgaaaccttaatgtctgtcaacgctaatgacacactatctcaaactacccaatgacaaatgagcttaaactgctcagtttggtaatcagggccccaatatgccgttattgtcaaaatgactgcactgaataggctttccattgcatgtattagataagagcatagatttgggggcctgaatttcatctctctcacggtcatcagtgtcatctcagacatatttttaatttccctgagtctccctttcttcttcaataaatcggggatagtaacacccaaagttaaggtgaatacatgaaaaaaatgtacttatttactttgtccaatgaaaggacacagtaaaagcagcaaatggttgctggagaacaaatgtgatctcttcactcgtgtccttctttccatctgcttttcctcctcctctcatctatgtctaacttaccactcaagaggaactctgctgcccccaccacaacgttcagacaagtggctctgatcactagacattttgcttgcagaatttgctcaatcagatactgtcttttttcatatatacgtgtcttttctattttcaatttatttgtgagattttgaaagagtagttcaacatcctaattcttggcacatcaaccaataaataaacacagtagtatttactgaaaaaaaaatcccagttttttttcaaattcatatccagaggtccagagggatgcttcaattgtgcagtgcccatgcttgatcctgagttctccctcatgactgactgcaaaaaccatgacagtgtctgagcggagagatcagtgcttggaccttctccttgctctttcctaagtaacataagatgatttcatatttgcgagctttgaaagcatttcagctcatgtttactattttctgtctaccacaaatattcacagaacttctaagagcatattgaaactgaagtgtatcatctctgattgggcacaatatactctcagaatgtattctaacatcaataaaatgtggactcttccccagcaatactcaggaaagcctcttaggttccggaaataaacattctgggatgctctgcaggccagatgtgcagactgttgagatggcattgtgggagagaaaagaaagaacccaagcccattaaacttaggatattcccagagtggctttacttttctctgcttttctttgcgaagacatggccagcaagtcctttactcctgttttctatgtatgtacactttttggttgtttgtgaatattgattaaataaggaaggcagaggtgtttgggagtaatggctctgataggcgggtagaatgctgcacttaaatccaaagcatttgtgtgcccgtttctctctgaagctcaatagcttctatatctatctcattagccacataatagaattcatcaacattttacaccactttatgtgcctgtttctctctgaagcccagtagcttctacctgaagtttcaatagtttctctctgaagcccaatagcttctatctctatctcattagccacataacagaattcatcaaaattttacaccactttactaactgtattattttgagcaaaacactttgccaccctgagacacagtgtcctcactttctcatttgtaaaactaagaaattattgcagatgatatcaaagtccccttgcatttatgattctacattagtcaaagattcatctctaccaccacatcacagagctctgtgggtcaaactctaattactctcttcaatgctccaagaaatggctgccaaatttctggctagtgactgacatttcatattcacgctagtacactttcaaacacaaaccatccctgagcctttctttgttctttggagatgtctgcttccgtcatcccaagaaaatgcaagaacagccaaatgcaggtgtttgggtatggcctaacgactaaggttctttgtctttttctgttttttttttttttttccagcggggggtgagaggagggtctgttttaaagtacagaaaaagtcacaaaggctgatgcgaagccttaacatctactctcgtttcctgaaattccctctgaagtctacgggaactctgcactctaagggcccagtcccataattggtaaatgagaaaatatccaaaggaacacaattttaaagttaacaaaaaaaccataaatagagaaaaagtgattttgttgttgttgttgttgttgttagggaaaaaattggagttaaaatggcatatttatttcgttttaatttctgggctgtcagacatcagctctgaagccctgattcagaacacatgcggggtcgctttgaagttccgactgctttttttttttttaacaatggcagaaagatataaagaaattaagctgcaacataatcataaaacaaaacttctggactcctgatttgcaaactgtcattctttcatttgttcaatgcttcattttcaaagcttcattgtggcacataattttttgtaattttttcctttaagctcgaccttagctattcttttcctctccaaagtgaggcattctacttttattttattcaacttaaaaaaaaaatagtttgttgcggttttctgaaaacaagaaaggaacaggaaaatccagagcaatatagtaaatggaatctttttgataaaagccatagcaatatagggtttagctcttaaagtaaccagttctcttggcataaagacaaaaatgtaccttaaattttcaggcaatctaaagaaaatgtctacttggttttcatgctctacttataaaatatccttacagaagattttaggatccttaataaaaactgtgcatttcaaatatggcctgacagatgccacatttgaaaacacacatcccctacacatacagacacatgcacaaatgcgtacaaattgatttattgttgtcagtcagtggtcatatatctttatttgaacaagatacacaagattttgtaatccaaccatcccttttaaatcttaagcccacaaacaagctacaagtttcttagtccccaaaatcatgtttctcattctgcttttttcctataatctcttcttggttgtattatgtaaagtattccaatatttatactctttccaaatactgctataccaattctatagtgtaacttctattccagaggttttttctgctatatctttaacggtcaaaatttcaaaaaatatgtttctctcccagcgtaatgacctttaatacacttacagtgactactagtcatcttttacatcaagttccaactcaatacatcaaagttaagtgggaaattgaaatgactatcccatcaatggtgtgctaaagccagctcatactgacttgcaagaactgacagctaaatcttcagaaatttgtgagcctggtttaaacaccattataaatgttaaattatataaacttacaacttaaaaattatattagaaacaaaggcattatgtaccccaaattcattatttctgaattattttactatatttgctattatttgtgctatgggggttacttatgtctattgtacagatagagttgaaatactggactatggattgctgttgcatgtcttgtctcagctgtgctagttacttccacattgaaaacctgaaacgaatgaatagccgggtggaatgccttcgtctgtctgggctgctgtaacaaaataccataaactgggtagcttacaaacaacaaagacgtatttctcatatttctggagggtgggaagttcaagatcaagcagaagaaaatcagattgataggagattcagtactgggtgaaggcccactttctggtgcatagacagcaccttgtcaccacgtcctcacatggtgtgagaggcaagacagctctcgatggcctcttaattgagggcactaatcccatttatttagggctctgccaccctcatgacctaattagctctcaaaagccctacctcctaacattataacattggtgataaggttttaacatatcaattttgagaggacacaaacattcagaccatagtagtgtggatattttccttggttttgtttttttgtttttgttttttaagagctactttttaaatatttacctgcaaattactaattgtcttctgtaccagaattattgaaagagctaatttacaatttacaatgagtacttatattgaatcgtggatgcttcatccttaacttccaactgttttttaatccattcactgctagtcaatatattatctttttaaattcttatcttctagggttttaacttctcttctgcctagaatgaaactgcattttcacatctactgaaccacctctcaaagctttaatgaaaacttacatatgctgtgatgtcaatattaattaggaaggctgctataagcgtcctcttattttctggcacagggtcaggcacatggagtccaaaggaaacagaaagaaaattacgcatattatttgtttaaaggtgggtggaacaaattgtgtattgctatgtcattaccttagaattttggactgtaaatgtctagaagggaggtaccacgtatgtcaagtagcctgattccttaagtattaaataatcatcagatagattgccaaacatcctcccattctattttgacaacagcctacatctataaatgaaaaacatttgtgcatcaagtagtttaaattctattattctgacttgagtgactgtcttgaatacaaacagattttagacatggcgaattaagatatttttatccagaagggatgtggaagatatttggataagaactggagaaagttcaaccgtggaaattagccacgtgttgaaaagtttgcattatttatatatacttatacagcctttgttctagtactcaaaaatgctagagtcctctctgacacaaaagagaaatagaaagtttttaagcagagaaaatatccatttgtatgtgctacaactaaatagcaacgttttcactgaaaactctttagttttctaatagttaagatcagtattatttacagtgcagtgaattactaaacacattatttaagctctagcagaacatacatttcaaggtgcatgagcatattcttggtgaaattataatacctacctctcaaaccttctgtaagtttaacacaaatctgtcttacccaaacttctgagtaataggggattactctaattttttgttagaatccttgcgatttatgcaatttacagttgtctcatttttgttttttaaaattaaaatctgaccaatatttcttaatgtccatactattgagatttcagagcagacgatgctttgatatagagaaccgtccagtgcactgtagaatgttcagcaatatccttgggcactacccactggatgctagtgggaccttcaagttgtggcaaccacaatgtctctagatactgccaaataatccccaggaaagcaaaatcacctccatttgagagcaactaaattaggctaattttcagagaaagtggtaattcaatgcataacatattttcaaatgtttcttatttattgataaagaaaacaaatttaatattaatacttatacccacttattggatacttttatagttttctacagttttaaaatattatctcattttattctgataaaaacttaggggtaaaggtttttatgttcattttcacagctgagaaaagtgaacataagagaggaaataactccctaaagctcgctcatctatagtgctggtcattacatattgataataagatgaattgagcaagagtatttaacaattgtaaatacccaacactggagcacccagatacgtaaagcaaatattattctatgtaaagagagagagatatcccaatatgaaaatagttagggacttcaataccccactcttagcattgaacggattatctagatagaaaatccacacagaaactttggatttaatctacactagacctagcagacgtttaaagaatattttatctgacaactgcagaatacacattcttctcattagtacatggaacattctctggaatagatcatatagtaggctacaaaacaagcctcaaaagatttttaaaaattgaaatcatatcagatatcttttctgaccacaatggaataaaattagaaatcaataacaagaggaattttggaaactgtacaaatacatggatattaaacaacatgctcctgaataaccaatgggtcaatgaagaatttaaaaaggaaatttaaaaaattcttggaacaaatgaaaatagaaaaacaactcaccaaaacccatgggatacagcaaaaacagtactaagagggcagtttatagcaataaacaatagatttcaaataaacaacctaacaatgcatctaaaaaaaaactagaatagcaagaaaaaccaaaccccaaattagtaaaggtaaagaaataatgtttcagagaagaaatgaacaaaatagagaccaaaaaatacaaaagatgaacaaaatgaaaagtttttaaaaataaaagataaacaaaattgacaattagctagactaagaaaaggagacgagatccaaataaacaaaatcagaaatgaagaagacattacaactgacaccgactatacaactacatgacaagacattacaactgacaccaactatacaactatacaacaacaagacattacaactgacaccaactgtacaactatgcaacaactataaaacggacacaagtataaaccaacaaattagaaaacctagaggaaatagatacatttctggatacatacaacctaccatgattaaaccagaaaaaaaaaaaaaagcctgaacagaccaataacaaataacaagatagaatcagtaataaaaagtctcccaacaaagagaaaccaagaactggaggtcttcaaagctgaattctaacaaatactggaagaataaataacaccaagtcttctcaaactattccaagatattaaaggggagaaaattcttccaaactcattctatgaggccaatattaccctgataataaaaccaaataaggacaaaacaaaataagaaaactataggctcatacccccaatgaatatagatgcaaaaaaatcctcaacaaagtactagcaaaatgaatacaaaagcatattaaaaagattatacaccatgatcaagtgatcatggatgcaaggatggttcaacttagacaaaacaataatcaaggtacatcacgtcaacagaataaaagatgaaagccatacgatcatcacaatagacacagaaagagcatttgaacccgcagccaactgtgatgaatactatactgaatgctgaaagctttttctgtaagaactggaatgatacaaggatgctaactttcaccattcttattcaacctagtactagaagtccgagtcagagcaattaggcaagagaaaaacataaaaggcattcacatgggaaaggagaaagtcaaactccctgcttgcagataatataatcacatataaagacctaaagaatccatcaaagaattccttgaattgacaaattcagtaaagttacaagatacaaaatcaacctacaaaaaatcagtaatctttctgtataccaatagtgagctacctgaaaaagaaatcaagaaagcaatccaatgggcaatagcttcaaaaatatatgagaaaatatttaaccaaggaggtcaaagatttctatgatgaaaactgtaaaacactgatgaaataaattgaagaagacacagaaagtaaaaaacatcccatgtttatgaattggatgaattaatattgttaaaatagccttgttacccaaagcaatctacagattaaatgcaatccctatcaagttaatgtaaagatgtaaagagagagattgatcccaatacaacaatagttggggacttcaacaccccactctctcagcattggacagatcatctaggtagaaatatcattctctgcacaaataaaaaaaatcctaaaatttgtatgaaaccacaaaagaccccaaatagccaaagagtcctgagcaaaaaaccccacaaaaaaaacgaaagctaaaagcatcaaactacctgacctcaaaatatactacaagcctataataaccaacatatcttggcagtggcataaaaacagacacatagatcagtggaatagaagagaggacccagaataagcccacatatatagagtcaactgatttctgacaaaagtagcaagaacatacgttggggaagggacagtctccgcatcatgcaatatatccatgcaatgaacctgcacatagtatgtaatgaatctaaaatttaaaaaatagataaatagtgctgggaaaactgaatatccatatgaagaacaatgaaactagactcccatctctcaccatatacaaaattcaacttaaaatggattaattacttaaatggaacacccacaactacgaaactactaaaagaaaacataggggaaatgctttgggacattcagctgggcaagaactttatggataaggcttcaaacaccaggcaacaaaagcaaaaataaaacaatgaaataatatcatactagaaagctgcagagcaaagaaaacaatcaacaaagtgaaaagacaacttgtagaatggaagacaatgtttgtaaactattaattaattaatttccagaatatatatacaaggaacccaaacaactcatcagcaaaataaaaccaaataatctgatttaaaaatggacaattgatcagaataatcatttcttaaaagaagacatacgaatggctaacaggcatatgaaaatattctcaacctccctaatcatcaggaaaatgcaagtcaaatccacaatgagatcttgcttcaccccggttagaatggctattatgaaaaagacaaacaataactagtgctggtgaggatgtagagaaaaatgaactcttatacactgttcatgggaatataaattagtacagccagtatggagggtcctcaaaaagccaaaaatagaatgactgtatgctccagccatctcacaactgtgtgtatatccaaaggaaggaaatcaatgtgttgagggaatatctgcacttttgtatttattgcagtactattcacaatagccaagatatggaatcaacctatgtgtccattaatggatgaatggatagagaaaatgtggtataaatagacaatggaatcttatacaaccattaaaacagaatgaaagaatgaaatttttttttgtcatttgtggctctagatgagccaagagaacattatgttaagtaaaataatgcaggcacaaaaaaaataaataccgcacaatctcacttacatgtggaagccaaaagagttgatgtcatggaagtagagagtagaatagtgtttaccagaagttgggaagtgagtggagagagggagaaataggaaaaggttggttaatagatacaaaattacaggtagataggaggaataccttctagcgttttacaacactgtagagtgactacagttaacaacaatttattctgttttccaatagctagaagaagggattttgaatgttcccatcacaaagaaatgataagcgcttgaggtgatggatatgttaatttaataatcacacattgatttaatcattacacactgtagagatgtatcaaaatattactctgtaccatataaccatgtacaattatgtgtcaattaaaattttttttaaagagtacaaagaataaaaaaattaagctctgaatacataaaggcactataattgatgtggctgggagcttctatattatgcttatttctagaatattaatgtttttaaagaatatgcataatttgagacatttcatgtctcaaattaactaactattccatgtgacatttaaaaatggtccatataaagtgtttttctactatattgtaagaggcagtataataaatgatgccttttcctttagaataaaacattctatcctggaaaaatcactgtaaaaatggctaaatacatgatatatacatagtatgtgtacagaataaaatacattatgatgataccagaggtataaatttaatcagatccaattctaggggagtggctccgtttcaaacttctgcatatggataatgctacaaatgaccagacattctaagaattactctcaagatgtcactaccctaacaaaagtaaagtaaactgtaagaatggctcatgctctctcaagtttgccctttggtgagttttgttttctttccagaattatctgattgtgcattattggtggtatgatacagaggattataggtccacctttgcccaaggaattatctagctatagctccaaagccctgtggcccttcccagaaagcctgtggtccttcctttgaccttttggcaaattttgcttgtcaaataatatgacatcacaaaatctgcagggaattcttacactagcaataaggctcaactaggaataaagctaggtgatataatcttgacatgcatcatagcatcttataatgtgcccctctttaaaaagtcattgtaaacaaaacttcatcactcgtgaattaagtaatttagaattcccattctagctcatctagtgacaaatgtgtttaacagttgttctgggcattgttctatttcacttgggaaatatttttcaaatcacagaattccattgatttatgtgagactctcccttctcttctacattgagctaaattgttttccattcaaacagaattataaagaaaaaatacacctttccatgctctgcatgggacaacatcctgcaggattgactcctaccctcgagatcatttgggtttaattgcaggtggtttccatagtgcctccttgtggtttctatgcttttggatgcacttgcagctggtttcttcttttcttgtgagggtgctcccgatgtgaaaagagagatcttccttccatcttgtgttttctctttccccaagtatacttccaaactgttgtacctccagcacaggagtttctggagaatcacaggtttaaatcagggctttccagggcttaaaaaccttatgaaaagtgcagtgtactcatggccatatcagctgttatcacattccttttgtcagtggtcctcgaaattgattgtgtgtgtattgaataaagaacaggtggattgtaatctctagctgttaagacaaggaatctgcattttaaacaagactgctcaggttagttgaatctatttgtaggaggccgtcaaacaaggtcttcagagtcactgctgtatattttctggagctgtgaaggagcaaataaactgggcttcctttgccactgttgcatcctgtaatctctttaccatcattttccccaccatagatcctctacaaactctatttcatgcatttgtagcagggcagaaaactatattatcttaacaactcaaaatgtttcttggcagaggaactatctgtacttagaaaaagacatttatatctgtatttagaaaaagacatttggcaacatggctcacgtctgtaatcccagcactttgggaggccgaggcaggtggatcacctgaggtaaggagtttaagaccagcttgcccaacatggtgaaaccctgtctctactaaaaatacaaaaattagccaggcatggtggcaagtgcctgtaatcccagctacttgggaggctaaggcaggagaatcacttgagcctgggaggcgtctaggttacagtgagttgagattgtgccactacactccagcctgcctgggagatagagtgagactccatctcaagaaaaaaaataacgttaaaaaaaaaaggaaaaagacatttgaaagtgaagaattagaagcagaggttatgggtcaatgagacaaagcaaaaggagagaaaagaaaggatagaaaagagagagagaaagaaagcaaaaagaaagaaaaaataaagagagagagagaaggaaggaaggagagaaggaaggaaggaaggaatgaaggaaagaaagaggagaaagaaaagaaagatgagaaagaaaagaaagaaaagaaggaaaagaaaggaaagaaggaaaagaaaggaaagaaagaaagaaagaaactattcagcattagaaataactataaaacttgatgagggagaaggaagaaggagtgcgagtgctaagtaatatgttaagaggttgtagtttaaaatgcacagatggctgaaatacttctagaaattggaatgttacatttctgtctcatctgcaatggaaatcccttccatttccctgcatacaataaatgctttcatacacaaaaacttgcaggtttgtttcttcatgaaaacattttaaaagggtttaattttacattagcattgatattatgcaatgtaaaaatggcactgttgagctgtggcaataatctttaacataaagttattaagaggaagcaggcacacagagctacaacattcaagaaactataagtatcacactatcctcaccacctcacttgtacaatcttaagtagaaaaatggactttcaaaaatctacgtgaagaagctttgaatttagcttatctagcttctgagggacaacattgtcttaatgaacatctactctgtataaaatgccttattaaaccttcaagtgcctgcaggctggtaagagatatatggcaaggccacagcttacaacagcaaaaaaagaagtgagcaacaagagggattctgacagtgtcatgagtggttaacaagaaatggggaggctgggccgggcgcagtggctcacgcctgtaatcccagcactttgggaggccaaggcgggtggatcacttgaggcgaagagtttgagaccaacctggccaacatggtcaggttgctaaaaaaccaaaaattagcctggcatggtggctcatgaccgtaggcccagctactcaggaggctaaagcagtagaatcccttgaaccctggaggcggatgttgtagtgagtggacatcccgccactgaactccagcctgggcaatagaccaagactttgagaaaaaacaaaacaaaacaaactagttaaaaaaaagaaaagaagtggccgggcacggcggctcatgcctgtaatcccagcactttgggaggccgaggcgggcggatcacgaggtcaggagatcgagaccatcctggctaacatggtgaaacctcgtctctactaaaaatacaaaaaattagccgggcgtggtggcggttgcctgtagtcccagctactcggaaggctgaggcaggagaatggcgtgaacccgggaggcggagcttgcagtgagcggatcgcgtcactgcacttcagcctgggagacagcgagactccatctaaaaaaaaaaaaaaaaaaaaaaaaaagaaatggggagggtagagggttccccattaacttatgctgaggatctagcaagtaggaaactcagatgataaataagccaatgcaatattttatggatctaaatgttttataacaaattcatccctgtgacatatttccatatagattttaacttttatgagatttgagagcacatcttatgtcacacacactttatcattacagtggcaacgcagcaccctgatcatcatagataatctgtgaattctttcacctggtagcagcatttttttaaatcctccttttataacatggttggactgggaagaagaatgtatctctcataattatcttctacttttattgtatgaatatgtaaagcagaaaaccttactatttcagtaaattcatacttgccactaaagtagaaagtaaactttatctacttaaaagaaatcgggaaaatacatatttttaatccaaggaatgctaaagctcgtacttgttccaattgttgggtgtttggggaaggacaggaattgtgtgtattgtaattatgactatcgaaactacagacttccatcagaatctctgttcccaatcgtagccagcattattctcatcctagatttgttgccagttgtgtaagggtcatgtggaggtgaacagaatatgaggtatctggctccaattccatttggaacattaaagtgactccagattgataaaatagagagagagaacagcatgcttgactacaaagatcctaagccagagtgagccaaatggtacattctcgactaacggtgactaacaatgaaagagagaagagatcttaggagtggaattgaagcttcattttatcagggcttactttaggatgtaggtagaataaatgagaatgtttgtcagaaagatagtgaggagtaaatgggcgtaaatagctgacacaaagataagaaagctgtcaacattttctgcaagggtatgaaaattcaccttttcagggtcacatgctggctgccagtgaaacgatggtaaaaaagagttaaggaagctaaaaacagaaactctagggatggcctcagaatgtggaagagaggaaattccactggatgtctatttgaaagattgacaagaggaaggatatgtaataaagaagtcaaaaaagatgaaaatggattaaaacttgaaaggattattaccgattccctgattttggtctaacaccattgcttaggcgccacacaagtattttacgtaaactatgaataaaatgcagttgtcatctatgttttacaaatgagaaaactgggatttaaaaaaattaacaacttgcctaaagtcatgttattgcttagaagggccaagtcaggacccaaatgcaggtttatttagttccacagcccttgctatagttaaccatcaggctgaaattccatcatttctcaaagcacctcctttctcctactctgatcaaacatgtattctctggcctgaacatgtaaaattgttttcaaattgctcatgcaagaaaattgctgaatatatttgttcatattcccctacataactgtgtatcatgctttattatatcagactgactagctctgcacaattggttttgcttaccccttagcacaatgccatatgtaaataaatactattactatagatgattatactcattagaaaacactcaaaggaacgagactgcagagattcctggaatcagaaaatttgtacatgtaaactagagacatatttcacttataaatagtgtgaaatttctttttccaaacttttccattcctcagatattaaaatcaaaagtgttccttatttgtttatgtgctaattcaaatacgtagctaaataattatattttaggaaatccaacaagatatcctgaggtaagcatatttttatttatttcagtgatgcagttcattcaatcatttccccctgcccttgtcaattgtttcagttgctcacaggcaattctattagaaataaaatattgttagagggggttctcaaaactgctttatcttcattatattgaaaattctattccaaacgaatctttggggaaatcttttttactcacatacttttctttcatagtttggtttcaaacacagagagttattcctgccaaagcctcatttttctccttcttttttggctaaaagcaatcttctggagcctgttcattcccctacccctccacagatctcaccatgatgactctcctaaggacacattagagggctttgccctaatccatattttactaaacagccccaggagtgagaagaaccttcactaggatttgattaaacaaattcttaaagccaataaaacgttttcaagttgtaagagaaagcagcattagtgcatgttcccaagcagggcagacatttcactgttgtcatccttaaggaagacagtttgggatgcagtacattctccctgagactcattccagaggcttcagcctccagcaacagttgcctgtgagggtagctttattgcatagcacaatgcagttgtgactgaagctgtttccaaacactttagggcaatttcatgtgtaacttttcctctgccactttacagggttcctgaaagggtagaagaaaatacagtaggtaaggccagacaaaacttgtgactaaattgaatctggcataaaataataatatgacatactcttgggacaaataaaaaagaaagctgcttgttaaatgcagtagattagaaatgtatatatgcagatgtgaacatttaaaagattaaaaatactggactgatatatggaaacaacttttatgattaaataagataagttatgaaaacaatcctattggatgttcaggaaatgtttaaaacgaaccaagtgggttgagatatagttggttcatggccatatttatatgtggtaaaatacccatactatacaatgaggttaagtctagcaattatgtggttaatttggtcactctccataatcatagagattcatttgttgggggtgggatcccctacaggcattggattctcccagttgactggtaataataacaaacaggcttaccagaaacaaacagttgtaaagtctccactatattacgtgttcaattgatgcagttgaaatatcaaacctagcttttcttttccctaatgtcagcaatgcagacaaataacaggcgtttggaaaaaagaggattttttttaactctgagatattttaattctgtcccagcagagagtcaaagaatcacaggaacaaaatatattattcatacaattttatatatatatatatgtatatgtatatatgtgtgtgtgtgtgtgtgtgtgtgtgtatatatatatatatgtatatatagtatatatacagagagagaaagagagtctgatctgttgcagtattggagccttgggaataaagtatctttttgggaatctctgtagtatattattagaagatcatgattaatgtcacaggacttaagcaatctttgaaaagaagtcaaaatgtgccacaaaaattcacttgattgtgccagtgggaatatttcctaacatagtcccaaacttcaaaaacagtataaagcatgcaaatggagtaaggggcggggtccatccatttcttcccaagtatatttaatctatttaacagaaaaatgttgggaaagttgtggctcttattagcatgagaaaagggcatccagtggtatctatacactagactgttcacatgttgtttttcccagggtaggggtggtattcgtacttcttattgctttatgtcaccagtctgtaacccagattgtaactttaagatgttatcttctggctgctcagataaacagcccattatgtttttatggtgggcagttctgaatcctcagggaattagaagatattggctaaagtttgtggctccctgctagactatcaaggggtccatgggatcttgtccggactatgactttaagcaaacacctctctcataatggtattctcacactgccttcttggccatggacttcaaaagctgattgcagggagggccagaaaggcaataacccttaatatagcaggactagcctagaggggacagacagggcaaaggtaaggcgtctctgaggttggcatttgtgtgtttagccacatggggtgattaagggccatctgttcttcagtgggctaaatcatcgttttcacagcatcccaggatcaagtacatttcatgcttagaaagcatgaagaagcaccctagagttttcccagtcctaatgagacatattttaaatttatttatctttgatttctaaataatgatagtatgcataggtgcattagtgtgtgtgcacctgtgtgcgcatgtatagatttgagttgtttttgtagacttgaaattaagagtatcacaattaaagcaaaattcttgtatgtattcatacatcttttggacagggaaaaaaacctctggaatgttatgctgatagaagtctttgttttgttttgaatatatcgtgtgaccagttcatttttttttaaattagagaaactccttgtttgaaatccatgtacagagaaatctatcatctttctgtgtgtgtattatcaagtctttcaaacaacactttatttcagaaaatgcacattatcaatttgtgagaatagaaatttgaatttttctgatagtatttccacactgaagataatttttttatattacaggtcacagatagtatgaagcttgttaaaagttagatgtgattagtgtggatatccatctttatccatactactagggctatataaccctagttatataacaaattacactgataatttgttaaataacaaattacattgaatagttcaaggcttatagatttttaaagactgtatcattaactcactgtattcacattatttataaggtaagctaaccaccatgtcttccaaagaaataattaatatactattaattttaactggactaaaccaaatgattaaaatctgtagattctgaatctataaaatttttgaagtacgatctatttaattgttacatagttgaaacttactattggcaatcgacagtcttccatcatgtcaacatctttaacatgatctagcctaggttgtcagattcttcatcaatctcagaaaagataataaaaaagaagacatcaagttcatgtttggctatagggagagcaagtaactttcagggaaaaaaaaaattataccagtacatcaggtgagtaaactcagtcctactaattactagttaccacataggagctcacttcaaatctaagcactacaagaaaagtgtcctctattgacaagacacccaaacagcatttttattaatagggacgacagtctagctgtcatccaaaagttatgttttcaatcagccaaaccatagcaaatatatcctaacattaaaacatgttttttcattaataacagtttgcttagtggagattccaaaacctaagccatactttggaaactttccctatgcaataacttcctaactcagcaatcttcatactttttggtagcataagcgtaacagaatttgaaaaactatataccttctttcgtcttttaaggggatgtctaatatttttatgagttagtatttcttaaaggatatacttttaagcatattgtgtaagtgatttaaaaacatttcctcaaaatattggacctctggatttagctgattcaatttatggaaaatgtccactatgcaatcagatagacaataccagtcttccttgtcacaccaatcaactaaatcagactcacttataaaaaaaaagtcttatcctttttttccattcaaatgttgtaaatgtgaattccaacataagcagactgatgatcaatggatagataagtaagtagatatatatacagtaaacaccctttctctgagtacataccctttctctgagtttgtaacttagataaaatgagatactgcctctctcaatatttctttataaaactcacagtattttgtggtcaaaggaaacgccttcagaaataatatgttctcttaattactctccttcacatacctcaaacactattaacatgaggccactcattatttttctgaaaatataacatgcttttagaatttttaaaatatttatgtaaaaaatttttagctatttgttccatttggctgtgtaccactcatctttttatgccatatacaagttaccttgtcagtgaggtctttactcaaccaggtaatctgatgtacttcttcatctatgttcccatagaatattattttacaaatatattgtcataattgtgtatgtgtctgtcttaatagactatgaatgcttccccaatgttaaaaactcaataaatgttttaaaacaaaacaaataaatcaatgaatgattcaatcattaagaaataattccaccaagaaatgtctaaaatggtgctttacaaggtcagttgccatgtatgactttatggtccagaggagatgattaaagaaatgtgtgaaatatattttactattcatgcatatacaaaaatgtatttgttcctgaagagattgacttatagggaacattttaaaagtgacagtaaaaaactgtgtctagaaagatcacacatggacacgatatttaaaagcattggatccatgcatggttccctggtggcaaagacccttagctgaaatggagggatttttttttttttttttaagtaaccatattttaaaagagcaattagatagagaaaacataacatttgttttataaccacttttaaaacttcattatactcattctacacatataatgagtagcaaataaaatctttaagtagactccaattcttaaaatattatgtagagaattatagcccattattcacccttttaggttgccaaatcaagcagtttagcaagatcttctccagtaaaacatggttgaattgatatgacctacaaatatgattattagatattctgcagaaaaaggtagcagagcaaacaggaaggatcagaaacctccgcatagcattctttaaacaatttttagtaaattgtaacactgcattttaggagtcacattatttttatttattatcttatagctatattctaagaaaacaaaggaaaaagttataaagtcctagagttttgacaaatcataaattcttccaagtctttgtttagattttcgagaattattcaataagcgatcatttgaaaggctttattaccttttcttcaaaagtcatctttaaatcagatgtacacatttcttaaccacactctgttacaaataattatgtagaaaaatgtcagtaaagctatttctgaaaaattcagtcagaccagattttttcagccattattttccccaaatttaattaaattttttttctatttttaaaatttattatattcaggaaattcaaggaagatataggttgtggttttaaactagaaaaaatgagcatatgcatactctagacttaatgaatacatattgctaaatcctttacaatgtcattgtatgcagaagtagttcaaaacaccttatttttctgtgttcatgtttatggtctcaggaattgagaatgaattttcctcagtggaaaatttagagtttatttaccttcactatttctccatagatacccattgtagtaaaagtttcaacagtggaatttgcagtgtgaaggaattgggtctgagacaatgttcttgctagttctctaacaagccaggtccacaggagtggcgtgtgacccgatgtcactatctttaacaaaatggcttttgcacaaaaagaaagacctttatacctaaaaataaaatcttataacacattgttaaaattatttagtctggcatggagttttattaagctttgtgttattcatgagggacaaagaagatcatgcccaagaatgaaaaagaaaacacttaatggggtctgggcagttttaacagcataagtgaaatacaacaccaaacaggatgtctctcttcctttgaacttgaggcattccatagaccctaagctactgaattctctggttagttatgtggtgccagacattcagtggcacttaatgaagataagtttctaccttgtgcttttaaaggtaatggtgaatgaatcctgcctgaccaaattgagtgtttcttaaaagttactgtaaagttggaaaaaatatacatatttttcttggctcttaaagtgattagtctcttttctatgtttttatgatcaaatgctaataaatcttcaaattagtaagcagaaatatttctatatttttatcttaagcataaatataaatatttgtccatttttgaaacataaataagactctaatggaaaataaaatttacatttaaacgtcaacagatcatatttcataaaatagtttcttccttcataaataacacttggaatttaatgtacattaggaaaagtatcttcttgatgtttcccttcggcattaatgaaaactgcagctgctcctttttgagattttcttgattatccaaataaacaaatgttttctttatgcttgtatagtcaaggaatacaaaatccacacctaagaaactgctacctctcccttttggaaatatgtccatttaaaaagtggttaatcatgattaaataatgacttattgttactaagctgcatttcaagtctctaaacaggaaactcttggaaattgagtataacaagaagcttaaagcctcagatcaaatgcgaactccaactgtctaaccttacaagagaatagacagccaaagagagctgttcgatgctaagggaaacatgctgccctgctgttttttatttttaaatctcagcattaactgaaagtatcaagtcaaaactttcttctttcataaaaagataacactcatattcaaaggaggagtacactcacctaataagaatttaaagtgactcacttcatgagctgatctttagaataggatttagtgactcacttttgtaatcatgctctcgtccttttgattaacaaaaatcagaactctttcatactatcaattccaagcatcctcctctcttattatcaccttctagcttttcaatttactctctttactgccacactgcagtatttctaggatctacaatccattgatcctaccaatttttcatttctcttataatctacattgtctcactttccttttaaaccagtttaaaatccttagtacatcagataatcacttgtctcttttactctaactcaatcctggttacatccagctctctgcctagcggggcctgagccctatcgaatatggctggtgaaatggtattgttatttgacatagttattctgactggtctcactttaaagttatatgtgaaatttacatgggctcataagttgttctaagctattttccgctagtagatcccctttttcactcctgtagataattatgccataccttctctcatgttttcaagcctccaaaatgttcttgcccattcgaattctcagtgattacctttgttcctgtttcactgagaaaaaagtcagaagcacacttcatatatctcccaccattacacccatcacctgccagcatctggacccacatactctgctttttcaactgctcctgtggataaattctcctgtatctaagcccaagccttctacctgtgtcctagaactcaattctttcacaaattcaagaacatttgtatagcaattctctcttctttttacagcagcatcaattttccctctctactagaagatgaccagcatcacataaatatgctgtcattttattaagattattttttcaagtactcatcagaccctttcctccctctacctactgctccattgatctcttccccgttagaataaaattcctcaagagcagcagtctatatgtgcagtctacaatttctctcctccaatttctcctctaatcaggcttttaactcatcaaactccttatcttgtctaggtcagaagtggcctgcatgttactgaatctagtggtcagtttttatcttacttgaccctttaaggacatttgatagagctaatggcttgtgctcctctttgaacggcttgcctccattgggctacaggacagcacactctgccagtgaaaatcaatcaggcttcatatggctccccctcatggctccaatgtcataatgttagagtgtcccaagcaacagtctttgcatctcttttctacctacacttgttccctaggtgatctcatctgggcttacggctttaaatagcgtctatatgctgataacaactaaatttaactctcagcttaaacctttttccacatctttccccatttcagttcagagccactcattcctctctggtgcccagacccaaaaccctgaagtcatcctttactccattcccaccttctgatcttctctcatacccaatccagcctgccagcaaatccagttcactcacctttaaaataaatcaaactatgactacttttccccacttctatcacccttttctcttgaacatctcatacttgccttcttcttcctttggcactgtgggcttggtcctgcctcagggctattgccctttctgttccccatgcctacaatgttcctccctatgatagtttcacagcttgctcctttatcaccttcagctcttcaggcaaacatcatttataagtgaggccatttctgatcacccttttaaaaatcacaaacctcccttgtcccagcaaaatctgaccctttccctgcgttcattttattcatgaactcctaatgtactctatgtttgcttacttattttgcttaggctttaaccactagacaatgctccccaaaagaactttcagtgacgatgcaaatgttctatatctgcaatgtccattgtggaagctgctagctgtgaatggctattgggcagttgaaatacatagtttcattaagttaaatttaaataaccatatggccagggagtaccattttagacagcacagtttaaatataagccacatgcaaacagggagttttgacttcttcagactgatgtagctccagcactagatgcctgatgtatacctgtgaattgaattagtcactttcttttctttctggttttatttctctggttgaatattgccccaggccatggtatttggttgataaggagagcgaaggttatgttatgttccttcacgctgctgccccttgtgtaaagcacaaactacacaactacaggttgtgaccctgagtaacttggttttgcagatctccctgtagcttacggtttacagctttctctctgctttttgacactactgtcaccatcaatatgaaaagaacgttaggggtacagtgagagagctctagatatgaaggtgtttgcatgtctgttcttctcactgctgtaatgacaaaagcacttagggttcttttcttttctttttttttttttttttttttcggcctattacctacttgaaacactgtataagctctcaacacagctgcagaaggaaggccaaatatgagaagcaacaagtaacaacctgggaggaaaaaaaaatgatctgacataaacttaagttccttaaactcagtattaattaatgctagaatataaatcacatctctctatcaagaaagaagtttcatcagtcttacatggggtagatttaattgcattttatgtctgggacaaaaacaaatataccttcctgagccttcagaatttatttctgggcaataattctccttttcccactttgtattgcttcttaaaaattatcctttaataaagcatcataagagaatagatacacgaggaaccaaatttacctctttccgtctttgcagggcagggctcaagaccttacagtggaagagttatgctctccataaacaatatgaccttccaggagagaagaaagaatagcggtaaggacagagagggagagagactgcctctctttgttttgaaggtcaatttctgatataaatgtagacagaaagtatattccactagctctgatgccagaccacctgtgaattccatctctagctctttcataaatttgaccctttcttatcttttttatgctttagtttcttcatctataaaatgaggatcatgttgatattgtttatcttatagcattgttataaagattaagtagtaaatgcaatgtgcttactattgattataaacacgttttaaatgttcgtagctcttgatattctagatagagaattttaaaccattgtatgagttggtccaaatacttcattttccagatgacgactgaagacatcaattcttcaaatagatacttcgtgtctgtagaacgcaggtctcctatttcctagtttgtgtttcttccaataaactccttgaaagctcactatttcccctcattctttttcattcttttcttgattgttacatgggagtaaatgaagtaatcagagcttggaaataagtggtaattgtgcctttggttgcaagtaaaaaattacttattttactctccaagatttattttttatttttattttttacctacatgggtgtcatcaaatagataagctggatctaatcaaatagatcacccccagagggaatgaatccattcatacaaagacctgccaggctgtttgacactagtatctctgtcctatgtgtcctatgtggaaggtgggtctagggctgcccttaaatagatgtgaatagtgtagacttttcaggttccaagttgatgaaacttccaaacctttggtgatctattgggattgaaagttgattacaactttcccattgctaaatatctgaacaccacctcaagtcagtcacacaaggagctagggccccaagcaccctggaaagactaagaagccagacttgccaccatccttaggctattactttggcctggtacttactagctcttaagcctggtactcaatgatttactgttcaagtgcctgcagtggttcttcctaattgctgatctcattttgcacccaagtgccaaatagtcccctggagactactgagtaaaacaacaggaaaagttctaataaccatcaggatccttagtaaaatgcagctcttaaagtagaggaccttcctggaaccatcccaagtagcctgacactcctgttttctttaggcacttcatgtgcttgtcagttactgaaattaactacatttcataaatgattccctctgctggctacttgggcgacaaagtggttttagggactcactttatttacatgattaccctaaggcaggaaggaaatattgtgcataaggaaagtgctgtggggagaaatctttgactaaagagtaacaacatctgagaaaactgtttatagtagatccagaaaagtgcaaacaaatgaacagagctggcagctcattctgaatctgagataagcaattctaaatgagacagctggattttatatagcgcacaaatacgtgctatgaaacccagatcagacagactcgccaaaatatcctagactgctggaggtcagtacaggtcagctaaataaattaaatcctgagtacactaccttggtctaattgcctggtctctctgggcttacctttgtcttttcatcactagctccacctatcctttcatctattttctactttcgcttaaagtttatattacggtgccatcaaagataagtttctatgttacagccccaaagtaaatattaatacttctatgttacactgtagtgaatttcctttttagtttaaaaaatctgcttttcaaaaggtaatcgaccacaatggatgtggtataattccaatcatcaacagaaacgttccctctgtttcattctttgtatttatcttgagcagcataaactcttctttcccacatatgaaaatctttgaaatgtttatggaaatgcttactaaatgaagaagtttgctaaatgtcccaaatattattaaacctgtactatttacgtgtgcttggctatcaaaactgactccttgttacatagatgtctgggagagtcatcctatgggcagatctccaaaactctccaaaacaaatctccaaaactctatgactcgtgtattgtagacttttggtggttgctcattacagcagttttcatctgttaggtggtttgaagcatttaaaagtcagatagttatatgctgttatcaaaacacagatctagaactgttcaactgtcatgcattaagagttgctatgcaggcattaaagatgttataatccagagctcttttgaggcacatggacatgataagactatctaatattggctaacatttgttgagtgcttgctaaatgtcaggctttaggctgagatgtttaaatgagtttctgtatttactcttcataaatctatgagatatagtcctatttgcatatgaggaaattaaggcccactaaaagggaaagtaagttgcccaaggtcacgcaattagaaaagtggcagtggccagtggcggtggctcacgcctgtaatcccaacactttggaaggctaagaggctcacttgagcacaggagttcaagaccagcctgggcaacataacaagatcctacctctacaaaaaattttttacaggaaaatgagttgggcatggtatcacaggccggtagtgctagctatgtagaaggctgaggtgggaggatcacttgagcccaggagctaaaggctgcaatgaaccataattgcaccactgcacttcagcctgggtgacagagcaagacactatctcaacgacaaaacaaagaagaaaagtggcagacccatgtgttggtcctataaaggtgtatatactgtggagcctgctttttgaaccactgccaagtactggtctggcaggctgatcattgtttccttttcttttttttatagcaacaagcacaatacaacatggatgcttattatgaaaatatcttgaataaacacccatgcagcaaacttttcaagtaaaagaaaaaataaagagattgactttaaatatatttttaaatagatcatttttttttaaatcactccatacatgaaagtcaataaatatttctggagcctagacaatagctgaggtgatattttagcccctggggacacatagtagttaacaacacacagtaacgtcaaagaagtgatagagcatgatggaggatgggtacactggtcaggaaaattgacctgagaaggtgatatctgagctgagaagataaccaggttatcactaaagaagagatcctttgaataggatcttttcatgcaaagaccctgatgtgagcctggtctgttagagagacagaaagattttccaggctgcagaatagtgaaaaaagggttggcagaggagcatagagtagggtagagagtacatagacatcacgtgtggtaagaggtgtacattattgtagttgcaggaaaagaatataaagcagaggaatgatacgccacagatagtcaattgtttggtaaaataatttctgaaataggtaatttctgtagcttatagacaaatttcaccaaggcagagaaaagaggaaaacagcatggaataaagaccttatctatagactctattgttatatatgactagacaaagtcgttcctgaaaaagggttgaacattacacaagcatgcagcaatcaaagccagtatttacaaaattcatatttataaccctaggcatttctttttcacttctatcattaaaataaatatgtattttacacaggtggctcatttagactaaaaagttacattaaccatgtaaagagatgataggaggagagaaataatagttttaatctggctagatccacacaatttctcatggaagaagctgatactaagatctggggatagtgttaaggtgggaggtcgataggatatagacacttgtaagacaagttttgcattttaaaaacaacttggaggaaaattaatacatctctattatgtcacttttcctattgtttattgaaaacgtatcaaatcctgagagccagtgaaagctcatttcttattttattttattttttgagacggagtctcgctctgtcgcccaggctggagtgcagtgacacgctctcggctcactgcaagctccgcctcccgggttcacgccattttcctgcctcagcctcccgagtagctgggactacaggcgcccgccaccacgcctggctaattttatttttttatttttagtagagacggggtttcaccgtgttagccaggatggtctcgatctcctcacctcgtgatccgcctgcctcggcctcccaaagtgctgggattacaggcgggagccaccgcacccggcccagtgaaagctcatttctatagagctctttctattaatactgacagatcaggaagaatttatggcgctttaccaagtaacagttaggtgacttgggataagataaatgagggagttcataaaagttactcttggtctgaaactacgtgggagaaatattgggtaaaggtggctaaatttctatcattctgaggaattctaagagctgttggcatgagaaccactgcaactcctgatgacttctctgtggccacaaatcttggcgttgcatgggacaagtctagaggttcagttggaaagggattaggtagaggaagaaaatatatgtggaatcctagccacttcctataaaaaccaacaaaaataactaaatatttcaaatgtttttagaaatataaaataattatgttaaacatttaaaaataactaaatagcttagtattaattttcacataagattccaaatgtattttgacatatgaattctgaccccagcttatatgtgtgaaaacaatagtagatttcgtggggatgatatcatttatacgttattatatgtagagtgtcacattttgcaaagcattttatttaggtccattacctcatttgattctcataatggccctatatttagccaaagcacacgtgatttgtactatttctgttaggaataaaaagaacatcaagacacaaaaaaagaagttagttggtagccaattagacccaaactgcagatcaaccgattccaaatcctgtttcctttttttcaatcactagcccagtggttctcaaagcctggtccctagatcaggagtatcagcatcacctgagagcttgttggaaatgccaattgcagtaccctctccagacctactaagtcagaaattctggaagcggaacccagcatgcctaacagggcctccaggtgatactgatgcttgttaatttggaaacctctgtagtagtccatagtgactctccagcccatgacagtagtgtgaggagaacttcctatagagggaagctgcacgtccattccccaagatttccttgattacttcatcagccacacacgtattaatagctgattcttggtcacatactattccatacacttaacactgctggcaccaggccatgacattacttaacaaaggatttgaccaactgattcttttccgtattgatttgatttgtaagatgaatcataagccaattattttacttatagcaatcatcataataaagggtcaaaatctacggttatggagatgtcaggccaaggaagaaccattttttgtctgagctcaaagcaaactcacagtaagagaatcaactattatttaaaatttgtccctggcaatattttttgttcattttgttattttaatttttgcttctttgggattctattcagtcactggaagtctgtaaagaaaatataaaatagaaatctaagctattagattaggaaggccattggctgggattttacaaacatctcaaagagtaaaatattattttccattgaatctaagatgccatcaattgtggcacaaaataaatgtaaccattattttatataccactatgaaaattaaaaaaacagcctcaatctcatgatgaaacaccatcaagtgtgagagacattccaatttcagatatgtcaaaatatgaaaaagtatatcttagtattaatggaatacagtacacattagctgcagttggcatcttcctctggggcaaactctgcttacatattttcttttcataagcaatgttgaaatggcctgttatggtacagttatagtaactgtgttcttcaaagttaagcagcagtcatcctagcctaaggattgtttcatgacatcagtaactaaacagtgcaggtggaaaatattcttctacaatttggtatctgagttatcatgtggtatgaggaaataggcaattgagcaaggaagtgaatgcagagtaagagcgaacatggagcactcattcacttgaataaatcttttgattgtaatttaaaaataaaatcattctcaaatttgtgtttttatatcctgacaggtttgccttcttaatcagccatgttgctttttttgttagctgttaaatatatatgagtgaactctattacatgcaatcataaatgtcttgcatattcccttcctctgtccttgcaggaagccatgtacttgcttgagaatataacaaagagacatactggtttgcaggaattttgagtcaaagagtaagtccttgattcgttcttgtgaaaaatgctccatgagaaataggaaacatggtctgtaaactgctattatattataaagcttacttttctgacctggaaaattttatttacacaaaaaagtcattgttggaaaaattttcttttcgaaaaatattttcacaattcagagaagcttctatagtaattaaaaagtgcgtacatatttatttgagatatttttaataacccattaaacctagtgaaaataaatgaaggcggaagtctagagacatgagatctggttgtggttttgttagtcttcatctctgtgacttaagcatagttacttaatttttctgttacttttaaaattagaaatataaacggttgtaaaattttgtaatggtttcagagggatgagaacatcttaaaaaatgatattaatacaaaaaatacacaaaggtactatgtatatattatctcatttaatcataaaaacaaaataagatagtgcagagaagcattgaggcatagtgcctagtgcatggaaaacgttcaacatcactggcaggtattataattctatcatcaataacaacaacacagccaggatgaaattaatgttcccttttcagtcaagaatataatcttcaagaggttgggtggcattcctgggataattctgatggcccaggccagaattatgatgttttctgctggacgaccaggctgtgctttcacccttacgaccacattgccctccgcagtccttgataacttaagcagaatgtttcgagcagaaagtcatcttttctatttaaattgtaattcccaacgtattgcttgaaaacacctcaaaatggtattttacatatctactgcatgacttttgacctgcttttcccctaaagtttggattaaacttgaagaatatcagtatacagaaccaccctttctgccagttttaactggaaccgaagaggtgtgatatacagagtattaaacagtaaagagaggaggagagatttgttgtgtgggtgtgtgcatgtgtattgagaaacagggatgtggactgaagtttgaggaataggtaaggaaggtcgaaggcattctttccttattttcctgcctcccttcatgttttcaagtgctacatactaaagaagaaacagaagccccaactgactaaaaacatcagcctaaggtaactttaacacacatgcagagggagacttgtaaaaggatgttcacttcaacattgtttataatagtaactatagccataaatcttttctatgttttcttctattaatgttacaatttcaggtcttacacataaatctttgctccattttgagttgattttttttacatggtataagacgagggtctaatttcattcctctgcatttggatatctagttttcccagcaccaattatcaaagactgtcttttcctcatggagtgttctcgccatcgttgtcaaagatcaattgaccatggtgtatggatttatttctgggtcctctattctgttccattggtctatgtgtctgtttttatgccactgtcatgcttttatgattgctacagtttcacagtagctgttaacattggaaagtatggtacctccagctttgtgttttttgatcaagattgcttaggctattcagggtcttttgtggttccacacaaatttttgggttgattctgctatttctgtgaaaaatgtcattgggaatttgacagagattgaattgaatctgaagatagctttgggcactatgtaaactttaacaatgttcattcttccaatttaggaacaggggatatctctccatttacttacatcttcttcaatatttttcatcaacattttatagttttcagtttggagatctttcacctccttggttaaatgtattcctaaggtttttgcattttttcctttttgtagctattgtacatgggattattttcttcatcatttttcagataggtcattgttagtatacagaaatgctattgatttttgtatgttaatattatattctgcaagtttactgtatttaattattggttttatcaggttttttttgctggaaccttttggattttcaatatataaaatcatgtcatttggaaacagagacagtttaacttctccctttccaatttggatgcccttcatttctttttcttgtctaatttctctggatagaacttctgttagtatgctgaatagaagtggcgagagtgagcatccttatcttgttcctgaccttaggaaaaaaacttttattttttcaccattgagtatgatgtatttatagccttatcatatatggcttttattttgttgaggtacattccttccatacctaatttgttgggagtttttattataaaaggacattgaatttgtcaaatgccttttctgcatctattgaagtgatcatatagtttttgtccttcatactgttaatcaggtataacacatttgtggatttgcctatgttgaatcatctttgcatcccatagataaatcccacttgatcatggtgaatgaaccttttaatgtgtttttgaatttgatttggtagtattttgttgagacgtttttttctgactctcaagtgtgtttcagatagttaactgtcagttaactaaaattgtagtcaattgctaaaaaagcatcactggactatttattctgcattggcatattcataatgttaagagcagaacatacctcaatgtatcataacaaaatgcacagtttttaggcaagcaatgactgaggtatcctcatcacaaaataataatgctttattttcttttaaaaacattaccactttttcaattgtctctggatttattataaagtaggaatacaaacagatatagaaatgtgaaatgcaggcacttattttgctaaactggcttttaattaaatggacaatacacttactatttacctaaaatcctgcattgcttccaaagatgtctgcccatcctccttccattttcttcaaacaggagtctgaaacaccttcaaaaaagctacagaatttgtgttgtatattttgtgctcaaatatatcatctaaacacccagactttcactgaatatttagaattgttgaaatgatatgaacaataaaattcaaaactattaattcacaacctaatcatttattacataggttgtaggtaaaattattatctcccttccccacccactttttttttttttggctgattaggaaactaacttgattacaaaattagtgagaaacatatttgagatccgaacaaatattttccaataccaaaattagctattgtttactctcctttaattcttactgtttatttccagtttaataattaaaggaccctatcatctcccatgtcctggttttctgagcagagattaaatgagtttctcaccaattagattcaagcaggtgttaacagctgggtctccgcacttttgcccaaggcccactgttacaaggagaccatgacttccctgaatacaattcctattaaagggaaatattaaacaagtgtccattgtcagaagcagttttcagaaaaacaaggttttgttaattaaaatattatagaacacaggaatatgtgattcaaaactaagaaatggcagtgatggggaagatagcaatggcaaaaaagaaaaaaaattatgaactcctatttcaagaaacatcgaatatagtggaagaaatcatctgacttactttaaaaaaatcatggattctttttagccttactcgtttttaaaaatgctatttagactatgttccaggcacttggccagtttttgaaacacggcacagaagcagatgaaagaggttaatctgatggtagctggataagacaatacttcgaagaattaatgctgcatagtattcccctgtgttcaccctaatctaaaagaactgaagcccttcaagtttaaggttgacccttcatgaagtggtacctggtgggtaggtgacttctagacactccttttctccatacatgtttgctgtggcctgaaatgccattatgagaagacaaggcattgagtccctgattacagataagtaataaaagatacaactgctaaacagagctactacgttctgaatagttacagaaatattacaaccataacattaggtgaaatacacttacatttaaagaccatagtcaggtaattagtgaagcatttaagtaaatatgattaggttctttatagattttgataatgtggaaaaaattagacacttaggagagccatggacattttaagataggtatagagtcattagtaaaaagtcattagtaaaaaagaacaagaaaaaaagcagtcaacttcaccaaaacacacctagttctggctcaatctatattcaactctgtggacacaagaaatggaaaagtggagggtatttgagctacatacccatatatgaccttttattgagtatctgcaccttgataaagaataaatatccattgagaagagaggaccttagaatccaaacagattgtaatcaaggctatctaaaaaagtttatatgtctcatagatgaggaaactaatttatcttatcctctgtaataactggagttaaaactgaagtgcttattcagaacttgtagttagataaaacttcatgaggcacttgggataaagcatgtacacgcattgttagcagaagacgtagacaacatggccttgtgtatttatcagtttgtttcttggcatttgtttatcggattagtactcaagtttttagagtcttacttttggatcattgactgactaaacaaggaatctttgtacttccctaagaataactcactgttaagtctaaattcatttatacaggtatcttcccccaaattatgctttctcattgctaaacaggcactcggtgggggaaagagaatcagaagatactgaaaagaaatacaattttctgtttaaaaaaaagatggtaattcaagatgctaagctttgttatttggttcccccccccccggcaactattaactattcttttttaaatcacttaattttttttttaacttttacatttgggggtacatgtgaaggtttgttacataggtaaattcatgtcacaggtttttattgtacagattatttcatcacttaggaattaagcccaatagttatcttttaagttcttcttcctccttccaccctcctctctcaagtagaccccagtatctgttatttccttctctgtgttcagaagttcatcatttagctcccaattgaaagtgagaacacacagtatttggttttacattctggcattagtttgctgaggataatagcctccagctccatccatgattccacaaaagacatgagcttgttcttttttatggctgcatagtatggtgtatatgtaccacattttctttgttcagtcaatcattgatgggcacttaagttgattccaggtcactgctattgtgaatagtgctgcagtgaacatttgcgagcatgtgtctttatggtcaaatgatttatattcctctgggtatatgcccagtaatgggattgctggatcaaatggtggttgtacttttagctctttgaggaattgccacactgctttccacaatggctgaactaatttacattcccaccaacagtgtctaagtgttcctttttctccgcaaccttgccagcacctgttattttttgactttttcttaatagccattctgacctgtgtgagatggtatctcattgtggctttgatcgcatttctctaatgatcagtgatattgagccttttttcatatgcttgttggctgcatatatgtcttcttttgaaaaatgtctgttcatgtcctttccccacgttttaatggggttgtttttctcttgtaaatttgtttaagttccttatagatgctgaatattagacctttgtcagatgcatagtttgcaaaaatttactcccatgctgtaggctgtctgttaaccctgttcatagtttcttttgctgtgcagaggctatttagcttaataagatctcacttgtcaatttttgcttttgttgggattgcttttgtgtctttgtcataaaatctttaccaatacctatgtccaggacggtattgcctaggatgtcttctagggtttttatagttttgggttttatatttaagtctttaatccatctcgagttgatttttgtgtatggtgtaaggaaggggtccagcttcagtcttctgcatgtggctagcgagttatctcagtaccatttattgaatagggagtcttctccccattgcttgtttctggcagttttgtcaaagatcagttagtcataggtgtgtggccttatttctgggttctctattctgttccattagtctatgtgcctgtttttgtaccagtaccatgctgttttggctactgtagcctggaagtatagtttgaagttgggtaacatgatgcctccagctatgctcttttcgcttaggattgccttagctatttgggctctttttttggtttcatatggattttaaaatagttttcttctagttctgtgaagaatgtcattggtagtttgatagaaatagcattgaatctgtaaattgctttgggcagtgcggccattttaattatattgattcttcctgtctatgaccacgggatgattttccatttgtttatgtttcctctgatttctttgagcagagttttgtaattctcattgtagagatgtttcacctccctgattagttgtattcctaggtgttttattctttctgtgacagttgtgaatgggattgcctttctcatttggctctaggcttaactgttgttggtgtgtaggaatgctactgacttttgtgcattaattttgcatcttgaaactttgatgaagttgtttgtcagccaaaggagcttttgggccaagactgtggggttttctaactatagaatcatgttgtctgcaaacagggagtttgacttcctctcttcctatttggatgccctttatttccttctcttgcctgattgctctggctaggatttctaaagtgtgttgaataggagtggtagagagaaggcatctttgtcttatgccagttttcaaggggaatgcttccaacctttgcccattcagtataatgttggctgtgggtttggcatagatgggtcttattattttgagatatgttccttcaatacctagtttatttagagttgttatcaagaaggggcgttgaattttatcgaaagccttttctatgtctattgagatactcatgtgttttttgtctttagctctgtttatgtgatgaatcacatttattgatttacttgtcaaaataacccttgtatatttgctgagagaaatggtttctgtctcaaggactataaaatgtttattaaaggaatgtatttcacatgtttataaaaggaacatactttttatcagatatggctatcatctcacaatgattttgcataattccaaaggaaaaacatattcttgttcacatgacaaaagcaatttgaatatatgaattcactgcctgacaccaacaaaagcagtgaacgcatgtatgtgttgaaagggaagaaaaaatatatgttgtggcaaaacgaatttaaaccaaagaagcttttttttaaaaaaaaatgttaacaacactataaagccagggggtggtagtatatgatgaattaattttattgtttgaatacagcacttagttgtcatggcaacctgattaagctgtaacctgaagacaactatatctgaatcagcatatttagaaagataaactgataacttcaaggtacagtttgatgctgggtttagggcaggttaaaaagctatttagtcatttcgggttgggatttagccttggtccttaaagagcagcactgatactaagtaagtaattcagattgttgattaaaacttcagcagaaaccaatgccaaaagttttgtagtttcataacagaaaacacttaatatataaagaaaggagaggagaaaagaggaagaaagaaaaacatcactagtgaaaagaattacctttaattttaaatcacctctatatttgggctgagatattggtgattacaccaaccttttattattatttttcaatcaggtaacaatgtttaaaaacaaacaaaaaccctagttattttgacttcatttctcttttcctttcagagatagcaaacaaaactattttaaaagattgactcaatgtgctaagaaaatataattttagcatctctgacatggatagcatcctcaaaaccactctctgtaattatcaaatattctacttggagcaggaatgagttttgctagaattggaaaacacggggagaagaacttggttgtgtatcttgttcctactttttactttagaatctagcccatttctaacttcgttttgggatcaccctactcccacaataaaggatgacttcctgaacaagacagtgaaagtccagtgttaattgcctgtacagaacttttcgaccaaagcaatatgaatgcatctgccaggtggttagaaagcaaacaaagataccaagtggggagtgttttagggaacaactattgagctatctagtaatcccagcttctacccacttgttggggcagcatctctagaaagtatagctgagaaactcaggcttccatgaaataatatatacagttgcccagatgtgaggctttgttgtgtttcacttaagtatcacaaaactagtcaatgtctgtcatagactaagtttgggggattaagggtcatgggcactaatatgttctctgtagtgtgcatcgaaattctctattctcaactgggtgtggtggctcatgcttgtaatcccagcactttgggaggccaaggcaggtgcatcacctgaggtcaggagttcgagaccagcctgaccaacatgatgaaaccctgtctctactaaaaatacaaaatgagccaggcatggtggcacatgcctgtaatcccagctactagggaggctgagggaggggaactgcttaaacccgggaggcagaggttgtagtgagccgagatcatcccactgtgctccagcctggacaacaagagtgaaactctgtcttaaaaaaaaaaaaaaaatctattctcatccattttatgaaacattttctttttaatggaaaaaatacagaaaatgcctcatgcaatttcacctaactactgattcaggataagaactttgattttaaccccagttatttcagattttaaaataaattagtttcctcatctataagatatgtgaagagtttcaactacaatctgtttagactctaaggacttctcttctcattgtatatatttttccttattaaactgcaaatacgcaaaaggtaaagtatgatatcacttaaaatatgtctgcttgattttcctaggaaataggcacctagcagagaacatatggttcttgagtaggaaagatacacaaggggtatggagggactgggaacgaggggaggggagttggaggcattctagacaaactgattctgagatggaacacaatgaagagcttcctctccaatgaatagatatatgtaaatgggtaaagaatatattgaagatgctattatactctactaaacctagaagtggtagaacccacgtctcctatacctaatggccaaaagaaaccatgcgactattttcaaggtaacatttgagatttgaacttgacttggccaaaaatgaagacacaagggaacaaaatgatcaatccctttaactagttctgacgaagagtcttgttacaacctttccctgtggcaaggagaaagactaaacatagacactcctctcttctgagtgagattcttggatttaatggcaaggaacaacttttccagagttccttctttcttcgctaggtattttgtatttttttgggtttatctcaacttgatgtacaaactctctttgtctttgtttatttagacaactgtttaccagttctgacccacaagtgaggtttatctgcgatggaaagactgacttccattgagttgagttccatggataataataacaattttttaaaaaggccatacagacatttaatctcagcaaacacctctctattcaattttaccaaaatcatgaattattttgctttgtttgtctttgtagagtttcctgttttaatatgtttttaatagacaacaaggcacaaagaatcctgggtaaataatacatgttaaaaatagttactacctaacaatgttactagaacaattagagtgcagaaaactctttcacatgagaagccagctaaaaacaaaggttggcatgacgctatttctttgattctttttaaattaatacagcacatttgctccaaactcatctctcccacttaaaagtatttgggttttttgttgttgtctgctttgcaatgctccctaaaatacaccactgtgggccctattctttgtgtaattttacttttttcttctatttttaaatagatacccctggtgccaatgtacatttatatgtgagctatatatatataccttttttcccactcagagagcaagggaattatttttaaagaaatcctctttctagccagattccattaggttgtattattcaaatgcactgtaacataaaatttatttcttcatgtggtgtccagttgctaaggaacacagccaaaaagtccaactttgcaacacaacagaagacgagtagagattatgagggttgcccatagacacatacagaggctctgagcaaggggaattacacttttgttttcaaacttggaagtgcataatattattaagaaatagttatttttatcccatgtacaatggaacttccattctccctggaaaagcacagtagcttttctagactcatgacctctcaagtcatgcagcatattttaaacagaagccctatcatttctgcctttaaatgtaaaacaggtctcctgaaaagcatacgatttctgaaatgtgctgtcatgtccaccaaacaaagcactttaactttgtttttcttaaaggcaaatttctacagaaacatgaatccgacagagcaataaacaccacctgaatcattttcaaatgtctcacccaagtttactggacacatgtaaaaggtggtgtctatcataaagacctaaaagatggtatgatatcagagcctttcgtaaagtcctgaaagaggtacttacattttgccgtcactttgtaccctcctagaggaggggtgtggccttccaatgcatcaaatccagcagatactaagaccatgtctggatcaaactctttggccacaggcttcacgatggtcctgcacagtaaaaatgcaaccggtcacacgtgggactggtgaatacctttaaaaatcgttcagtgaaaacgacctggcacaacaataggaacctcccaagaataaagaaaaggggaataaaaacattatttgaatgggacaaaagcgtatcatttccttaataataaatcgctgttattttagcatcccaattcagtcttttttggttcatcttttttcctccaaatggaaaaaaaaaaaaaactgctttgcaggtacattctaaagcccatcatattacacatacgtctgtggctgttttatcccactgcctataaaaaactgcttcttccaagggagtgactaatgttttacagaggaaagtcaaagctttagtgaaaacccggcttgctccagttagtgagaacttggtgtaaatttgaactataaatttcttttgggaaaagtttgctttcccaacttaaaaaaaaaaaaaacatttttaaatatttatcatggtagggggtgactcttgcaaaggagaaacacaaagttcccttaatagaatccagactacaatgttaagt

SEQ ID NO: 36 - DNA, 122186 bp, Homo sapiens
ggatcccaaatatctcagagctggtaggacctggggtttgaatactgacctttgacacaatgcggaagagtctacgtgactcagagatcacgttggtcccagaaggaaaataaggaaaataagcctggccaccctggataggggtaggttgttgggcctcaaagaggtttgcctgagcaagagtggctcaggcctgggcaggccactgtccccaggagcaccctccctgcccatcgcgtccctctgcccctccctcctgcacatgtcacactgaccacatctgtagacatcttgagttgtagctgcagatggggaccagtggctcccattttcattttagccattttgtctcctgcacccactcccttcatacaatctagtcagaatagcacttctagggcacacgttctcagtccaagctgtgggaaagctccccttatccaagagagtttaaaggtagtgacttgggtttttgcgagtgttttgtttagtaaggacttgtggggaggaaccgtgctaagccataaccaatgaggagaagcaagacagcctgtctgcccccaggagccagtcctctgctcttctgcagtcaggccactgccttggggctctagtcattccagtggaagatgaatgtaacctgcctggtgacgtgacaaccgtttcctccctgaccccagaggagctggctctagaaggttgggatcaatcctgaatttagtttatgtgttagatttatatatatatatatatataaaatatatattacatacataatatatataaaatacatattacatatatgtaaaatacaaaacaataacctttctggggtttctcgtggcagttgaaatagtccctcatgtggtcgtcagaaaataagccattcctcatactaatatgggataagctccttgacctctgaggagcaggagtgcttcctgctgtgtgttttagaatccctccccgccttgtttcgtggcagtgaaatgcctcttggtcctgtccaaatgtgtctttcactgattttttgaatcatgttctagttgcttggtcctgccacatgggtccagtgttcatttgagcataactgtactaaatcctttttccagatcagtataataaaggagtgatgtgcaattaaaaaaaaaacaaaaaaccgtggctcaggcctatgtggactcaggctgcgggtcccagcgttaagacagatgcttagcctggagggaggcacacaggcacactggaagctgggcttgggagtggcttggaagcagctttctcccgctgtcttgagctgcttttttgtttttgtttttgttttcagagaaaatcccatttagccatcagctgcactaacactagcagtagtttgggtctccccaaagaagggctggacctactctctccctttggggatgccccagtttctatttcttggtctccaagtcactgaacaaatagcctccttgtctcatcaacttttccctgtagcagatatccttgggaagtacccccatgctagtgtcccaggaagacccacctggagtagagaaagctcaggccaggcgtatcccactttcctctaaagtactccctgtcttgagttcttgttcatgtctttggccacgggagctgaagccaggagctggagttagtcgggcaggagcatgtgggtgatcctagcaacatttttgtttctctgtgttgattgtaagaatgtagtcaatgctgggaggacctgctgcagtctgcatcaggcctcctacagggaactctcagccccaaagggagagtacacaccagcttccatggcccattcctaccccacaccacatcctcgccctgaagctgctcaggcaggcgattgggggtactggactcggccaggctggaggtgagaaagtatacttccttactgtgtggccttggatatgtcacttcctctctgattacctcagaggagactgagaagagactgaggccatcagagaggaagtgacatgtccaaggccacgcagtaagggagtatacttctcctctcttgtttttattgactttcttagaattttttctttttatttgtagttgacacataataattgtacatatggaatacagagtgatatttcaatatgggtacacaatgtgcagtgatcaaatcagggcaattcgcttatgtattgccccaaacatttatcatttctttgtgttgtgaacattcaaaatcttctttcctagcttttggggaatagaaggtaaattatagttaaccagagtcaccctgcagtgttgcagaaaaccagaatccattcttcctgtccagctttaatttggtatttgttaaccaacctctccccatcttcccctccctgctacccatcccagcctctaaatacccatggttctactttcacggcctccgtttcttcagttgtaaaaccagagagattgaactgcagcttctaaatgggccagtcccctatgcgtcaccggccactctccagcagtgacgggcatagcctgcctgttacctggagagcagtaatcagcccagaaatcttgttgacaaagctaagggcagagtttccattggaaaaaagcagcttgcaggaaaaattgttgataagaagtggaaatctatctaagtaagccatatatctcctgctccactgcctgaggcctcatgggctccccactctgagtcctgccacgctccaagaagctgctggagttccccagagtagccttggtgaatgagagtatgtagcctggtttccatggagtgacccactagccctgtgatatggtttggctgcgtccccaccgaatctcaacttgaattgtatccctcagaattcccacgtgttgtgggagggacccagggggaggtaattgaatcatgggggccagtttttcccatgctattctcatgatagtgaataagccttatgagatctgatgggtttagcaggggtttccacttttgcttcttccgtatttttccgttgccactgccatgtaagaagtacctttcaccttccgccatgatactgaggcctgcccagccatgtggaattgtaattccagttaaacctctttctcttcccagtctcaggtatgtctttatcagcatcgtgaaaacagactaatacaccctgatactgcagggggtgtcagtccatggcttttagcaggttctcaaaggtgatgcatagtcccaaaagggttaatgaagaacaagcttcaggggtaaactccttcctacaactactgtgtcatcagccactgaatttcccatcgttcagtcagtcataactggtgagcctcttaaatgttcttagaggttcttagagctgcgttgctggggcccagcctcctctggaaagagccaagtggcagacagaggaggtggcagccctgctgggagcacacccagcactcacactgctccatgggccctttccatagaacagacactgttctctccaggtgcaggccacaaaggtggacgctgtctgtgctaccaccttactcactcttcttgtggggatcatgatcaaacctgggaatttgatggagtctaaatcaaaattaagtgtaacccaggctatcttctgctgcttctcccctgcaacttcactcacaacataagcattatagagtgttctatttttataattttctgactgatggaagtgaagtgtcttagggcttagtgtcttagaaaagtctaagagtcttagaaaaagggatccttttcctttttaatttttcttttcttctcctttgtttttttttttttttttttgagacggagttttactcttgtcgcccaggctggagtacaacggtgcgatctctgctcactgcaacctctgcctccctggttcaagagattctcccgcctcagcctcctgagtagctgggattacaggcacccgccaccaaagccggctaatttttgtattttttcttttttttagtagagacagggtttcgccatgttagccaggctagtctcgaactcctgacctcaagtgatccacccactttggcctcccgaagtgctgggattacaggcatgagccaccgcgcccggccagggctcctttttctaatgtgcatatggtaccatatggtggccccctaagccctcctctagagctcctgtgcgaatggcctagtagctttgtctaacatggctacaaagagctatctaggaggagccattgaaagctatggggtgctgttccatctgtgcatgatacagagctttctaagcatagagccatccaaagagagtggattgccgtgggaggtggttctccactaggaaaattattcaagcatgggctagaaaactgtttggctagaatatcatagaagaaatttaagatgattggactaggtggtgctgttgccttccaatcttgagattttcagagctgaatgttatctcttgggttttactgtggacattgtggtctgtagaaaataatttccaatcgttcttacacatatttaaaagatgtgtgtatgaaggagctggggagacagtcggggcaggaagtagtttggatatgttctatttatacagtaaaccctctccactccccattattatcactctgttctctgtcagcttcttctgggaggtactgagactgttctggacaaagaaaatggatacatggtggggatggtggagaagaaagagagggtatcaccttagctggccatgatattattatgcctgagttatgcaccagaataaagagaagacaaaaagttaggtaggtcatgcagtgcttagcactcgatggggcagagtttaatggatgatgaaacgaaaaatagcaagacaagggtgtgtacgcacaagtacatgtgcggttgtggctggcggacacacaggcatatcttctcctggaaaagcccaacacttggttgactctgtgtgtggcaattttaggaagaagaagaattgtctcccactctgtggggaaatagcttagctttccatttctttctagaaggagtaggaactttggaacaatgtcaacaattagaaagcccagtttagatcactccagtcccaacctggcagagagaggacccctctcctggggtcgattaggggccacatcttttgtgcccttcctgctcattggcatctgaggatgagtgagtctcactcactgaggcatgaatgttgtggttcatctggctaatgagggatcagggagaaagcttcatctcatttaaggtcttctttcctggcgtggctcacacctgtaaccccagctacttgagaagctgaggtggaagattgcttgagcccaggaattagaggctgcagtgagctgtgattgtgcaactgcactctagcctgggtaacagaggagaccttgtctcttaaaaagaaaaaaaaatagtgcttctcatgatgggaattccagtcttggagttgcatgctctcagagctagaaaagacattagcggtcagacttcttgtccactccatggtccttctcagagtcacagctttaccacccttaggagggtccagtcaatgtggagttaaactgagccatgagggactttgatttttgtcctcccagggctctgcccaggacacctggcaattgccaccctgcaagactgccgtaggcgagatggccccggggatgcccatttaacagaagctgccgcaagatggggccgcctgtggtgtgggctgacaggcagacgtgtaggagaggcaatggttgtggcttcagtgactggaaggaaacgtgttatcttctcatttcttgctaaggcaaggtcagcactggggtcagggtcaggcagggtttttgatgtgggatgcaggccctggagggatggaagccaggggctttctacagagttgagctcctccccctctgcttctctgaagcttagtgctgggggtttggtatttctaaagcagggtcaggagagggttgctctggggacggtcccagcaaaagcagtgggatgttctgtgtactcagaacatgaattgctggtggggtgggcgtgtgacgtggcagagggcaggtattaagcagtttttccacctgtcctcctttccaccttatgtctttgccccactttcctataggcttgcacttggacttagaggctgaatctaggatatctctcctaaaaggaccgtccactattggaaagtcgagtgaggaggagaggcccatctattgaatgggattttcccagatgagaggggctgggtgacagaaagtggatggaacctggctgaggcctgccatcccttggcctggggatccttggaaggaaaagaacaatccccaggttcttccctcatgacctggggattgttctctgcattgctcctgacttagtggaagtgaaaggtgtccacggcttagggttgcagaaatgactcagagctaagctacctagattcaaatccagctccaaagacaatcaccttccctgcgcctcagtttccacccctaagataggggcaataaagtacccactggaggaggctcttatgagagtgaagtgagtgaggacacaggaaaaccatggagcagggcccagtgcggagcagtaggcatctgctctgatgattgtcattgcaaaaggacccagttgggcactacaatcagcctgtcctcatttggccccaggaaccaccacttgcctagctgtgggaccctgggtaagtcactcagagtgctctgaaatttggctttgctacaagtaggactgctccctgcctcacagaactgttgtgagggctaaatgaaataatgtatgcagagcttagcaggcctggcatgtagtaaatactccggaaacattttttttaagttccagggtggttgtctatctggatgtcacctctgacctctgaaaaccacagggattgaggataggaaagcagtgctcctttctgcatccacccggtccccacctcaccttcctgagcaccaggaaaggagcctgaggaatcaataaggccagaggaggaaccctgcagagcgtggtcagctgggaaggacttgggcagtaggagcagagggggcaaaggagggcctgggttgggggtacgtggcagcatgcctgtcctcagcagacacctcccactgcccatgcttcttgtgggggtgggccagcccagcttaggttatcttggctcattgtccactagtgttttcctcagatgctccctgggagctggcagtactggagggggtggcaagtggcctcagtcggctcacagttctaggaccgggcccaggtcttggaagccccttgagctctcccccttccctgcttaggccactggaagacagaggtctccaaagaaagacaaaagctggggtctagacataccccatctggggtctgacttaaaggcctttgccagggtcacctcctgttggcatcagagaaggaaagaagtgtgtgtttgtgtgtttgtgtgtgtgtgtgtctgtctgtttgtctatgtctgcaggtggacaagtagggccgggtgtgagtggaagtggaaaggatactattctgcccatccctccttgctggccccccagccagctgctaagatccagagtctgggcagcagagtcaaccctactgcagctgggggtgttgagcatgtctggggaagagctaaaagtggcagaaaacatcctgtttgaaagcaatgctttgctgtatttaacccctgcaacacctgctccgcctacacccggtctccacagacaggagatctcagacacctgcctttgaagctgtcccaagaggccaaggctgtgggctgccatccaagcctgccccattcccagctcctgtgcggcacctcctctgccctgcctggggcagccgtcttcccgctcttagcagcaggacacatggcccagttgctctgcttcctgagctgcctacaatctggagatggagggggtagtgagagtgtgggtctccctaacgaaaaggcccttcctccctcctgacaccctgggctgtgagaggagaaggagtgcctaggcgggaggctgtttccttctgcctggggctggttgccgccaccgcttcccactgctcctgctactccctgcctcgagggagggccatcctggctgctgcccagccgccacccccacacccctgccagcgatgacatggcatgcctgctcccaacaagccacttctgtttgcagtcactgatctggggactaaagtccctggaaagagcctctcgtgcccacttccttagagactggggaggcggtcagcgctccgccttagataaaaggtttccccttcttcatttcagaagcctttgggtctgaagtgtctgtgagacctcacagaagagcacccctgggctccacttacctgccccctgctccttcaggtaggtgtttcctcatcagccgcaacttccctggctttctgttttcaaggggcggggtgggggggaggggcataagaaggtggtgggcaggggaaggaagggataccacccaggattttgcaaggtgggtcccgggccagcagagtctgcaactgagatgcatgagtgtgtggggtgcgggtgggagttcagagaagggctcaggagatggggcttgctggctccagccacgaccctggctgggctctcctgtgcgtctgatgtttcctatccagcccccatctcctctttctctttgctgccttctttagtctctgcctgtcattcctgggactttcagctctcaagccacagaggcttggacatctccacatgtggactctggtcctgggcgctggcttcttgatagcagcaaataacctcaagcagggttgggtcttctgtcagctcccctgaaatggtctcattcactgtgggcctctggctgcttgatccagcctttccagccttcacccccagcatagagacttcctgatgtcaaggcagcaccccaccccattgcaggactgccccgttgctgtgctgtggtagtatgttgttccactcgcttgcatcatagcatctccaaatgagtccatgtgatgctgaacatgtgttgactgatttaacagatattccttcctaccccccatttgatttctctgttttccacgaaatccaccggatacttgggagcctggatgacccagactctgtagcaacaccacgatgactcggagctgcagcatctcagctgacccagggttaagccacaagcatcctggacagtttcccctattaccccacaatgatattgggcctccgggatgctggccacatcttgaatgtgtgctatgttctaaaacctgcaggcatcagcttggacttgggatggtctttggggacaatgttgatagatccacaagcactttttctatttttaattgttttttaaattatgaaacgttttgtatattcagaagggcatgcaaacacagatatacaaaacatagatgtagtgttttgctaataataataagaagaagataccatatgctcactactcaaaaaatagaatactgctactatataatgcacacccctccccgatctcatctctccaaaggtttctgttaatcattcctttgttctttttttctgaacttttcttataggttcagggggtacctgtgcaggtttgttacaaaggtatattgcaagatactgaggttttgagtatgaatgaattgtctcccaggtagcgagcatagtactcaataggttgtttttcagcccctgcccctgtccacttgtattcccagtgtccattgttctcatctttatgtccatatgtacatgatgtttagttcccacttataagtgagaacatgtgctattttgttttctgtttctggctttgtttccttaggataatggtctccagctgtatccatgttgctgcaaaggaaaaaggacagtgtatgtgtgtaaaaaggacagtatacgtgtgtaaaaaggacagtatatgtgtgtgtatatatatatatatacacacacacacacatatactgtattttctttatctagtccagagttgatgggcacctgggttgattctgtgtctttgctatcgctgcaatgatctttgttctctttctgaaagtgcttctctctttatatataggtatatatttatacttgctaaactttaatgtatatgtagctgctaaaatttaatatatacattaaatatgtatttatatatttaatatatattaatatataatatatattaatatgtatttatatatttaatgtatatattatatatacattagagtttagcaagtataaatctagctgtgaaagaaattagcaatagtgtcactattactattaggatagttcaaaagtaattgcgatctttgccattatttttgatggccaaaaccacaattacttttgcaccaacctaatacattaaggtttccaggaaaagaaaagctaaatgaggttagggaatctccgaggtctgtgaccgggattccctctgtccctttgggactgatgataacatattcttgcttatctgcaccacttctttcccttggtgtgaagctctttggggaatttttagaaagtatttgttttattcatttggcagtagtggtttctagacatatcttaaggtttgggcctctctgggcctcatttgtaaaggggatgatgataatagcatctacaccatgaagtggtatgaaggtgaaataagacttaatgagctttgatattccacaccctagatcagagatcatgggcctagtcattgaaaagtagctcagagcctcccaagggcccccagaatctgcctctgtcacccaagggcaggaggaaatggtaccctggggtggagtgggttcttgtctcttgtttcctggctttctctcttatttttcttctgacaagaaggaccctttgcctagggtcaaaggggtcactgaaacctgtaatgacccttttgaggattcagataaaattgggagaactgggaggcagtagggtctgaaagcatttcagggcagtctgaggtatcccagaatcatctctgagcctgacagtagacgggatcagacgcagcagacaaagctgggggcccagttttggctaatgaaagagtcaagccagctgcttcctgagaaggccttcccaaagctgtgggctttcgttccgtctgtctcttctccttttcctcaagtatgaaatccatctctagatgataatgcctgtttagaaaaaccatctctgaaaacacaattaattgtataggactcacatgactcagaaggacattcaaaataatgttttaagtgttattgccaaaaaaaggggggggaaatatcttgaaatgttgattgtcttggtacaggaacaccaggggcataagcctattagccctgagctttatggttgtgaggagctggggctggaatgaccagggcacctaaatcctcaattccccccaccctcaagaggaggagacctgagggtttctctccacatgtaggtgctgaggctgagggaggactctcattttcccttggagggggcgttgggcaggatagaagcccctgacctggttcaggtctgtgcctgaggcagagctagtgccagtagcatgaatgggttcatgcatatgatccttacaccctggaagtaaaacacctcttccaatgcagacagcgggggcatgcagaggtgaaccactaaacccaaattaacctgacagatgcaacatctgaaaccaggcagctgattccaagccatgctctgagccagctatgtagggcgaatcatgtatgagggctccgaaggcactgtgctcaggcctgggccctggggagatgcccacccttgctgagctccctggtggtggggggtgggggcggtgggatgaggctgggggtgggtggcaccaaggatgccagctggccctggcactgactctggctctgaccgtggcctgcttgctgttcttacagggatggaggcaatggcggccagcacttccctgcctgaccctggagactttgaccggaacgtgccccggatctgtggggtgtgtggagaccgagccactggctttcacttcaatgctatgacctgtgaaggctgcaaaggcttcttcaggtgagccctcctcccaggctctccccagtggaaagggagggagaagaagcaaggtgtttccatgaagggagcccttgcatttttcacatctccttccttacaatgtccatggaacatgcggcgctcacagccacaggagcaggagggtcttggtgagtggtatcttcttttccctcctctcagctccagatgttcctctgactctcttggaaatcgctttcctgaggttgctgtgtgggtctctgtctttccattacgcctgtaacccacagcctcctacaccaacccacgtgtccatccttccagagtgaacctcctccctgttgatgatcacagcttcctcacccaagagacaggcatgtctttggggaaagcccaagaacttggtttcagagcttgccttcccatccaatccaaactgttccttggaacaagggaaatggcacctcttgtcgggtcatcacgatctgtacccatatcttcacccaaggactgtttgtcctggtctgaaagccaaccttggaacatccaggcagtgtcaggaatgtacctgcattcctgtttgatcagggccagtttctttaccaacacactccccttacatgagcccaggattacagatgtgaaaggtgtgggaaaagcactggaggttcccattcaaagccagggtgggagcgtgggaaagggatgaattggggcaggaactgggaatcatgagaaattagcatttggcatgtattggagagagagagagagaatagcctgaaagaaggcagccaaaacagatcttctgtctagcggtctagactggaggtggctatggcagggctctaaccatcaaatgaggaaagcacaaatcaagtccagaggaggatgctgaggtcggcttggttgttgtctaaaccggagtgctctcctcgccttgggggcacagtgaattcaagtccaggcgcttgtgtgggactcttactcaaggacttggggtctctctgtcaacacaagctcctgattcacctgccctctgcctcaggaatcagcaggcccagagtttcatggccttgagcaattgctgggcagtggggtttctgtgggtgctaattgcctgtttggcctggcactggctgcccgcttggcttcccggcagcctactctccagctcggggaaccagacaagcagcatcgctggctctaagtcgtgttgctgcatttgccaatccttgggcctgaggtccacacatcctgcagggtgggccttctagagccccagttgtgtgtcccaggtgacacatggaccctttcctgccaggtcctctaacttggggggctgccttgagtgctaatgagaggggaatctaacgcacacctcagcgcctgcttactaccatgaaacccatcagaaaggcatggtctggggtgctggccatggcaataatttatgggatgtccttgctcaaatggatgtccttgacatatctaggttttagttaactcaactaatggcatgcatgtattgatatccaccccctctgctacatagtgttaatctgaggattaatgagatgacatgtaaaaaagtgctttgaaaaacactttttcagtctgatgaaaaaagctgagatttttgagcctgatgggtcaccactgctgcccttcatggaaccatgctctcataaaataaacaaaagcctcgcagccagccagccagccactttcctcgtgtgtgtgtgtgtttgtgtgatttttttgtagtgatggggcctccttatgttgcccaggctggtctcaaactcctgggctcaagcgatccttccatcttggcctcccaaagttctaggattataggcatgagccaccatgtctggccttgtgtttctttcactcattccgtcaccagacttcaatctgcatttataatctggcattgggctaggagttgtcaatatggagattctcaccgaaggtcatatcttgtcagtctgcaaccaaagcatttggttatggagtctctacccccaaatccactctctcctcctaggcctcctcccccctgagattcagctctgggaaatgagaatcttaggtggcagctggttgggtggtgacacattggaggccagttcctcactggagtggctctgactgctatgcatctgtagttgctgcccttggacacaccactaggctgggaatcctcaggacaggagcatgtgaggcatctgggtggagagaggacaggtcctgtcatgccccaggctgagtgtgaaagatggcagaatgaacaaggatggtatgtttgtaatctgtgtcaccacagactgacagagtggctgtgttgcttgtgggcacatgatgccaccttaacccactcttagtccaccttgacaagagcccttagagtctgttgctggctgttggtcacaaccactgcctgcaatgcctggcactatgggctgcaggctggttttgtcttgttaccctgtcctcagtctaccttacttagatctttactgtctctgtcttgatgactaagctaggctgctacattctaaagagccaacatgtctgtcatttgcttgaggatgtggatgaaagagaatgagtggggttatctatggattgttcaagagtaatgttcagaaacttgagggaaggtcactgaagctgtcaagaaagacagctgcaaggttctgaattttgtttgatatgtacataaacaaacacacacatgcacacacacacacacacacagtcaaccttcattattcatggattctgtatttgcaaatctgcccacttgctaaaatttaccaaaatcaatacttgcagcccctttgtggtcatttgtgaacatgtgcagagcagtgaaaaattcacatgacttggcacctatcttcccagccagggtcttcacaatctatttagtgctacattttttgcctttttttgatttttattggtgactttgctgtttaaaacagttcccaagcgtagtgctgcactgctgtctggtgttcctaagtgcaaggccgtgatgtgcctcacagggaaactatgtgtgttagacaagcttcctgaggacaacagtgctgctggctgtttgatcaatgttaataactcaaccaacaatatctattgaataagatatctttaaacagaaaactcacataagacaaggttatgtgttgatcagttgatgaaaattttgtgaccagaggcttgcagaaacctcaccctgtgtttcctccaggaacagtgtttcaatattcactaatccagtgtccacagtgactatagaccataactaccatgaataatgagaatcagctatacatacatcatttctcctcttcttccacccctgatgcctgcttctccttctttgcttcatccaaattttatttggaagttttccattttgatctggtccaaatagttgcttgagaaccctgtggtcactcatatctgtttgtgaaactctgatcccaggaagcaaggacaatgtcagtggtctgtaccttctctgtggtgggtactgcatccttgcatccttgggaacacagagatgacaggaaccaagtccttgctctcaagaagcttgcttgaccatttcctgatagttattgacagacagcattgcttgaatattgggtcactagctcttttccaagccctggagaccagtaatccaatcccatttgaccatttagtatttggtttggcttctaagatagttaactaaactgctctaggagctagttgttatcatcaaaacgagtctaagactcataatctagctgaagtgtgatgatggttagaaggttagagagggatcacagttctattgatctatgatcaggcattagaggccattgctggtcaattcctcctgcaagctatttcatgttgcttgtgcttcctgttattctggaatacagggacatcctcagagaaagatgatatttccagtgtgaatataaggttggcacaggcaggcttatagatggccagacacctcttggctatatgttaacaactaaagcataagtaagagccagaggaggaaaaacatttggaataggtctattccaaatgacatatatagtggatgatccatatatgtatatgcatgtggatgcatatggtcatggatggcttcgtccggagtctgatataaaggaaaaggtgtaatggacagagaagaaaatcagaggaacccctttgatgaagagaatgaaggtggatggtgaggtttaagagctgatcctggaaggcaagatgagaaacaggtcatcgtttgcctgcttatcttgtcttcttcctccctgttgggatgcttaaataaggactctgtgcagctacaagctaacaaagacagtgcagagaagtgcgttttcgcttcctagctccaaggttcttgaggactttgttaattatgggtcatgcggagtgcagggggcaaaaggtaggctggcgaggatccaggaagatgaggaatgttctggcattcaggaaggtcaccccactgatatttgtagctcttctagcaacctgatgtgaaaggaagcagagaaatagggcagatgtccaggaatttaaaacctaaactgcttaaaggagagaaaatagagaaaaaagggaggaacagccacacagggtattctatgggcacaagtaaatgagtgaccaagaagtcagtgttgctggagagactttgtccaggtccactttggcagctgacctccattcacagatattcaaggatgtgaatgaaagagaatgagtggggttatctatggatgttccaagagtaatgttcagaagcttgggtagaggaggccaaaatatttggagagggaaggtcactgaagctatcaagaaagacagctgcaaggataggattttacattacctttttgtcattcttttatttcttttgaaattcagcactctaatcagggctcatttgcatgactttgcactcagcacacacttgagatcttccctgtgcttgggttatacagggccagtggagagcatggtcagatgtgaccccacacttccaaagcatccttctagagactgcctgaatccctagagggatttgtcctagaggagtccttcaaacagcctctgcttcatgctcctggactttgggaaagcatgtttttgactgctgctctagcttggattgagagatggtacattcctgatgagaaccatagtatatatgaagatcagtgtattagtccatattcacactgctataaagaactacccaagactgagtaatttataaagaaaacaggtggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgaggtggcgggcgcctgtagtcccagctactcgggaggctgaggcaggagaatggcgtgaaccccagggggcggaggctgcagtgagccgagattgcgccactgcactccagcgtgggcgacagcgagactccgtctcaaaaaaaaaaaaaaaaaaaaaaaaaaaaagaaaacaggtttaattgactcatggttctgcatggatggggaggcctcagaaacttacaatcatggcggaaggtaaaggggaagcaaggcctgtcttacatggcagcaggagagacagagagcaagtgaagggggaagcgccacactttaaaaacatcagatcttgtgagaactcactcagtatcacaagaacagcaagggggaaatctgtccccatgatccaatcatgtcccaccaggcccctccttcgacacatggggattacaattcgagatgggatttgggtggggacacagagccaaaccatatcagtcagattccttggagtcaaacagttcttgattctaattccagctttcagacttgctagctgtgacttaaagcaagttatttaactttcccgtgcctttttgtgtcacttgtaaaacagggataatatctacccaaaggttgtcgagagcattggagatagtatgtaaaatactgacctagaaagcttccagtggtgatagctagtatcattatccctttttagtgtcttagttttgaggacagatggtcctttcttccttttctctaccatggaacttggaaagtataactatgtgatgtgttggcagtggtctctgaaaagaggttcctaaacagaaggagttaaatatcaggtatgaagagggaagggctgggccaggggctctgagagagcttcatgtcggtcaaaggctgggtagaactggctggtgctcaacagaactggacagtggttgctgtaactagcacaggggctgtggctctagacatcaggagctacagcacatgaaacagaaatatggtttcaaactctgctgcctgcaggctcccatgctaggcacccagagagcaggcctaagacatggtgtctgcttcaggggtctcaaattcttaatgagatgtttaaaatctactttaaaatctactttcacccactctcagcactccctcccactgcctctttctgctagtttctcttctttccctttatttagggtttcctttgtccaggtcctgttcccttttcctttatttagttcttacaaccctctctgaaatgttgctcccattttacagatgtggaaactaatggatgggaaggttaagtaacttgcccaaggttgtgctttaagatttaaactcaaacatatcgatctaaccaaagactgcatttcatttttaatgtttaggtagttgtagtgggtagtggattttttaaatgtaacgtcataatatggctttttaaaaagccaacagtttaagaggatatgtaagtgaaaagtaaatcacctattcaacccaattcttagttccctacctcctccaggaagctgtcactgttgccagctcatcgtgttcgcttccagattctttatgtaaaagtgcatatgtgtgtgtgtgtatgtgtgtgtgcacacacgtcaccattctgcatcttggttttatctgctaaagaacacttcttcaagctcattcccatttcagcattcttcctctttctttttcatagtcacagagtattatatggaggttctgtgagataagaaaccagtgcctggctgggcacggtggctcatgcctgtaatcccagcactttgggaggccaaggtgggtggatcatttgaggtcaggagttcgagaccagtctggccaacatggtgaaaccccatctctattgaaaatacaaaaaattggccaggcgtggtggcacatgcctgtaattccagctactcgggaggctgaagcaagagaatcgattgaacctgggagccagaggttgcagtgagcccagatcgtgctgctgcactccagcgtgggtgacagagtgaaattccatccagaaaaaaaaaaaaaagaaagaaagagagaaaagaaggaaggaaggaaggaaagacagatagacagacagatagaaagagagaaagagaggaaggaaggaaggaagagagagagagagagaaaggaaagaaagaaagaaagaaagaaagaaagaaagaaagaaagaaagaaagaaagaaagaaagaaagagaaagagaaagaaagagaaaagaaagaaaccagtcctctgtcatggtcatttaggttcggtctttggcttctccagacagagctgcagtaaccaccattgcgcccatggaccgatctacccacaggataaatacctggaagtggctttactgcgttaaaatgtctgtgtgtttaacatgcttcgcattgccaattgccctccaaaaaaaaaagtctgtgctcttttctacagtgctaaccatcctttaatgtttttttaaacccacctgaggagaaccccctgatgctgcctctcacatacatgtaggcccctacatcatttgatgtaggtctttttattcctttagatttgctggggtataattgacagatagacatggtctatatttcaggtgcacaactcgatgttctgctatacatatacattgtgaaatgatcaccataatcaaactagtaagcattcccagcacctcacatagctttcagatcaggagctctcgctagttcctgtatcctgagcagacgctggaatctctgtgacagtgcagtggagatggagcccagaggggatagttgaccctacgcctgggttatgcaacgtgcgtctctgctggcagaggccacctactggagaaaggcccaactgtcccaggcctgaggccctggccccaggctcttgatgcttttgtgaggtttttgtctctttctgttttgataaactggtctctggcatgagaatcggtcaatgtcctctctcacccctggctttctagaaactgcatctatatttagcttggttgccccacccctacccccccttcctgagctggggtataaatgccaaccaaccagaggatgacagggtccaggctcagagagcagctgaggcaatgggctctcatggaaacctgaagctcttgtttctcaaatccaaaccagctcacaggcaattagtattgggaggaaggcagggtagggtgtagaccttcaggacaaagcacagagccagggttgggcagtctggctgccctgactcctcgtgggcagagagtaaatgacagccacacatgtggaagtgcccttggaaggcaggagaacagggaagaacaggacctctgagccaagaggatctgtggcccagcaaacagacatgttgggccagacacacctgaaaggccagctctgggatctgagttccagagagcctctgggtctggcagttggagctggggagcaaactttctataccctgaacactgaccccacgctccagagcgtaatggtgtcctcttccttttcagtgttctcgggcttcatatgacaactcttaagcagaagcaagggcgccaaactttttttttacccccagtactttctcttttattttttatttctagagacaggatctcactttgtcacccacactgaagtgcagtggcacaatcttggttcactgcagccttgacctcaccagctcaagcgatccttccaccttagcctcccaagtagctgagaccacaggcgcatgccaccatgcctggctaattttttttaatcttttgtagatacagggtttcaccatgttggccaggttggtctcaaactcctgagcctaagctatctgcccacctcagcctcccaaagtgctgggcttacaggcgtgctcacgccactgcacccagtcccagtactttctcttaattcagctctgcactattttctcttcctattccttttttttttttttttttttttttgagatggagtctcgctctgtcccccaggctgaagtgtagtggcacgatctcagctcactgcaagctccacctcccgggttcacgccattctcctgcctcagcctcccgagtagctgggactacaggcgcccgccaacacgcccggctaatgttttgcatttttagtagagatggggtttcaccgtgttagccacaatggtctcgatctcctgacctcgcgatccgcctgtctcggcctcccaaagtgctgggattacaggtgtgagccaccgcgcccggccttctcttcctattcctagcctcattcctgttgtcaggcaaagtggggctgagtggcaatctccaaccctcctgcgtatagacatctgagatggagcttcatatttaaagtgacatgagaaaaatgagagaaagatggcgaagcagtggaatctcttttcaggcaaccctgcagctgggggggctgcccccaagtgagggtcaaaggcaggctccctggagcctggggaaggacagacggggcctctgataggccctggggcctcaagaagctctcagtcccgggcccagtctggtgagaggctttggctcacatcactgtaggtggtggctgggctaggctgacgatgtgctgtcttcttggtgcccatggccttgcaggcttaacaggaagagctctgagccagacaagacagccagtgggaggacagagcagcccctcagtgaccagagcgaaatgcccggttgttgaaaaacaaaaaaaaaaaaaaggaaatgagagtttcttctgaaatagaaacttctggtccttgagtaagtttagagaattacgggcattctgaggcctgagcatttgtggtgacggatgaagcctcaagaaccacaaggttggtgggagggacaccaatctcatgtcctggaacatacagatgtccctgtggggataattgtatctcgtttctggggaaccctaacagttcccaagatgcttccatattctcttgtccctccagaaaagcagcagtaaacaaatagaggtgaacggcaaaaggctttttgtttctacgaagatggaaaaaagcctggcgtataacttctttcttgttagctactgcagggttaggactgggcctgaggcgggctagacttggagctaaggagcccctgatagcctggtgctgctccacctcctgacaaccctggctctgcagtaggccccttgggtgatgagggttgtcacagcagggtaccagagccaaggtccaaaaccaacagcagctgcttccttgactgttgggtcattcttggcattgagccacctggggctgtttggggcatcaacttcactgagcactttaagtttctggggttgaaaacaatccaggaagctaaaggctaagccttagatccctaagacttccagacctaggagcctgcacttcttgctgaatatcctcacctgtaagtttcttaacctcagtggtcccacgtataaagggagggagttacactgacggtctcttgggccctctgtggatctaagagtctgggcctgcctgggactgccagtagagccctactctggtctcttctctatcccaggggctgagtcggtgtggtccccagctgtccatttgctagagcaagcttgacaattgatgagtgcgattcccctcaaccccatgtatgttctagtgaatgtgaacagtgagtcatgttttaccaagaatcctaactaatgcctggcccctgagcagatgacgtcagtagctcatctccaggaaggaaatggttgggcctgggctttggcttggaaggcttgggcatcttcacactcagcagttccttggaagatgctgctgctcatgcagacagtgattctgccaccatctttccccatctaactatgtcagaaaagtggggcctactcctgctggggctgggaggaggacaggactctcaggacatggatgatgaaaagcctctagggaggtgcctcagggaggtgtcctttatgcagcctcccaaagtccacgtggtgtggctggcagtgggagagaatgttcgaattaggaaaatgagcccttaaatgtgcacacttgtgcacacacacacacacacacacaacttacataggctacaagggtgccacttttctttttcttttctttcttttttttttgagacagagtctcattctgttgcctaggctagaatgcagtggcacaatctcggctcagtgaaacggccgtctcccaagttcaagtgattctcctgcctcagcctcccgagtagcggggactataggcatgtgccaccgtgcccggctaatttttgtatttttagtagagatggggtttcactatgttggtcaggctggtctcaaactcctgacctcatgatccacccacctcggcctctcaaagtgctgggattacaggcctgagccaccacacccagcctcaagggtgccacctttctagctaagaacacttcagtagttttctgggttttttttgttttgttttgttttgttttttgagacagggtcttgctctgttgcccaggctggagtgcagtggcatgatcttggcctactgcaacctctacctcctgggttcaaacgactctcctgcctcagctcccagcccccaagtagctgggactacaggcatgcaccatcatggccaactaatttttgtatttttagtagagacggagttttggcatgttggccaggctggtctcaaactccttacctcagatgatccgcccacctcagcctctcaaagtgctaggattacaggcctgagccactgtgcccagctctagttttctgttcctacagagctcctgcttcctcttcctttcaaaaaacccaaggccaggcctcaggatttccacctgcttgtctggccccttctttttctgggcaggttctgggatgtctagagctatggtttgggccttttcttccttccatgtacacatctatccctggaacaggagctattccagtcacaggtctctagaatctagaagacttcatgctgagactagcatccttacttctcatagcggctcattaaatgttattatgctggctactctggagatttcaatatttaaaaaggtttcttcggccaggcacagtggcttacgcctgtaatcccagcactttgggaggccgaggcaggcggatcatgaggtcaggagatcgagaccacagtgaaaccccgtctctactgaaaatacaaagaattagccgggtgcggtggtgggcgcctgtagtcccagctactcgggaggctgaggcaggagaacggcatgaacccaggaggtggagcttgcagtgagctgagatcgcaacactgcactccagcctgggcgacagagcgagactccatctcaaaaaaaaaagggttttttctagggaaatgcacttttgttatttcctgtttaattttttaaaatgggaaggggaacagagtactgtaaaataagtataagagtcggggcgtggctgtgcgcgatggctcacgcctgtaatcccagcactttgggaggccaaggcaggcggatcatgaggtcaggagatcgagaccatcctggctaacacggtgaaaccccatttctactaaaaatacaaaaaaaaattagccaggagtggtggcgggcgcctgtagtcccagctactctggaggctgaggcaggagaatggtgtgaacccgggaggtggagcttgcagtgagctgagtgagccactgcactccagcctgggtgacagagcaaaactccgtctcaaaaaaaaaaaaaaaaaagagtcggagtgcagtggctcacacctgtaatcccagcactgtgggaggcctaggatagaggattgcttcagcccaggagttccagactagcctgggcaacatagtgagaccccatttttacaaaaaaatcaaaaaattagccaggcatggtggtatgcacctgtaatcccagctatactggaggctgaagcaggaggattacttgaacccaggaggtccagcctgcagtgagctgagatcatgccactgcattccagcctgggctacaaagcaacaccctgtccccccaaaagaaacaaaaattaaaagaaaaaaggtaagtacaagccatgattggagctgggcaggcaatgaaaggagaagtaggaatcgtttggtgcccagcctagaggtgagagtgactggcagctggggtgggcctcatgtcttctgttggagaaatggagaccagggggcccagaagacaggtctccgtgatgacagggtgaggagccggaagttcagtgacccagggcagggtgtgtgctctctcggcaggcgaagcatgaagcggaaggcactattcacctgccccttcaacggggactgccgcatcaccaaggacaaccgacgccactgccaggcctgccggctcaaacgctgtgtggacatcggcatgatgaaggagtgtgagtgtccaggggctgggcagggtttgggcctgaagtggagtcagggaaaggccttggccactctcctgcaagtttgggcagagggtctgcctgcccttcctctgtagctgccagcatctggggccagggcctcagtgggaccagcagctggtgacagggcagctggaagtccagggtcagatgcactcagcggccctgtgcacctcttgaggatctgtgtgttggtgtcagaggccctggaagggtccctccagagtggggcctgagaggaaggagaggccggacactgccttcaagagtcccttctactcctgggtcagggtcttcctccaggatgtcattcttttttcacagctccctgttactcggacctagagggaagaatgaggttcaaggacccccaggttctatgggcttgggaagagagggctgatgtgggttaggaagggcaggagtgatggggagaattagtattcagagcatagttggcatccacgttctgtcccaccccagcctcccagcctctctggcgccttgagcagatctgagggcttgtgccagggagagaccaggaggaaagagtctgccaggggaagcactgggttctaggacgaccctctgaatccagatggagaaagaggagtgattctataggacttcctgtccctctctggggttggagaagaccaacatggcatatttacatggatattttgacccatcactgaaaacaacacttgaactttgcatcagagctctaggacagttatttggtaactagagtaggcattgaattcagtagatgctgggaggggccagcctggccctctctgggctggagcaaggccagctgggcatgggtgctctctgtacactcattcctttttctccttctcttgctcactcctgtctgccatctgcatccagacccccacccggccctaggacagaacccaggccctcctagctgtgggtctgaggaatcggagtcggagtcggggtggggatgttgctcagatgcggaccctcctggctatgggaccgtttggagtggttggggatggggagaggtcaggtaacaggaagatgtgtcagggacagaggataagtcacagaacagggcttagaggatagcaaatttctccgttaatgggaaaaaaattatctgttgttgggacacagaggcagagctgaggccctgaccctgggcttcctctttgggccttgacctaggcttctcttctgtgggtcatgactcctccctcctgatctgacggctccccagccaacactggcagccctgaaaggtgtttccagggctgtggtttctccacaccatcacagggtgcaggcctgggcacgctggtcgctcctaccctagtccctgccacgccctggctcctgtgtttatcctggagagaataagaagtggaggctggaggcccgggtgccttaagaggcttcacacacattctcagtgggccctgctcagggtgaggcgttagggtgggcaccaacaaggtgtgctcagcacagtcccatctccgcagagaagacagcctctgcaaagcagggagtccggtttctaaagctccagctaaccaagactggcacgaggttccactgcagtggttcgtaaggcactgccacaggagttcccctcaggactaagctcactgatgcccaagaggcccctctcctacctcaggaggaagaggatgtcttactgacttaaaatagaaagaacatctgagactcagagaggtaaagacctcaggtctggggtcagaaagcaagttggtggccaagctgggactagaatcagacttcatgtcccctcctacctgcctcctggtccccataaacagcgctgcatccatggtgaagagcagcaccagcctggggtaaatcagggggccctgcccaggagcaccctaccacgtggtgggaacccagcagcccagaagcgatgtccaccccatccctcagccacgcccaccccagcctaattctctcctgtggagtcctgtgtccccatcctgctgtatgcccaaggtagcttctcgccacaccaccctcctcatcccagtgcagggagcagtactcagttctagatgggctggtggagcggggatccagttaaaatagaaacgtcctgatgctttttactttcctgaagggaagactgtccaggaagagacattcccagcctcaggttagtccagcttcaggaggcctcaccagtgtgaagtcccccggcctcagaaccctgggagagctgcacatttcttatctgggctgggttttgtccccaaggcatagcatcccagagacaattgagtgtctcaatatttgtaaaaccacaggaagaaagctaaaagcccaggctcctgctgtccgagcaaggaggtgggccttccatagaagaggcacaggaagggaaaggatgaggacagaaaccctgtgtattgaccaactactgtgtgtcagatagcacatcaagcacatgcattttcttctgaaattctcacaacactccctaaatacgtaaatacttttattttttcaatagctgaggaagctcagaggaattaaaatatcatggctctcagctaataagatgatggtatcagcattcattctaatctaggtctttctgcttccaaagggcaggcttgtgggccacacccgaggcagcctctcgtggccccagtgggtcggagctcactccattgtgcatttccaggcactttcacatgctctaagagatggattgaagagagcttggtcccaccaaagactcattttctctcttttccattcttagttgactttataccctgggaacccaagaaattttataactgagttcttgctttttgcttatactattacctgtcctgcacagaaccacacattgtggtaacttgtttgatgtttttacagatgtatgtcttttctccctggtgttagtaaagtacctggcacatagtaggtgctcaataaatgtgtggaatcaatgaatattagctcctcattatgcttctttctctctgtatatcttccacaggtctatagatcagtaagattctcccaaacctgatcatgtctgtgccgttcatttggaaacattttatgtcctcttcctgtggttgttcttagcccatccttggcatcttgaaatgttttcaaattgtttatgttgcagatcttggcttcgttaaggagagaacatgtcttgcatgggaataacttgcgcaaaattatttcacactcagcaaggagcttaaaatgaagtcaaaaaaagcttctgagcagccatgtaggttttacaaagtccacatgccaaaactcatgcactttagacgcctgatcaccagacagcccaacactctttcagaacctgtttactcttattctaggtcaatggcttcatatatcatatagtgtcttcctatatgatagtaatgacatcttaggttcaatccattgaaaaaatgataagaaatttcccatgaaattaacaagatctttaaacaaattatttcgtaaatcacagtgcatttgcatatgtgaaagactttagacttattcagtcctcaagcaatgttgccttgcagaaggctcatggattgggcctgtgtgaaactggtagatctcagcatttcttcctctgttacctccatagaagatggaggttgctatttgatgcaagtgactgggaggaatcatgttatagggttaaacttgaactttctttgtctctttaaagtgggtaatttacaagctttgtgacttaattttattttcacactcttcagatggattggaacacaatgcctgtcaaaactccatggctgaaagccaaagtccgcttataaccagatgtaatcagacacagtagaggctagtggttatgaccttccactccagaaccagactgcccaggtctaaatgctggtttcaccactgttagctgtgtgactttgagaaaggtagaaagcctctctgggcctcagctccctcatctgctaaatgggaataacaacagcacctgccttaaagggttgtcatgagggctaaatatatgagttaatatacaaaaggctctcagaatagtgccttatagatagaaaaactctttatgtgccatccagcattacgaatattttctttttattacatcaaacttgatcaccagaacttctagctcccaagagatcagaagtaagtcttaagggggagaaaggcacacatccagaggcagacaccaataagaagacaacgcatagtttaacagggaggtggacactggaagcaagaaaagcagcccaagaactccaaagcccagcacgccaagccatgcaatgcggggcagacagcctctgacaactctgaggctgtaaccttgtcctgcaatgttcagtaattattcagaatgatacctctgaatcatcagggaaaggttatatgacgttaaaagtgttccgttacaaggttttctgtcttgaaaatctttccataacaattgtttcaataaaagaggtcagctttctcagctctctggtgtgccaggtgccattcactacattgcaggagacaagcagcactagagtactcactagcctttcctgaaccaggaaaatgatttgcacacagttggtgtaatctgtgtggatgcatttgatatttggtgtcagactattgagcagacaccacggccaggtagcccctccggtctagcctttatgggggaaatataagaattgtaagacaaaggccgggcatggtagctcacgcctgtaatcccagcactttgggaggccaaggcgggcagatcacctgaggtcaggagtttgagaccagcctggccaacatggtgaaaccccatctctactaaaaatacaaaaaaattggccaggcatggtggcatgtgcctataagcccaggtactctgtagcctgaggcaggagaatcgcttagaacccgggaggtggaggttgcagtgagccgaggtggtgccactgcactccagcctggataatagagcgagagtctgtgaaagaaagaaagaaagaaagaaagaaagaaagaaagaaagaaagaaagaaagaaagaaaaagaaagaaggaaggaaggaaggaaggaaggaaggaaggaaggaaggaaggaaggaaagaaggaaggaaggaaggaaggaagggaaaggaagggagagaaaagaaagaattgtaagacatggaccctgcccttaagtaacttgtaatctagagaaagagaccttgaacttcttgggctccgtctataggtaatgaattgaaaactgtgctaaccatgagtcttacagagcagaagataattgggtgtcaatgtgtgtgggaaagactaaatatgtagcaggcataggaaatgaggctcagcagaaaaaaacaaggcttgatgcagatcagggcaatcaagaaatgcttcatggaaaaagatggctatgatgtaggcactgaagaactggtagaactcatacaggtttgggggagagaaaacccagctggacgggcacctagcacaactggaaatgcaggggcgagcatgagcaggtcatgttcccaggccagcagcaatgccagcatgcccagaacagaggctgtgtccagaagcactaagacatgaagtctgaaagttaggaagaggccaactttagtttggacgtggtcatcagtaggggccgagaaaagtatctgggcaggagaatggcatcacagaatcactggaaagttagcaaagtccagtcaggctgagctacgtgcctgtagacaccatggtgtggctggcagaaacaggtctccctaactctggtccccaggtgagcaggaaagacaagacacctaatcttgggctccccaagcgagggcttacccccattgccttccctggatgctccctgccccttcctctcccaccctgcctgacctggtagggtctgtgcagacacccactgtgggaggagagttggcagctgtttgggcaggtgagtcagtcacctgggtgtggtctactcccaggctccctgtggagggaagcaccatctcttgcagtagcgctcgctttcctgccatgcctgtggcattggctgtccactccctcccagcataggctcttcctcaccccacagcaacttctctcgtcctccccctacctgagggcctgggccaatggacctttctcaaagcctggaactcacccaccctgggcttctggttcctttctctgctcttgtgaaactccccagttctgtgggcagatgccctggcagcagcagcaccaagcaagtagctacaggccaaaggccctggtgcccactcctgccgtcagcactcagcaagccacagccaggaggtctgtttgcccagccgccgtgccaggcacctttgctcccagcatcccctccacccccacagctgccccagcacagggaggcaggcagaagcctgcaggtggtggtggggctctctatggccccacaccctgattagtggctggagagaacttctaagattagagctgcaaggcctcaccctttggggccttcaagaggacttgaaaacttccatggacagaaatgtcaaggctgtacatgctggaggaggggtggttttgcagagaaccaaagaggtctagtctttctatattccacagtcccatgtctggaaacctggatgtggagacagccacatagaggagtcctctccacatctcacttccagcgctatgagagctctggcatcctctcttcaccctgatatcttcttcaggatcatggaacatcagggtagggaagagaagagaactaacatttagtgagcatctgctctgggccaggcatgcatccagttctcaaatcagttgatcctttgaggcaggtattattacttcccccacctgcatgtcaacacacacagacacacacacacacactttatggaggaaggtacaagaaaggaaccttggaagaacttacatggtccaaatctctcattttacaaacaaaagaacaaaagcccagagagtttgcccaaggtcccacaacttgttcttgagaaccccgactccctaaagcccagatcatcccccctctcaaatcctctttttcttcttcttgcatgtgccttccttttcaccatagcaaacccaatttttcttcacacagtggagtgggagtctccgtggcaggaggacccccgagggagccccgagtgttaaagcccctcctatcttggacctttacccccaaccgcaggaggaaggtttcctggaggagctgctggccagccctcctgactccccttcccaccccacagtcattctgacagatgaggaagtgcagaggaagcgggagatgatcctgaagcggaaggaggaggaggccttgaaggacagtctgcggcccaagctgtctgaggagcagcagcgcatcattgccatactgctggacgcccaccataagacctacgaccccacctactccgacttctgccagttccgggtatgtctgcctgctgggaggatgagccggtccagaggagaagcactagtggagccagggcccagggagtagggacagagggcaggggacatcctgaacagaactggggtagggacggaggctgctctgcccctggcactgggaggcttcgccttcctgtagaccttcctcaaagccattcctatcagagatcagggccaaggtaggaaggcaaccccaaaatgtgggtctgagaccccatctttcccttccagcctccagttcgtgtgaatgatggtggagggagccatccttccaggcccaactccagacacactcccagcttctctggggactcctcctcctcctgctcagatcactgtatcacctcttcaggtaagcaggacttcagtcctccatagagtaagggagcgggggcgaggagtccaccgcacctgccctggggctgctggatggaaggaggtggaaggctccctaatggaaacttacataaatactgtgcgctatgcagcgtcttcacaacagcccctctgttacagaaaggggtgacttacccaagggcccacactggggaagaggccaagctgggattccagctgtgtagcatcagcctcccgggcccatgctgtttcctctaagccaggcttcagcccatggccccctccgtggaggtgacctgcttcctttacgtgatattttaatcctgggcccttcagaaggtgaaatttggagtgggaaggtgaacgtgttggtcctattgaggtccaccttccacttgagctctggggaccactctggccctggaggacctgtcccctccagctcagctgagagtctgggaggcatcatgctttccttcctttctttttttttttttttttaaaaaaaacacatatgtatatttagaaaagaaattgtgctgtatacaatctgatgacttgcttttttttatatggcattgtttttccataccagttagtacgcatccagaatataatttttaagggctgcatagtattctggaatacaataatgtacctaatcccctaccattggatatctggattatttctcagcattttaataagaaaacagtatacttgtagccaaatatttacacttatcggaaattttcccttacaatgaattccaggaagtgtgactactggtcaaagagtacacacaattatttgactaatgtcaaatagctttctagagtaccttcagtaatgtgcacctcctttagcaccccagccctcataggcattgcctaatttcctgcatctttataaatactggcatcaatatttaaacatttttgtttctggtttgtaggtgaaaattatagatgttctacactgcagttctttgaccattagcaaggttgaacatttttttccatgacttgatgggtcccaaattctttcttgattgagatccataggaacagcacacagtctgcttgaggaagtctcattgctctgagtgtctctggctctttgattttactgccttatgctgctgaaagaggcagagagagtcccagagggaagcctggggctgaagggtgacctgtggagtcactgtgggattcccagctggctctgctgccagggcacaccaggtttttgcagggtctggcaggagggggcctggtccaagtatccttaaatagctccttctcttccctcatctctcccagacatgatggactcgtccagcttctccaatctggatctgagtgaagaagattcagatgacccttctgtgaccctagagctgtcccagctctccatgctgccccacctggctgacctggtcagttacagcatccaaaaggtcattggctttgctaagatgataccaggattcaggtaagaaacttctgcaatctctggggaacagagtcagagtcctagactgagctacaagaagggttggagatcactcatccaccacttcttttttttattttttatttttttaaacggcatcttgctctgtcacgcaggctggagtgcagtggcgcgatctcggctcactgcaacctccgcctcctaggttcaagcgattctcctgcctcaacctcccaagtagctgggattacaggcaccagacaccacgcccggctaatttatttatttatttatttatttatttatttttattttttttccagacagattctcgctctgatgcccaggctggagtgcagtggcactatcttggctcactataacctccgcctcccgggttcaagtgattctcctgcctcagcctcctaagtagctgggattacaggtgtgggccaccaagcccggctaatttttataattttagtagagacggggtttcaccacgttggccaggctgctctcgatcacctgacctcgtgatccaaccaccttggtctcccaaagtgctgggattacaggcatgagccatcgcgccctgccctaatttttttatttttagtagagacggagtttcgccatgttggccaggcttgtctcaaactcctgacctcaagtgatccacccacctcagcctcccaaagtgctgggattacgggcatgagccacagcagccagcctccattgcttctttaaaatagagattcagaccctaccctagacctgcgaaatcagaatctctggcgtaggcccagaaatctgtatttagaaagtgcagcctgtcttgcgttactctgcaggccagcactggagagctagtccatccccgcactttctggatgatggtgtggaagcccagagaggtccaatggccagccaggatcccttccaggtgttggagccagcatgtcagagccaggcctagaactcccagctcactgctgtgttcactccagctggcttgactggaatcctcatattatctctttaaattcaacgatatgattcctccacaccccaactctgagagcagaatgaagtgatagagagaagggcttggccatgtagacttgtgaaacagtctaggaatcctggagagagataggtttactggcatatatgaccctggcatccttcaccaaaatgtacatttaaagaccatttcctggctgggcacagtggcacatgcctatcccaacactttgagagactgaggtaggaggattgcttcagcccaggagttccagaccagcctgatcaacatagtgagacctcttctctacaaaaaaaaaaaattataaattagccaggtgtggttgcacatgcctgtagtcccacctactagggaggctgaggcaggagaatcacttgagcccaggaggtcaaggctacagtgatccatgatttcaccactgcactccagcctgggcaacagagcaagaccctgtctcaaaaaagaaaaaaataaagaccatttcctaaccatactgatacatttttgccaaaatatataagtataaggagttctactggagaagggatcctcctttatcaattcattcatataaatttcattcatttattcctatgtttcattgttttaacactagtactgtatataatacttatatttaaatactcatgcagtgttaattttttttttcttttttgagacggtttcgctcttttcacctaggctggagtgcaatggcgcgatctcagctcactgcaacctccgccttccaggttcaagcaattctcctgcttcagcctcccaagtagctggaactacaggagcgtgccaccatgcctggctaattttttgtattttttttgagacagagtttccctcttgttgcccaggccggagtacaatgacgcgatctcaacttactgcaacctctgcctcctgggttcaagcaattctcctgcctcagcctcctgagtagcagaaattacaggcacgcaccaccacgcctggctagttttgtatttttagtagtagagttggggtttcaccatcttggccaggctggtcttgaactactgacctcaggtgatctgccagcctcagcttcccaaagtgctgggattacaggcatgaaccaccacgcccggccaatattctttttttttttttttttaattgaggcagagtctcgctctgtcgcctaggctggagtgcagtggcatgatctcggctcactgcaagctccgcctcccgggttcacgccactctcctgcctcagtctcccgagtagctgggatgacaggcgcccgccactacgcccggctaatttttttgtatttttagtagagacagggtttcaccgtgttagccaggatggtcttgatctcctgacctcgtgatccgcctgtctcggcctcccaaagtgctgggattacaggcatgagccactgccggccaatattcttaaaacaatagagtattgacacatttaatagatgtgttgggaaatggctatatttatgtatatttgtatcttcattcttccccaaagttcatttggtatattgccataaaatacataaacaatatggttgagaaagagaagtaaatcaatctcaagcaatgttattgtctttcaagtagcagcaatagctgtatttacggcagaaggggcaaaatgcttcctagatactaatgctcagattcagtgctggaatatggggagctggaaaatgagttaacattgccggctctggatggaaacagattatgaggtgccatatattggtgtatggccctcttagctgtgtagaaaagccatgagttattgccgaaattaatgccttgccagtgagatgatggtcattcacagagctaaacccagaactttccagtttgtttctgccctgagaaaactggctctgtgttattttatgcctctcaccaacccaaataacagaaatttttcgatgctcttcccctggaattaatgtgaaaatggtgaagaagagaaaactggcagacagtgctgagacactcctactgcattgcacattttggttgtagtgataggagcgagggcccctctggcaggcaggcaagcaggagcaagcgacagatcttgtgtgagcccggcattgctctgagcccagggtttaccctcagaggctttctttggaactgacaacacattccgtagagcacaagttccaactccctcctccacgcttcacgttactgtttgcaaactccacatattccaaagtcttgttttgtgtaaacagctagggaaaaaacacagaagcactcggcttccatcctcatgtcaggcagcagtccttgtctacacaggcttcatccttccctgctctagtggggagtaggacagagggcccccacggccctttccagatacagtgattccaggttcagtgattccaatggtggctgagattgcatacacgggaaagctgccctaaaaagaaagttactcattaaatcggattactccaatgctgcccccttcactaaggaaccccagcctccaatttctcccatgctcaaggcccctatccattgccctcccacatgtaccctgacacaaatagcactactctcagtttctcttcccgaggttaagttgaagtctgccctcttcctttcatcatgttctcctctgtcccctagtctgtccttgcaactcatggctaaagtgaggtacatatggcaggtacaggagctgcccagccattgatgcaaaatgggttaaactgatcctgaacatgctagggttggcttctctgtcttcagtatgacttgagaagtcccagagcagaaggtatgccaatgaaaatggagcaggccttgctaagagagcttgcagggacactggtatggacgctctctgtgactggacagaggtgatgctgagcctggactggagcccaaacctaggctccaactgggcccagggtgggccagcccagtagctctctggttctgctgctttccacacttaggggttctattgttccaagacatagaagaacagtggctgcatccctggtggctttgatcttgctgcctgcaggcaggggcggagggtgtggggaaggcaggatgagacttctgtgtgggtgtgtgggggcacaggatgagtctccagtgggggcatgagaccaacgtggggcagggctggatgggctgttcttggtgtgaactgtgctacagtgtggccttggcctgctctctcctcccatctctcctcccttagcctctcagtctcaccccacaaatctccctccctgctggcactgcaaatgaaatgcatggaggaggtggcatcagcagcagcatctaaatggccaagagcagccttagatctgaggacttgggacccccaggcttcactgacaagtaaagtacacaagagaccaggccacataagctgcagccctgccctcttctgctaaatgccttcaccttcattgccatttccatacctagggaagagccctgggggttatcatgcttccttcgtgctgtgccctttctccacattcatttcttcatccatccaacaaatagctttcttacataaactgtgagggacaaaagttgttgagaagacagtccctggcatccagggatttggtgtctgatgaaagagatagacatgtaaacaattgctgcaaggagataagagccctgctacaagcctgctggaggtaccgtgggaatgcgggaggggaggggaggggccgggctctgcctgtgggggtgggaaagacgaaacagcaacacaattccagtcacatctcgggtgcccagaagcggctgcaggcgtgaagacagaggagggtgtaaatgtcacacaggtgagggagggggaacggagctgtgctggtgcctatcatcgtggaacaagctctcttatggcttcccctgtaactccacctctactcccaccacgccaccatccctggtcctggctgtgagctgtatttagaaaggccctgtatttccaggctgtgggcagtcatttggggtcgttttgggtttgtgttcctagcagcaggatgtctggatcacaaaagcagtgatcccactaaaacctctctggcctgcatccatggaagatgtgttcactgctggccatctgtttgagtggggcactgaacaaaaggctatgtataaagttattcctcccagaattacctatgatagcaaaaattggaagcaatcaaaatgttcaaaaatagataaatggaagactggctcaataacttagagtatactgttatgatgtaacatcatgcaaccctctaagaactgggctaaataattttgagtgatataggaagtaggataaagaattagtataagctccagtatgttaaatgtatactccctctctctatgtatatttgtgggtatatatatatttgcatagaaaaaagacaggaaggtgccaggcacggtggctcacacctataatcccagaactttgggaggccaaggtgggcaggactcacttgaggtcaggattcaagagcagcctggccaacatggtgaaaccccatatctactaaaaatagaaaaattagccaggtgtggtggcacatgcctgtagtcccagcttctccgcaggctgagacaggagaattgcctgaacccgggaggcagaggtttcagtgagccgagattgcaccactgcactccagcctgggtgacagagcaagactgtctcaaaaaaaaaattaaaataaataaataagaaaaaaagataggaagaaaatacgccaaaatgtgaagtgtggttatgagcaaatttaatttgtttttacatttttatgtattttccaaagatttgataatgagtatgttttacttgtataatgagtcaaaacaaaaatggggatggtgttcatttttgtgttttaaatgtggattgagcatctagagaaaagtgacaaggatggtgagatgttagacattgtgtcattagactatctgaaggaggacacggcagttttcctttttaaaaaactccattagttatggttcaaaggaagtcccatggctagtggagaagtctggttctgggtttattgagcacaaaactgtaaaccacaaatgagtgtactatcacgtggattgtagctcaatagaagaggtcattaatctcggcgctaacaaccccatggctgtgtccagaggccctgagctccctgaccctaggagagtcctgcagaggttatgtaggagccatctctaagagttcctaagaggggccctccaactctagcacgttgtgattttttttcaatacagatcctttgctggccatcctgatcatgcaagccttctcatttcccaccatctatcacccattgatacaacactctcatcagttaatatcagcttcccatctttatatataaacatgcagccattgacggggtgacagcctatctgcaggatatccaggaggaagtagacagtcaggaagagaaagggagtaaaagccagaagcaagctgatttgtgagccctgccttttcctcgccattgttcagacaagcccattcctgactcagaatagtggaactagtcattggcctctcaaatcatcaacgcatctctattgatcatcttgtgctgacggctcaatggtcagtgtgtgggcaacagtaaggtgattaagaggaggtgctggcccccaagtaacttacaaacaagagtagaaaacaagtggccgggtgcagtggctcacgccgtaatcccagctccttgggaggctgaggcaggcagattatctgaggtcaggagttcgagaccagcctggccaacatggcgaaaccccgtctctactaaaaatacaaaaattagctgagtgtggtggcaggtgcctctaatcccagctactcaggaggctgaggtaggagaattgcttgaacctgggaggtggaggttgcagtgagccaagatctcaccactgcactccagcctgggcaacagagcaagactctgtctcaaaaaaaaaaaaaaaaaaaaaaaaaaaaaggagtagaaaacaagcatgtaaagagcagaactggaggagacgggcaaaataagagcaccagcaatgttcaaggcatcacaatgacatggccctaactgtgctaaagagtcagaaggtgcggggctccagtggaaaacacagtggattcaggaccagaaaaacaaaatggagaattagaagggtattcctggctggagctgtagtatgctgaaaggcgtggtgatggctgggtgcagtggctcacacctgtaaccctggttctttgagaggccaagatgagaggatcacttgaggccaggaatttgagaccagcctgggccacatagtgagacaccatccctacaaaaaaaatttaaaaattagctaggcgtacacctgtaatcccagcactttgggaagcctaggcaggcagatcacaaggtcaggagatcgagaccatcctggctaacactgtgaaacctcgtctctactaaaaatacaaaaaaaaattagctggccatggtggcgggcacctgtagtcccagctactcgggaggctgaggcaggagaatggcgtgaacctgggaggcgaagcttgcagtgagccgagatcgcgccactgcactccagcctgggcgacaaagtgagactccgtctcaaaaaaaattagctaggcgtgatggtgtgcacctgtagtcccagctacttaggaggctgaggcaggaggactgcttgagcccaggagtttgaggctgcagtaagccataatcattctgttgcactccagcttgtgtgacagaacaggacactgtctctaaaaatactaataaaagaaattagctgagcatggtggcgcatgcttgtagtcttagctattcggaaggctgaggtgggagactcacttgagcccaggagtttgaggctgtagcatgctatgatcataacactgcactccagcctcagcaacagagtgagatcctgtcacaagaaaaaaaaaaggcacagtgagaacacacagcatctgaatgtggagtggcatgatgctgctagaatggaaggttggtggagtttaccatgggaaagggagttggaatgagaggttggagccaagttagggagtgtgggcctgatcctagggtgtttgagagtcagtgaagatttctgagtgcaagaggacattgacctgcagcagtggtaagaaagatgaatgagagactggagagacacaagtctgggattcctgtaagaggctattggaaaagacgctggagctctgaaccagggcactcctggtaggagtgggaataagatcacgggcttgagagacatcctagagacagagttggctgaatttactactgagaaggactggaggacagtaggagggagaggaggagaaaacttgggtgttgttgaagataccatggccctaccttttgcagaccaccatattcatgcctcaatgctgggaacttccctgggctccgtttcctgtgccccatagctctggctgcttggtctctgagtttttcttgtcttagtaactgagctacttcacttctggccttggcacatgatcacagtgttgaactgttctctctcaaggaacactcttgtcccttccagccagcctgcaagctcctcgatgaaagacccagggtgcatgttggtgcccagcaggtgtatacctgtcaaagcactaaactgatttgtgtggcttgaaggcgtttactggtaacctgacctccttccctccctcccctgcagagacctcacctctgaggaccagatcgtactgctgaagtcaagtgccattgaggtcatcatgttgcgctccaatgagtccttcaccatggacgacatgtcctggacctgtggcaaccaagactacaagtaccgcgtcagtgacgtgaccaaaggtatgcctagactccacctcctggggagtctttttcagctcccagattctggctccacccgtcctggggtttggctccaatcagatacatgggagggagttaggcaccaacagggagagaagggcgagggtcagacccatggggttggaggtgggtgggcggctcctcagctctgcccgcagtacctggccattgtctctcacagccggacacagcctggagctgattgagcccctcatcaagttccaggtgggactgaagaagctgaacttgcatgaggaggagcatgtcctgctcatggccatctgcatcgtctccccaggtatggggccaggcagggaggagctcagggacctggggagcggggagtatgaaggacaaagacctgctgagggccagctgggcaacctgaagggagacgtagcaaaaggagacacagataaggaaatacctactttgctggtttgcagagcccctgtggtgtgtggacgctgaggtgcccctcactgcccttagctctgccttgcagagtgtgcaggcgattcgtaggggggattctgaggaactagataagcagggttcctggggccacagacaggcctgcgcattcccaatactcaggctctgctcttgcgtgaactgggctcaacattcctgttatttgaggtttcttgcgggcagggtacaaaactttggagcctgagagatggttctgcctatatagtttacctgattgattttggaggcaatgtgcagtgacccttgacctcttccgctggttagaggtgagaagagggagaaaaggccgaagaggaagttattgtgaccttggggacatgatgtcggtgatgaggtccaaagaggggcggccctgcctcagcctgtgctagtggcctgtgcccagggatgctttcctggactggaggctcaaggaatggagatgggctcctctacccctgcccagccagccttctctcattcattcatccacttagcaacaatttattgagcacctattaggtaccaggcactatgctaggtactggggttcagcagcaaatgggacacaggctcctctcccatgaagcttaggaggaaacattaaacaaatgttatttaattattaattcctaacaaggcaagagttttaaaaataaagtaagtgatgctacagaagggtagaatagaaggagggaagctgacgtggtctgggctacagaggtagagtgttgccaggaatggccttttggaggaagaccttttaagctgttatccaaaggatcagtaagagtctggcaaagatagcagagcagagttccaagcagagggagcacagatgtgaaggctggtggccagagagcatggcgcatcgggacgctgagggatggacagagcatggacagggagcaaggccaggcagggacagggccaggtgcgcccatggaaggacctaggtctggatcctaaatgcacggagaagtcactggagggctttggggccaggcagtggtatcaccggtcagcagtcatagaggggtggcctagggggtgctgccgttgagtgtctgtgtgggtggggggtggtgggattgagcagtgaggggcccagctgagagctcctgtgccttcttctctatccccgtgcccacagatcgtcctggggtgcaggacgccgcgctgattgaggccatccaggaccgcctgtccaacacactgcagacgtacatccgctgccgccacccgcccccgggcagccacctgctctatgccaagatgatccagaagctagccgacctgcgcagcctcaatgaggagcactccaagcagtaccgctgcctctccttccagcctgagtgcagcatgaagctaacgccccttgtgctcgaagtgtttggcaatgagatctcctgactaggacagcctgtggcggtgcctgggtggggctgctcctccagggccacgtgccaggcccggggctggcggctactcagcagccctcctcaccccgtctggggttcagcccctcctctgccacctcccctatccacccagcccattctctctcctgtccaacctaacccctttcctgcgggcttttccccggtcccttgagacctcagccatgaggagttgctgtttgtttgacaaagaaacccaagtgggggcagagggcagaggctggaggcagggccttgcccagagatgcctccaccgctgcctaagtggctgctgactgatgttgagggaacagacaggagaaatgcatccattcctcagggacagagacacctgcacctccccccactgcaggccccgcttgtccagcgcctagtggggtctccctctcctgcctactcacgataaataatcggcccacagctcccaccccacccccttcagtgcccaccaacatcccattgccctggttatattctcacgggcagtagctgtggtgaggtgggttttcttcccatcactggagcaccaggcacgaacccacctgctgagagacccaaggaggaaaaacagacaaaaacagcctcacagaagaatatgacagctgtccctgtcaccaagctcacagttcctcgccctgggtctaaggggttggttgaggtggaagccctccttccacggatccatgtagcaggactgaattgtccccagtttgcagaaaagcacctgccgacctcgtcctccccctgccagtgccttacctcctgcccaggagagccagccctccctgtcctcctcggatcaccgagagtagccgagagcctgctcccccaccccctccccaggggagagggtctggagaagcagtgagccgcatcttctccatctggcagggtgggatggaggagaagaattttcagaccccagcggctgagtcatgatctccctgccgcctcaatgtggttgcaaggccgctgttcacccacagggctaagagctagcgctgccgcaccccagagtgtgggaagggagagcggggcagtctcgggtggctagtcagagagagtgtttgggggttccgtgatgtagggtaaggtgccttcttattctcactccaccacccaaaagtcaaaaggtgcctgtgaggcaggggcggagtgatacaacttcaagtgcatgctctctgcagccagcccagcccagctggtgggaagcgtctgtccgtttactccaaggtggggtctttgtgagagtgagctgtaggtgtgcgggaccggtacagaaaggcgttcttcgaggtggatcacagaggcttcttcagatcagtgcttgagtttggggaatgcggccgcattccctgagtcaccaggaatgttaaagtcagtgggaacgtgactgccccaactcctggaagctgtgtccttgcacctgcatccgtagttccctgaaaacccagagaggaatcagacttcacactgcaagagccttggtgtccacctggccccatgtctctcagaattcttcaggtggaaaaacatctgaaagccacgttccttactgcagaatagcatatatatcgcttaatcttaaatttattagatatgagttgttttcagactcagactccatttgtattatagtctaatatacagggtagcaggtaccactgatttggagatatttatggggggagaacttacattgtgaaacttctgtacattaattattattgctgttgttattttacaagggtctagggagagacccttgtttgattttagctgcagaacgtattggtccagcttgctcttcagtgggagaaaacacttgtaagttgctaaacgagtcaatcccctcattcaggaaaactgacagaggagggcgtgactcacccaagcatatataactagctagaagtgggccaggacaggcccggcgcggtggctcacgcctgtaatcccagcagtttgggaggtcgaggtaggtggatcacctgaggtcgggagttcgagaccaacctgaccaacatggagaaaccctgtctctattaaaaatacaaaaaaaaaaaaaaaaaaaatagccgggcatggtggcgcaagcctgtaatcccagctactcaggaggctgaggcagaagaattgaacccaggaggtggaggttgcagtgagctgagatcgtgccgttactctccaacctggacaacaagagcgaaactccgtcttagaagtggaccaggacaggaccagattttggagtcatggtccggtgtccttttcactacaccatgtttgagctcagacccccactctcattccccaggtggctgacccagtccctgggggaagccctggatttcagaaagagcaagtctggatctgggaccctttccttccttccctggcttgtaactccaccaacccatcagaaggagaaggaaggagactcacctctgcctcaatgtgaatcagaccctaccccaccacgatgtggccctggcctgctgggctctccacctcagccttggataatgctgttgcctcatctataacatgcatttgtctttgtaatgtcaccaccttcccagctctccctctggccctgccttcttcggggaactcctggaaatatcagttactcagccctgggccccaccacctaggccactcctccaaaggaagtctaggagctgggaggaaaagaaaagaggggaaaatgagtttttatggggctgaacggggagaaaaggtcatcatcgattctactttagaatgagagtgtgaaatagacatttgtaaatgtaaaacttttaaggtatatcattataactgaaggagaaggtgccccaaaatgcaagattttccacaagattcccagagacaggaaaatcctctggctggctaactggaagcatgtaggagaatccaagcgaggtcaacagagaaggcaggaatgtgtggcagatttagtgaaagctagagatatggcagcgaaaggatgtaaacagtgcctgctgaatgatttccaaagagaaaaaaagtttgccagaagtttgtcaagtcaaccaatgtagaaagctttgcttatggtaataaaaatggctcatacttatatagcacttactttgttgcaagtactgctgtaaataaatgctttatgcaaaccaatttgccttatccttataaggaccttatgggagatgaatcattattacccccatttgacagaaaggatagcttgagcaatgccacactagcaagggatgggatttgaaccttcagcagctaggttcagaagccacaaattaactgctacattgtcctgcttcctattgagttgggggacctgacagacgactgatggtcttgctagctctctcctagagaggagataaaagaggttcccattcctaaagcaggccctgagccaggaaaattagaggtgctggaccaaactgtgctctactcccaggaagtgtgcagtcaatatatgacacctacgtgagaccctcaaaaatgaaaaccaaacagctactggcaaaactgtgtctgccattagagatggcggctgtgccagtgacctggaggattacaaatgactgctgtgcagaaacaggactcctaaggggcccaacttatgccgatgcactccattctgcttcccaaggaagtggggtttatgatgaagggtagcattgctaggcacagtaaacaagaacacagcattgtgatctgaaaataaggaaatcatgccagctaatgtattgattgaggataagttggcctggggatgtgattcactctaatttttcagaaacatctgaaaatatttcaaaccaaaggctaaaatgtgtttcagtgggatgagatggacttaggggaattggggttagaacttgagggttattttgtgaaacatgaagggacttagagaaaggaaatcaacagctgcataaatgggcatgtctctggctggagaaatgtggagaatggagttctgatacactgttagaaggatcttatgtagcatttttatagctgacctagaagaacacaaaatttccaaggctgtgttataatgcgcttttccaggtaaaccaagaggaatataccccaggaaggttgcataattaggatcaagtgttttcaagttttcatattccaagctttggttctatgcctacactgttcaatccagtagccactagctacatgtgagtatttaaatgaaataaaggtaaacatctagcttgtcaaccgcacaagccacagttccagtatttgataacctcagggctaccgtaagagacagtgcaaatacacaacattttcttccttttttctttcttctttctttcttttttctttcttttttcttcttttttttttttgagacagagtcttgctctgtcacccaggctggagtgcagtggcacaatctcggctcactgcaacctctgcctcccagtttcaaaccattctcctgcctcagcctcatgagtagctgggattacaggcacctgacaccatgcctggctaagttttgtatttttagtagagacagggtttcaccatgttgtccaggctggtcttgaactcctgacctcaagttatctgcccgcctcagcctcccaaagtgctgggattacaagcgtgacattttcatcatcgcagaatagtctatggggcagcactggtctacacaatgcattcttatctggtactaattgtgaatgactccatgaggatgctggcgtcatgtgcttctgttgatctgtagggcagaatggccactaacttgacatcatatggaagtgctatagggaacatcctccccttacaatgggctatgccacacctggggtagttcgaatgagtctgcttcttaaaagagacataaagcaaaaacactgcacagaccatggggttgataggctcaaagcatcatgtggtataaatagctcactggtgtgctaggagtattgattcctttagccctggagcaagcaaacagggcctgccaggagtgaccacagcccttcaatttccccagcttctaccaggctccttgcaggctgcctgtgcagtgcaggtcggtctgcctgccccatggtccctgcagatgacaagaaggatggatgctgtctgacacctccagcatggccaaggagatggctcatcatgctgacatcctataggcaactagtcctcattgtgggcagggagcccgtgaggctgatggggagtctgtgctcctcaagacccagaagcacagcagggtgtggagcctgtggctggcagggggaatctgagagctcgctgctccagacagctgctccgaatctctgtatgcacgcatgtgatatatgatatacgggatggtgttgcaagttgggttccagggacgtagactctgaaatgcaggttgaagtgcagggagcttgttagggagcagtctcaggattatcagccctggtggaagggaaagaagtagaattagcagtgggagaagttgggctgcaaagcagtctcagtgaaggtctcaatcaacccgtgtggggatctctgaagctgggatggccctttggattgccccaagatgaagtgagggagacacttctatattcctgcatcaggtagtcattggacgcaggctgttccctgaagagcatgtgatttcattgacatgacctcagctaggcggctcttttcagcctgtggcccataggacatgtccataaggggtgttttcttcacattctatacaacctggtgagcacttctggagtgagctgctctggcttggggagacgctggaagagttccaggccctctcctgtggctctatccaaggagagtgctgactccaaaggagggggttcccagcctcccctcagttatggattagctgggttatttttcctaaatcatcttgagtttcaccacgaggtggtgctactgccctacagggatagctttgagccacttgcctggccccccgcccccaaagccccaatcacatcccccttccaccctttctctatctccatgatatgagtgagattcagcaaggctctgagtctctgctactgagggcatctggtggtgcttacctctcctcatgccagcgacatggggttagggatctgctctctggcttctcctcccaggcaacagggagtatctgaccctctcacacctcacccaagggcctcccagggtcttgtgccccgggcctctgagcatagtccgtgctgacagtgagcgaggctgcaggttccctctgaggtccagccaagttatgtagtccttccagtgtttctaaccagccacaccaccgagctcagtgctggggatatggcgatgaacaagacagtcgccacccccaagaaaagcactgtgcagtgggagaagacagccctacagacagatgagtactaggcatgccaagtggaagctgcaaagtgttaacaagttaaaagtaacagttaacaagtaaactgctgtctcccaggcctggctctgccatttgctatttgtatgaccctgggcaagttacttaatgtttttgagcctcagtttcttcacagaatggtaacaataatagtacctacctcatagatttgctgttgttgtgatataatctgtgtaaagcatttataacagtacctggcacgtagttatcaatcatgtgttggccaacatcactattgggttgctgagatggccaaataagtttttatcaaggaggtgacattgctcccacattccgacctccgatccctgatgggccctctagattcactccaggctgattttttggggcgcctcttccggatctcagcttcttggtgctagggactcctgctccatgatatctaccagaactgagagaaggtgtcattgtaccacctggccaccagggggcaggtgtgccacttaataaaccaaattggccccagccgaaagctgcctggggagaaaagtgtgggaaagaggtgggaagataggaatataaatctgaaatgcatggaactctagtgactactgtccccaacttctttcagtcatgttctttatttgtcaatgtcaatccttcaacaatcaccaacacctggacttcactacatgcgtatttactgacctataattaatgtcagtattctctggcaaggggtggcttatgcccagagaaaagggaatcaagacaagtttcaagaatcgggtttgatcgattcatttatccccatgtttgttggaaccctgcttctctgatgaggtataaaccccaccatgatctcttcagtgtcatttgtctttgttccagacattgaaacttaaccgaccaccgtgtctgggtacgtgcagttctctgaacgtgcagctctgtggcttgcctctaaaccagagtttctcaagcatttgggtcctggcatccttttactctcttaaaaagaaggaccccaaagaacttttgtgtgtgtgggttagattcatcaatatgtaccattagaaattaaaacagacatttttaaagtgtattctttaaatgtggcaataatgaatctattaaaagttagaacaaataacacatttttttctttaaaataaccagtttccaaagtaaaactaatttaatgacaagacagtattgtttcacagttttgcaaatctctttaatgtctggtttagtagaaggcagccagattctcatattggcttctgcattcaacctgttatgatatcatgtgtcaagaagcctccggaaaactccactgtacacttgtgagaaaatgagagtcaaaaaggctaataatgtccctgctagaaaatgaaatgcaaacagacttatgcaaatttaaacaccttcctttcaacttcagaacctgtgaagttaattccatgtggcttcagaaacttcatactaactcctctccctaaagtgtctgccacatccccctcattctgtacttggctaattcgtgtgtgtgtgtgtgtgtgtgtgtgtgtgtgtgtgtgtgtatgtgtgaaagagaaggacagaaagagagagagatggtctctctgtggcctaggccggagtgcatgatcatagctcactgcagactcaaactcctgggctcaagcaatcctctcatctcagcctcctgagcagctgggaccaccggcacactcagctaatttttttgtgttttgtaaagacgaggtctctctatgttgcctaggctagtcctgaactcctgacttgaagcaaaccccctcccttcctcccaagccttggcctctcaaagtgctggggttacaggtgtgagccaccgtgcctggccaccaatttgtttttgactaaatgcagaatgtagcatttgtccctctttatatttatctgattttattatcccacactggcctgctcgaatgttttgaatcctcattttgtcatccagtattctcaccatccctcctgctcttgcatcttccaaaagctaggtctgcagaccatattcagagttgactgagacagagccaaagactgtgtcctcttgcaggttaacattagctcatgaaccagaagctctttggttcagttgttcaagacctcaccgataaggaagggaagcgggtgggtaatgagagactgagcctcaggtgaggggagtcattggaaagactcaggtaccacaggaatgcggggaacttgtcaatgctgccacagcccctccctcctcaggactcaacagcctcactccaagtctgaacagactctttaagaattagacagatctgagccaagggtcacagatccagggttatttgatctaggggatgcacaaggggtggatcaagtatcaaatttcaagctgggttgttctgcaaattgtctgagctggactctgggaaggaaggcatggcctcctagatactggttgctgagaggaggaaggcaggaagaagctcccgtgagctcctgggggtagtgggcagagctccccttagacctccagaccttggtggtgaaaggagaagtcacacatagcctggtcgacagcccttatttagctccgtagggcctccccgctgcactcttcacttgcactcagaagcctactgggcctctggaaaggcccatgccaccctgacccaatcccaaatcctggctggacatggtggctcatgcctgtaatcccaacactttgggaggccggggagggaggattacttgaggccaggatttcaagaccagcctgggcaacatagagaggcggcatctctacaaacaaattttaaaaataaattagctgggcctggtggcatgtgcctgtagtcctagctacctggggggccaaggtgggaggactgcctgagctccggaggttgagtctgcaatgagccatgatcgcaccactgcactctagcctgggcaatagagcgagagcctatgtcaaaaaacaacaaaaacaaaagaatcctgcagacacctgtgaacatctgtggcagccggcatggggctagggccaaagtgagggcaggtgccccaactttgtcaactcctgactactatttccccatttgcctactaggctggccagatagatcaagcggcctatcaggaagggcttggtcccctaagccactggcccagcctggcaaggactgtgagccccaggttaagcgtgtaggggaaatgctcctggtatcctttctgcttcctccacttcccctagccccactgatttacatgtctttccctctcctacccaggagcccccggagagggacagggaggggattgtggaggccctgggtgataaagtagggatgggggagaagtgataaagggggtgggggagaagccagctgcacttcctccctgatagggaccccaccccaagaggctcctgcctctgctgtgttcacaggaaggactggaggtggtagaggagtggatggggagggcggtgggcaggatatcagtggcttctgcaatcctttgccttggaagatgacagttcattattttgtccttcggttgttcattcatcagcaaatacttaccacactcctcctaccaccgctggcagagctctgggctgggagggccaaggaggcagattgagctcagtagctctgctggtctcccctccaccccactcttcagccttgagggaggcaatgagtgaataagagatgaactttaacagcaggtcattggaatcctgatagtgtctcttactgactgtgtaaccttgtgggaaatgtgtgagctctctgatcttcagctttctcatctgtgaatgaccatgtagagctggtaaagataacggaaggaaggctcgcagcacgccgcagggcctggccttgtctagggcagcaatgaagagcagcagaagtggcatctagcaggagcaggatttgaaccctggcagtcccctgagtcgctgtggaactgctggctatttgtctgtcttctgtaaagcaaagagagtaatggtacttacttcctaggtgttgtgaagaccacatgagctactatgtagtatttagaattgcgcttggcatggagttcggtgctcggtaaatgttagcctttattactgtaattttcatcatcctctggggaccctctcctctaaagagataacgtctaacctgatatcgtcttccttataagcagctcctccaacacattccaagtcagggatagacggggtagaagcagggattcacaggtggacccctcttgtgaaaggccaagctggccctcaacccagctcctaggatggcagacgcacgcctggcccggcccatggtgccaacaactccgcttactcccccagctccacggtgttgtgctggtgggtagtgcggaagacctgaggtctgccccattatctcctcctgcaaagcctcctccacccgttaacccccaaccgagattatagagcctcaactgtctccaactgctctgattcagggataaggctggaccttcgagcccatcagccccacagtgttgaggggaggctgcctggttccctgggagctggcgagctggggaagttggaccacggcagaggctggggacacccccctgcccacctgcctgcctgctctggtgttgcctccagaacttgaggaaaaaagtgatcctcctctggggagaggccttcctgggttcactaaggctcttttctcccatctctgtctttcctgtctacctcgctggctccaggctgcttctactgtttctacttctctctcaggctgctcttcccccacccccagagcctcgaagctgcacacctgtctcctcacatcagataacccgaccacagcagctcctgtcaccccagccaggctcccagcagccccccagctccctcgcccatcgccctcttcctctcctgccacccccaccttctagctccctgacacacctacacagccctggggagggactgggaggggaaccccaaagccttactgggtaatgagggtgtgagaagacagctcttcctactccccaacatacacgcgtgcgcgcgcacgcgcgcgcgcgcacacacacacacacacacacacacacacacggtggaaactctatacccctgtgcctagtcagaaacagtgacggggagggacccagggaccaggcctcgaggtatgaggaaaatccatcctgcccaagtgagggggttggggagtcacagaaaggggcagcttctggggctgagggttccactgcctcctgtttttaggcaaagagtttccagggcttcatctccaagtcctaaggctagaggcccccagcagggccctcctctggtggagctgggctggggaggtgggtgggtgccatgtggacctgccccagccccttcccactgttcctcaagaggccaagtggggaggaggttggggggtggttgtgggggcagcggcgctgagtttgggatgctggggctcagggagactgggtaagagctgggcaggacaccgtgggaaggggagtggtgggacctaggagggaggggaacaaagaccagggtgcctgggagctggaggttttaatacctcttgacaacaaatgaatcataggcaaattctgtggaaatgatggcgtaaagaaagatgtgttatttcaaggccccagtgtgttgttgtgggaagagagtggaacggggaggagcaggaggcctaatggcgcctgtaggtgggggcccgggtagagttcctgggggcgccgtgaaggtgatggcccctgcatctcatgtcaccttcagggggtgatgtgaaacagcaggtggccgaccacagcccgccaacccccaccccaaagcagagaggccctcatacccgctgagctcacctcactgtgatgctctgttcctcctgcagtcagccctgtcctctgggggctgtccccacaggcgcctgccatggcctgctcctcagcagtagtagaggagactatggagctccaggagcctcccatatatgggagggaatctgcagtgagcacctggccatcacccctcacggtgggctttttcctatcaggagaatgctgagttgatacccacagtgaccgcacctgccttgcggtcggagctcagtctattaagttgccagataaatgcatttgtggcctctggaaggttgtcagggcccttgctatagctgagacccaaatctcagcttctggtcaaccctcattcccgctctccgcaatgttaagactcattaaatgagaccaaaaccatatatacccattccaccctacataatcattacctatgacacatatttcctcttcaccttctcctccatgcctatatgttttttaatacagttgcaatcatggggtatgtaccaatctctattctcttgtgatgctgtgttgcatataaacatcttcctaacagcttcatagttttctattaagtggaaatatagtatcataatttacctccacaattttccaatcataagtgttcaaattgtctctaattttctatattataggcagtgtgatgggggacatcattaggcacatactccgctcctcctcccccaattttgactatagaaatctagtttaggatacactgggtggggttattgagccaaagggtaacagcacattatagctcttactatatattgccaaactgatttcaaaagaggttacgccattaccgacagcacacacacttcaccgagctctcgctaatgtttcaaaaaggcacctacctttgtctcattttaattggcattactttgaggacgatcaggttgaacatacccctacctctgcttgtgagttgtgtttcctctgtaccaggagagtctgtttctgggctttgaactttagtctccactgttctagtgcttcttaacagtttgtgtgagctctctttataagaatgattttagggccgggtgcggtggctcatacctgtaatcccagcactttgggaggccgaggcaggcagattgcctgaggtcagaccagcctggccaacatggtgaaaccctgtctctactaaaaatatagaaattagccgggcttggtggcgggtgcctgtagtcccagctactcgggaggctgaggcaggagaatcacttgaacctgggagtcggaggttgcagtgagccctgatcatgccattgcactccagcctgggccacagagcaagacttcatctcaaacaaaaacaaacaacaacaaaaaaatgattttaaagcattgtcaactgcaaccgtgtgcgagtctctccttcttgatattctctcccacttgggattccgatgcacttcctcttgcttcattgaaatctgtgactgctcccggcctgctccagcttccactcctcgcccatctgaagtctttgttttctcaggtttctgtatttagccatcttccctgccctccccttctctcctctcttctcccctgctcttgtggtgtcattcattcctacaatttcctctctcactgatgacctccaaattcttccctgagctccagataccaactgtccactcaacaaccccccttgctcatcccaccaacacctcaaactccacctgtgcaaagctccattctgcattttcctccaaatttgcttctcctcccatattccttaactcagctactcaaatacctttgggtttggagtcgcttttggaagaggaaagaggaaggtggaggctaatgcgcctgaagaggtaatcggattctatcaaccttctgctcagagaaggagcactgggaagggcaggagaaggccagttgtcagggaagatgctgctctatgcctcaatttccttgcctgcctggacactaaaacggatccaggtctagagatagatgcttgaaacccaaagggagtggtcaccctcctagagcaaattaacccttaacactggcctcaccagctttttccttcacctcttgagcctcttccccaataagagactggaagggatgaagaagaggtaaagctataagtctaaacttgtgctgtccatacagtagccagtggccacatgtggctactaagcacttgaaatgtggccagtcagaattgaaatgtgctgttagtataaaatatacactggctttcaaacacttagtatggaaaaaaaatgtaaaagatttcattagtaattttttattttgattgcataccaaaataatgttttagatttactatattaggttacataaatatactactaaaatgaatttcacctgtttatttttcctttttttgatgtggctaatagaaaataagttacatatgtggctcacattccgtttctattggacagcgcttatctagaccctcccagcaggtgaagtgtggggctccaggctcgggagacaagggcagtacatgacagacacaggtgcaggccctactacaggtgtgggccagccagagctccaaccagatgtctccagacagacactgaaggacagattctcagtggccagcctcactcaggcgaagcaaagccaggcaacatttcctggataattccttcagtctttgacttcatgagggtctctggactgaggcttagctctcctgagcttgtccctgtagatgccccattattctggaaaaacactcacccaggaccacccagacctttggatcccctcaccttgatagttcattggaagatttttctatcactccacctgggccaggtcctggatctctttccagaagcccagaggtgtccttcctcctgtctagagccctccctcccacgcactctgtcttaggcttagctcaccacaggctgggaggggatgagagatggaggtgtgctcaacttgacctcgaacttccatgtacactacctgggtcacaggcacattttgagctccatttctaatgcctatagaagggaacaggaggggacgaggggtggcagatatactccattgggctgcccttctctgtctcattctccttttcctgtgccccggctgcgccatttgagggcactgaagctggggcccagtctgcgtgggtctgcccctccctctccttgcctagatgactccacctctcccaggtcctggctgggatccgaaggccccaggcaagtgggggaaggggagccactcccactttcccagaagctgctgctgcctcttcctgttttgctaaaactctggctctggactcaagcaaagagggggaagtcaccagctcctgccccccgccccaaaaggtcaaccttgcccccctggtcctcttatttatagcccccactacccaccacccccccaccccccggccccaggccctggctaaggagctggatgtctcctccctcaaatagcagctgtggccattgccccagggatacagccagatgggcagatcactccagatgtgcagtgtttcgggaggggaggccaggccctttctgcagtccctgtggtactccccgcagcctggtcctggttcccctcaacttcaagacaacacttgaggatttcaggaggatcaggaaggttgcacctattttcccatttctgtagcctggccaagctaccctgtataccctagaaaggtcagcttgggcgataggccctactggattcatttgcataaatcagctcagtgctcactttgagacctcaaattatagaggaaaacaggcttagggagacagaagagacaggacactgtctctcttctattactgatgtgagcattggagctctgtccctaacagcccaatgtgtgccaggcactgtagttaagtacttcatgtcccaatctcatcggtcctcgcaaaaccctaatatagcaggcacttcataccctaatctgcagatgggaaaccaagactcctcaaggttaaataatatacgtaaggtcaagcagtggggaagcaacaagctccagcccaagcagctcgacaccaaatccttgctcttaaccacagtacaacacctcctcactctgtccattcttctgggtacttttaaatgatctctttttttctttctttcgttttgaggtggagtctcactctgtcacccgggctggaatgcagtagcacgatctcagctcactgcaaactctgccttctggattcaagcaattctcctgcctcagcctccggagtagctgagattacaggtgcctgccaccacgcctggctaatttttgtgtttttagtagagacggggttttgccatgttggccgggctggtcttgaactcctgacctcaggtaatctgcccacttcagcctcccaaaatgctgggtttacaggtgtgagccaccacgcaggcctaaatgatctcttaaaagctcagtgcagggggccaggcatggtggctcacacctgtaatcccagcactttgggaggccgagacaggtggatcacctgaggtcaggacttcaagaccagcctggctaacatggtgaaaccctgtttctactaaaaatacaaaaaaattagccaggtgtggtggtgtgtgcctgtaatcccagctactcgggaggctgaggcaggagaatcgcttgaacccggaaggcagtagttgcagtgagccgagattgcatcattgcactccagcttgggcaacaagagccaaactctgtctaaaaaaaaaaaaaaatgctcagtgcagggaaccctggagcccaggactcagggcctgcccaagggagatgtctgcacattgtctctgcctcctctgccttcccacccctagccatttagaccaagctggagtgagggatctgttgaggcagagatttctagaatcactgttctgggtttcctcagaccccaagcacaaaggagagcaggtgagggcagaactattgccccaacccacattgatcccaggatccattaggtgtctcccagcttggaactctggtgactctgaatcctccctctcctttgcagcatatgttattcgagtccctcatccaaccgttttctccactccaggttctgttcttagattgtatcctgctccccaggaggtgatgaattcctcactgtggcttttttttttttttttgcctctgtgcctgagagtctgacacggtgcctggcaaagagtagatgctcaacaaatgtgtgttgatgaagaggagtataatggtgatcatcatgggtggagtttttcaaggtctcagccagggcaccctcgctcacctcccagcttagccaccctcagctcattgctctctttcctgcctctagtctcctgactcacaggtgcataatccaaactcaccctaaaagacactcctggggaaagagaggtgagtgtcttcaggccaagaccttctcaggggtctctcctggagattctggagtcagaggtatgtgcttccaaagaagaagcattgagatttgtagtaaagtgggaacctgttgaccagccaatatagcccctggggcaactggagggttgtgttggggcatatggcaaggccgaagctgaagcctgccagtgggaggaccacagcatccctgcaggggcccagtggagcaagtaatggtgagctggctgcagaaaggcattttgcaactcagacgaatttcttctcactcatgattagagtatggcccagtggctgcctccctgaccataatctctgctggcacaggcatctctgggctgggcagcccaggctgtggaaggagcgctgacctggaaaccctggggcttgagttctctgcctgccctggccctgattaaccgtgtcatctctggcaatttacgtcctcctcatgtcctccgtttcctcatctgtaaaaggaatggtttggattagaccagggagtcccaaacttaaccacaccttagaattacctgtggtgctgcaggttcctgggtactatccaaccttctcaaagagtctgatggggtcggggatgggactcaggaatctttttttcacaggttctttcaagtggtttttatttacttatttattttttgagtcagagtcttgctctgtcgcccaggctggagtgcagtggtgcactacaaccttgacctcctgggttcaagcaattctcctgcctcagcctcctgggtaactgagattaccggcatgtgccaccacacctggctaatttttgtatttttagtagagacggggtttccccatgctggccaggctggtctcaaactcctgacctcagatgatccacctgccttggcctcccaaagtgctgggattacaggcatgagccactgcgcccagcctacgtggttttgataaagtgttaggtttaggggatcctagagactagataatcttataaaacacttgcagcttttgggagcaaatattctcgtaaatggaatgggtacatcgtgaggttttgctgctgaggggttttatcagagagcaggtgatgggtagtttattttatcattatcattatcattattattattttgctgctggcattattgttatagaaggatgtggcacctgcccatctgtgtgtttctgccatgaacaaggttctggcctaagcggggcctcgcttacctcagggttgggagtgaagctgggagaggagggtagggagggaagggctttccctggtgggtggggcctcttggcaaggggaccctgctttccctctctcctgggtggagtttggctccacaggctttggcaccaaactctggggctgagagcgtctctccccacccatattcctcccccactttcacatcttagccactcagtcctggggactgcccccttctactcttcctttcccgacttcctccccaggcccctccaatcctgccaacctcattcctgagcctggctaatctgtttgccaccctctgcttccttgcctcgcccttgcacatgccattctccctacctggaatgtcaccccctcttctctccccactctgattcacatctctgggctctttcaaaacccagtttgaccgacacttgccagcttcatggaggccgtgttctgtcccgatgtgctccggtcttccctctttgcgacgcactcatttgcgcttggaataatagaatcagaaaactgctttagactcagaaaaataaatatagatgatctaaagcagccccctcatttgacagatgagaaaactgaggcccagggtggttgcgacttggccaagatcacacagtgagattttttaaatagttggctttgcaagaggctttcaagtattttatgtgtttgttttgtttcctcaacgctctgtgaccaatagaagggcaggtgctttgtttttgtttttgtttttaatgttcttatctccacactcttgttgtgttgtgtatccaggagctaaagtacgcaattgtaatatttgttatgccatcaggaaaataattctaaaactcacagtggtacaaatcttttcatgtattattcatctgatttcaggccagccctgtgaggtaagtagccaagatattgctatttacaaagcaggaaactgagacttagaaaggtgtgacccaggaagttaatggcagagctgggcctaaaactcaagttccctgactcccagcccaggctcctgatccccacaatgtctcctttctagctctctcctgatgctcctggctggggacctggcatagtcaagggggtgaccgttgccgtcttttcattcatggtaccttctttgtcttgggcttgaggagactgtctggcttcctgcccctccccatcctgtcccagtgctttctccttgtgtcccctgccacatagttggcacatagacacatacatgtatattcagaactgcaggtgtctagggcaacagaccacaggggaaaccacatcacctttgttgactctgtagggagatgggggtaaggggaagggaccagcttagttctctccacctgccccagtccccacagtcatctaccctcccaacctgagaccctggtacttatagccacactgtagcctcttcttgatctagccatccaattctggcccagaaggatttggcacctcttctctcagatcctgacatcctagataggggtctgcctgcctgtaccccaaagtcacacccccaaccctactcagaatacttatacggttcccctgattctgctggtgcagtgcaacagagaaaggtgggaaacagggatgagaccacatggctggggtcagaaacttggcatctggaatgagatgtacagggacctcaaggaagcaggctacactggaacaggagaccctggcaatgcgtccttcattattttgacctgctaggaaattgcacaggctctgaatcagacatacttgggattaagtaacttactggctctgtggccttgggcagatcattttgtctttctgagttttactttgctcatggaaacaatggggataataatgagatttaagtgaaagggctttgaacagcgtgcgatatgtagaacatgctcaataaatcctaaattatctttccttccttctttttcttccactccttccttccctctctccttccctctctccttccctccctccttcctttccttccttccttcctttttttccttttcttccttcctttcttctttccttcctttctcccttcttcctttttttccttttcttccttcctttcttctttccttcccgtacttcttatatctcttatgtctggtcccatgaaaccctttatttggagttggggacctaatctcccttactaatctgcaagtcccaaaccctgagaattggcatttgattccagatccagaaactgaaatgcaagatccaaactaacccaatacaatgagtgtccactgagatctcactgaactgaggccttggggaaggagcactttctggttggctttgtgcgaagtgtgtgaggataagtgcaggctggcggaggaattgtggctatggcccccacatctagaaggttgggaacggagggtatcagaaagtttggataattttctggggctttaaatcctggcacaaagtaggcacccaataatatttgttgaatgaatgaatggccctgtcacttactagttgtgcaactttgatcaggtttcttaaccataaaggccatgctttactcatgagcataacaaggattattgttaagaataaatgatctaatgtttgtccaggggatagcaaggactgctacgcagtaaatggtaaacagtagctaactaaggaaagaaggagaggaaaaggagaagagaaggtcaagtaggcccagagtcccaaagggaagcagattggggtgccctcaggctttgtgctggccggagactctagggaaggctcaggaagccaaggcacgtcttttggggccagtctcttctgctaggtgttcagctgagaaagctatgagaggcctggactcaagagctttttaaaaaaaggctccccacggtaaccaggctagaagagagagaggtagcttgcatttctctgtctcatgtctctaaggttcttggaggctttctaatttatcctggtaggggacatgtgacttagaaactggcttaggactctgccatgaggcggggtctggtaagtaaatctggcaggggaaggagagacacagtccagctttcccagatgcaggttgcacatgctgtctttttctctgactatatgttggaggcagtgagtcctggtgtgtagaaagccacatgttgggcactgtgtggacagacaggcagggagatagcctcttaattcagatcactcaaaactcagtgatgagtttaaaaagagaaagaagactcacaaataaatgtatgcacaatgcacatggtagaaatgagcataaaagggcctggctttactacagaaatagctacagaaaagctacggatatgtgtgtgttgggggttgggatcaattgtggagctagacaagggagatagcaggcatgtcgctgggggtctgcagtaaccaagcagtttttctggagtctgtggacaccaaagaggcatttggaaggaggatgatctggcttggaattgagggctggggatgacatacctaaggtggaacagcatgtgtcttggagagtcagcatgggtagttatggggtgaaagaatgtactgtaggtgtccagctggagaagggttcttgaaagtcatgaacatctgtcccaattcaacaaataactcacatcaagtttgaattctgtgcaagacactacaagaaatgcacccatgaccaaggcagtagctggactcagggatatccactcttgaatgctcttggaaggaagagagttgagacaagtagactaggtgggcgaaggtcaatgtataaagaccacacagttcagggaagaattcctcttggctatgggaatccgggaaagcctcccggagaaagtggtatgccactttctttttcttccagtccttccttccttccttccttccttccctccctccctccctcccactagatagggagtgggtcttgaggattggaaagatttcaacagaaggagaaaggggctgggaggacatttcttttggaggcagcatgatgagcgaaggcaagggagtgtggaagcgtgaggacaggatgggaagcagctagcagcccagtctggcagaaataatgatagtaataatagtgactaacactactgagccctaagtaccaggaggcactgggcagggatgtgtatggagtgtggtgccaggttgtaggaggacttgaatgccaggaaaggcttttgggagggtaagttaacaagcgcagaggggttggatctgcctgccgaaggagaactgggacaaggaaaggggctgaagtaagaagtcccacgctgaatatgccggtagcaacagatagtgagtcccgagctgtgcctgagggaagggaatagaatctatggtttgaagttcaccgccagcatggccaagccctcctaggccactgtgggctcttgcttccaatcagacatgactccacctctggggctgcctcaatttttccttctctggattcttggggtttcttaggattgcgggagatagaatggggtcaggggaagctgctccttggtttacaatggtggcgcctcacgctgcagagagcccagtgttcctgcgtggtggacctctccccttcctaggagcagctccagattgctggaggcagacacggctgaccggctagcccaggaagcacaatggcgaggtccttagcccctgacccctgaatctgaccttctctccagctaaaagcggagggagaggcagggagggccactgcctaaagccggccctgagctgagtttattagctgagggagggctggaggcggctgcattccgactcacagactggaacatttctgtgatccgctgtaatgcactgggggacactgggcacattgctgaagtttgactcatagggaccgggagggggaaagaggggggttgtggagggagaggaatgggaggaagagaggaagaggagaggagggaaggaagtcccttgagaaatttctttaaaaaaagaaaactttcaaaatctgcaccacccccacaccctttttcttttaataggaacaggctggacccttccgttcccctcagcaggcatggtgtgtgtgtgggggtgccagtgggggagggctgggcagtgattcaaatcagatcctggaactttcctgaggcaagtcgtgcttatgtgtgtgtgtgtgtggtggggggtgtccgtctgggattccttgtatgggacatgggacaccacgcgtgtcccactgtgcccgcgtgtggctgcgtgcttgtgggctgtacgggtatgtagtgtgtgcacgggggtccaccccgaagccccagtgtgtgctgtgtaaatgattctgccccttgtaaacatggatgcgtgctcgtgtacgttgtgtatgcgagggtgcgtgcgcccaggtagctgggtttccgggaattgtgcacggctcggagcgcctcgcggcggctggggctcggagccgcggccggcgttccgggttcggcgctgcctccccggcagtgcgccgcgccccgccgcgatctggccgcgcgcggtgtctgcggcccgcctgtgcgccgcgcggtgcagtgtctgccggggtggtgtgtcccagggccgcgagcgcctgccccctccctcccctcccccttggcccgctctcagactcagataaagcatttccttccattgtcatcctacccggccggccgggctgccagggccctccccctcccggccccctcccttcctctcgccgtctcacagtcgctctgcagcctccggcgactggggggatgtgaggccggcgccccagccccccgccccgccatgagccccccgctctgagggccccggcccctggatgcacagccccggcgctggtgagtactgggcggccgccccccgccccgccctgcgcgcggcacccagcgtcgcccgcccaggctcggcctccggccctggccccggctccggctccggctccggctcgggctcccgccagccccgcgggcccagcccggcttgcgccccccgaccgcgcctgtcccgcccggctccgcggggctctggcaggctccgggtcggagctggttccgggggccggagggctcgggcctcccgcggggcgcccacatccgcccgccccggggcagctacaggcccaagggagggagttggaggccccggccgccggtgtgcgggccccacgtcccatcacccctgttcccggggaggacgagccgagggaccggggagggagggacggccgcggtggcccggcgagggggcggtgctccggggcggaaggtttggaagcgcgagggcaaagggcaggacccactgagttggggctgggctttgtggggctggacatcagcgcccccatcccgtggagccgggcagggccctactcagatccttcggcggggaaagaggtcccttggaggggtctctggctgggaggtttctctgagcattgccccccatccttgctcacccatcagggtgaagagacacctgtgccctggtgagctgtggaggtcaatgccggcagcggcgcgggaagcggggcgccgcaggctgtgtgaggggtgggggacaggatgtgcaccccccaggcctggggtctgttggtgttggggagtgtgagagcaggcggcaaatttggcggatgggctgggttcccacgaccagagtttctaggcctgtgggccaggagagagccctctggggcccgcaggccacccagtgccccacccccattgcttcccgctgtggctgcccgtttgggtctgacctccccagccacggctgctcctcactccttctccccgctggctgtcttgcgcacccaaaggcctccccctgcatctggacactggctgtctcgctgccaacgctccctgcccactcccctaggcaacatctgcctgaggcccagctctgcctgccgctttctagggtgagaatggggcatcggccctcagcggccctgctgggagctcaagatcaacttctcagcctgttccctggacttgcatctggacctgtgggaccttcctcaggacccctattcagctatagggctggcaggagactgtgactcaggtccctgccctccaatgagacgctttggcggccctttccccctcccaggactccataggctgctaggctgccgctacccatgggttattaagtcattttcttcttctctgcagtcatttctgagggaaactaaggcacagagtagtcccagtgagtggtacccagcccagtgaacctagactggaaccaggtcttggacctattgggactggtcctctgcctttagtcccctgatgtccctccttgcccagcagggccaggctcagaggcaagctcagagctgcctcctgatgttgcacctggaatggtcctggtgcagtgttctccagaggcctctgtgtcccttattcggcgggtgtctctgtggccaccgggtcttacagtgggttttggggcagggaagtttgcatgtctaagctgttcactccactttgacactgaagttcagtttccccatcagtaaaatggggagaaaattccaagcacacttctcagagcagagcagaagaggttgactatggagaggagaatgaaggtactgtgctattatccaccccctccagtttgcttagggataatggacctcagctttagtccttggtggcctcttggcatggaagctggaactttctcaattctgatataaaacccttgagcaagagagaaactcatgtgtgatggggtagggagtggggaccctctggtagctgtgggcaagcatgcccaaggacttggaagacagaagcctgtggaaccccatctcctctttgccttctttctcctggcctttcattaagggctggtaattttgaagtcggcaggggcctgagagaaggtgagctaatgcaaggcccagggtcacacagccagggaatggcaggcggagggggccagaaccaggccttctcactctgccagtgcccattctgctggcccaccctccacagcttggctctccctggcctgccctcagttagacttcggactgaggcctccagtggggtgtggggtattgctggaagcagggcctgaataaagtctggggctagcgggagggcatctgtcctcccggaggctcccaggactgtgcatttgtgtgtgttggatttgttcactagatggggcacttcctttggggcaggaagctttgatttcctgtggtgatggtgaggaggggttggtaaggattaggagtggggggctcagctctgggccatcctcgatgttgttgtttggaaccacggaggggcagagtttctgcttaagactgcttctcccagctctggggaggcaggaaggagagccttggtgaggaggcccaggccaaggctggagaaggaggctgtgagagcagtgagctgggggtgggcaacacagcaggaagcccggtcagcaggtgaccctgccgggtattgtgtttccttgtaatattttccccctgtttctgtggtaaccttccctggaggccccaggccttagggctacaggaaggtgcccccagacagccctttccttagaggcctgagtattaggaggagtccccagatctagccacctccctaacccttttggctccctaggatcaggggcctgggaatctaggcgagtggcctgagtgagcatggactcgcgggctgggggaaggccctttggcgtcttctggggggaagggagagggaaagaggggagctggacgcccggaagggccctgagcaacttgagtcgctggcctagcctcttgtttcctcccaacccatccccactgccctcctccagtctccctcctcctacaagggagaggtgggtagcatgctgcctggggcaaggggcaggggtgggggtggcagaggtgggagggatgttttcatcagcagagcatagctcctttggtcctggaccagttcccagaggcaaaataaattcaggacagcgtctgtttagtgtgggtctgcatgagatgtgtgtgagcacacactgggtgcgtggtgggtatctgtgtgtgtacccctccccccagaccctgtgtatgctgatgtggtggttccgggcaatttgtgatctatgtggtggttctgtgttgggtgttttgggaatatgcacacataaaagacagatgtgcatcatctgtatggtgcacaccttttcaggttatggccctgtgtggggtaaacatgtccatatgtatggacatgttttttgggggtatatatctgggcaggagtggctttttaatagaagtccagatgctggcttgtaaaacaccatctcatttgggattctcaaatgcgggttcctggaccatttgcttcagagtcacctggggaatgtgttaaagataaagatcctgggccgggcgcggtgacttacgcctgtaatcccagcactttgggaggccgaggcaggtggatcacctgaggtcaggagttcaagaccagcctggccaatgcggcgaaaccctgtctctactaaaaatacaaaaaattagccaggtgtggcgtgcgcgcctgtaatcccagctactcgggaggctgaggcaggagaattgcttaaacccgggaggcagaggttgcggtgagccaagatcacgccattgcactccagcctgggtgacagcaagactctgtaaaaaaaaaaaaaaaaaaaaatcctggctggacgcggtggctttgggaggctgaaacaggcagatcacttgaaggcaggagttcgagacgagcctggctaacagggcaaaaccccatctctactaaaaatacaaatttagccaggcatggtggcgcaggcctgtagtcccagctatttgggaggctgaggcagaagaattgcttgagcccaggaggtggaggttgcagtgagccgagattgcgccacctcactccagcctgggcaatagagtgagactgtctcaaaattaaaaacattcagatcatcaggcccattccagattgactagtcagaatctgaggggtggggcctgggaatccagattttaacaggtgcttcaggtgatcctggggcaaggctgtgtttgaggaccactgccctgggtcaacctcttcattttttcttttgaggaaatagacccacagaaggtatgtagtttctctaaggtgacagggccattaagaggaagaaccaagagaaaactcaaggcttctaagacctgtccaggactttgtgctcacccatgtaccccctgtgtgtgtgtgtgcatgtgtgctcataactacactttgtgatgacgtggacccgattgggctggtgctaattggggctgatctttcctctggggtcctggctttcttgttttctgaccttgttcttcttgctacattctgctgcctcctttttggagaaatcctggaactgttgagtgtcatccctaaaggtcaatgttggcaaccagggaaccatgttgctgagccagtcactcagcaattcatccattcactggatcctagctgagccttgcaatgttctaggcactgggtacagatatgcagtgaacacacaggcacaacatctgacctcagggaacttccactctcgtggagaagacaaacagaacaagtaggcaagtaaaggaatgagtatgtgtgactagttacacactgtgataagccctaagaaggaaatgaactgggcatggcaataaagaataacaaggtgggacttcctttgatagggtggccaggggaggactgcctaagaaggtgacattgatgccgtctcccacctccccaggttggaagtgagccatcagagcctctggagccgtgcttgtgaactccctggatgcaggtggaggccctgggctgaccaatggctaagggttggagctgaattgccagcctctcacttggcatggaagatggcagtctcctccccttaagcagccccaaaggttggcagaggggagagcagaagggggagctgcacaaggccaagatcaaagccactcccttgcttggggagcaccctttttcttccatggggctgagtccagggccctgtggccatactgaatgttgagaaggaggcagcatctccccaggagctctcctgtttcaccttcctcccacctccttcccttctccgccatctccccagttggggaactactgtgccaagtcccttccctctggtctagcctgctgcctctgtggccactttactttacttggctcaggaatcaccaaagagagggggtcaaggcctagaggggacctgggccctggaagagaggacagaaccagaaaggaaggctggggttgggaccagagaggacagggctcaagggaggatggaggctgggacaggagaggcacacgggaaaaacctgagcccagatggccctttggctgggctctccagagtctctcctgggcccttggaatctgctgcccaaactgggggagactctgagaccagagggccagatggggctgattcaagacacgggctctacatcagctgggaggaaatgcccctgtctggtccacctgcagctctacgcttcacccctctcaggaggttaactttcacatcagcaacttgggtgtcaggttccggctctaggccagcaggcctcagcccctctcacctccaacctcttctcccaggaaggtttttctggggacccagagcttcaggctgcaggaaggtgttgcctggcccttgccttagattattgagtgtcaaaggagccctgactccaggtccgcttttccccctgcacactgccaaggctgccttccaccctgggcctctctggaggctgctcagtgctcttcccgactcccaggccttcccgcccaagctccacagcccccacagcaggatggggaggggacttctcctcgtcaggaaggccctgggctaagctcaccatccccaccctgtctccgcaccacttcctttttcctccatcttgcctgcagggattgtaaaccctggtggtcagaagctttgggcctatagggtcagcctctcaccctggggatccgtaggggaactccaatccccttcagttcagcttcctccctgcaggacccattccaaggagcctagctctggctcctgaggtgaccccagcccagaagtcccattcccctctaccccaacccccagaatggcaggaggagcagggcagtctgggagtgacaggttcggaaagagtctggggtgggggaattcggcgattgggctgtaaccgtcttgttttgcttgccccacaaacctgcacgtccgggacgtccgctgcgccctcccaggcgcgttcaggttatagaactgctctcagaatgacaggctggtggcacccggctggggcggggggtggggaggtgaaggggggagccccaggcaagggggagctggagggttaaaaatagcagcagccttgtttcggttagcaaatgtggaggcggggagctggaggcggggcgggcggcggacgatgtttgcccgcagcgcagggctgtgcttcccctgggtcccgggagtgagtcacggcggagacgctgaggaggtgctggcgcagcaccccacccccaccgggcgaggggctgagcgcaggcctcgccccccagattccagcgctgaaggaggtgtgactctcccttccaggcagccggggccgccttgagagtgagccaggggcatcgtgtgataatagggaagaaatgtgccattctgacgccagagatctgggtttgaatcttagtcgaaaggtgtgggatcttgagtaaggatgctgatggctctgagactcgatttcctagtttcaagtggggcttttctctgtgccccacttagtgcttaaagcactctacagatgttaattatcgccttagaccctactgtatatatggcaaagcctggcaggagagcttcctgtaaggacgcagagggaggaatgggatggccttggctgagtctcggtttctcctgcaagtgtgattatgggcacattttgctctgtgcctccacttcttcatctgtaatgtgaaaataattacagtttcaactttatggagttgctatgaagattaaaggagataatgtaaataaagttagctctgtgcctggtatgaagtcagggctcactgaatattcatcatcattattaatgttattgttcatattttttgaaggtcagggatagggaatgttgcagacacgaagaacagaaactgggcctagccccagttcacagaaaaaaccaggccaaatccctgttccagattcctccacccatcgcggcctggaacagccagagagctgtagccagaagtcatagggtggacccctgcttctcttgttcactccctctcccagcctctcccagtgaggcagggcgaggagggaccccagggccttgaagccagctggccctggagaggggctgctgtgccagcttggggagggtctgggatggggctgcccctgatggccctgatgtggagtaccttgccagcatctgctggggtgaactttattttagcccttcccttgttgctcttatgaagaacagaggaggggtgggcaggtcagtgatgtcagcagtgagtattcccagcacagcggctctggaagaggcatgaggcatttctttcaggaaatgatcattattcagccagaaggcattcattaagtaagtcctgactttgtgcccagctctgtgttataggcccttggcgagactcaggaggggcagaggacgctaggttgtagataacacggaacctcagaggatatatggtccaagaagacccgggggcggtgaaaaccctgtggactaatgctcacgggagcccgaggtcacactttgactttgctaccatgggctgtgtctatgtacgtatatatgctgcgtaattattacagaggcagtccatgtgcattgtggatactcagacaggacagaccagcaaaaactaaaaaataaaaaacatcacagacgcatcagtcagagatcactgcacatctttccatttccgtatgtgtctttatgtgttgtaatttttatgaaaatgagattactctgcatatactgttttttcagtcagtgatttccaccttccattacagtaaattttcatcttaacggaattcccagttggccagaaattgtccttacagctggtttgtccaaatcagtacccaggaccatgccatctgtctgttgagtgacctgaaggcagtgtaagcaccctagctggatttgtgatctggaaaaaccccttcaaccaagtctcaagggcagcctggctgcaaaagggaatcaggtcacgtggagaatctgcccactagacttctgctgtcttccaaagtaataggcaaaaggagggggattctagggcaccactgggatgggacctctagggtatttactgtcattaatgttaatgtggcatccctgtgagtgtcaggattcacagcttgtggctattcacatccgagatgcctcagttcggtttgcctggttcttactgagtgcagaccagggtctccattgtggcttctgccttcctctgccccagatccagggatgctgatagccgtgcggctgtgttcctagtccccagaaagtggccctgaaggtaggtgctcccttttgcacttgtgggtgcttccagcgcttccacctgcccaaggcctgcccgtgagtgagagacagagatagagatagagagagagagagagagagagagagagagagagagagagagaccttttcctcccaagagaatttacccccgagtacccactctgagggtgacttgctctggttatgcaacccgcattttgtagagggcttgacagtttccagagagctcacgcagcgtttgatcctctgtggcagccctgggaggtttgcttgtttagttataccctgctggaggcagctaacaggaaacactgtaaagtgtttaaatggtacactttaaaatggtaaaatgtacatttaaaaaggaagggaaatagaagatgaaaaaggaattgaggatggcaggggtggaacacagcacactttgtgggaacaggctggcattgggagcataccctagggcctcactgtgtcttgcatccccttgtccccttggtggtctcacctcagccactactaggaggcaggctgagtaagcagtccacgccagtgtaccgatgctaagcttaggttctggagtcccagcagctccacataccatctgtgtgacctcagcaagtttctgaacctctcttagccttagtttcctcatttgcaaaatgagaataataacagtatataggccgggcgcagtggttcacgcctgtaatcccagcactttgggaggccagggtgggtggattcagtgagttcaggagtttgagaccagcctggccaacatagtgaaacaccgtctctaccaaaatacaaaaattagctgggcatggtggcttgcacctgtaatcccagctacttgggaggctgaggcaggagaatcacttgaatccgggaggcagaggttgcagtcagctgagattgtgccactgcactccagtctgggtgacagagtgaaattctgtctcaaaaacaaaacaaaacaaacaaacaaaaaaacagtatctgccttacagagttgtgaggactacttgcaatagtacaattaaaatgtttagtgggggcctggtgtggtggctcaggcctgtaatcccagtactttgggaggcagaggtgggcggatcacttgaggctgggagttcgagaccagactaatcaacagggtgaaaccccgtctctactaaatatacaaaaattagccaggcatgttggcgcacgcctgtaatcccagctacttaggaggctgaggcaggagaatcacttgaacccgggaggcagaggttgaagtgagccaagatcgtgccactgtactccagcctgggtgacagagtgggactccatctcaaaataaataaataaataaaaatcgcttagtgggaggatgttgccctccccccgccaaaaatgcctaggataatagccagcacacagtaagcattaacacatgattgttgatcattattaatattattaattaattaaaataatataactattgttgatattgtaacttaaacatttccctggcagtcttgggaatgggctgcgtggccttgtgggtggcacaggtgagaagagtgaggaaggcacatctcgctcggcctgcagctgggagaggcagaaccagtgggcaaagcaggagtagggtacacagaggggcagggtctgcatgaggagggcccaggagctggggtgacaaggagagcaagggaatgagtgtgacacctcagaggggagaaggagccgcccctagaggcagcctggggcggtgactgtgcttcctgttggggcaaacactggcccttgagaaagactggctcacactggctgctaaaggagcaactggtctaagtggggcaccagaggggtaaaggcgaaggggagagatgaaggcagaaactggccgacttatccccatgttacagatagggcctcaaatcttgcctgctcaggatgttttgcttatgataggaaagatggtagttagacatcatggtgaacttcctatctcagggactggagatactggagcagaggagtgaggatgaatgtggaacttatgtctttgagggctttttggaactgagaaatcttagggtagtgtggactaggggagcccactggctcaggaattccctaagtgggcagagcagtgcaggggcaggccaggctataagctgaaggccatgggtaataggaagtagatgcaggccctccaggcctgccaccgcaggaagtagctatgccctccccttcacaatggacttgcagggaaggctgagtggtggcccgagaggtcccgggagttggacagggctgggaggacccctgtttttctctgtcagaggacagcaagggtctccagagagtgccagacccagaaaccgacagccatacgcaactcagaatggcccagctccccggcctcaggttctgccaggtcttcatccttgaggagaggccacagggtggagctggggatctggggtgggggacaaagaaccagggaggatgctttcccacccccagggagctcaagccgctgctgccatggttacatctgcttcctgtttgattcatctcaaacagcagaagtggaggtggggtgggggggcgacactggctcccagctcaggccactgctgcgtggggctgtttacaacagccgcatgtgggattcccagaaagagactccaaaccggacatcctgcggctgcaaaatacccaggtgtcaagagctaaaaatagctgcctcagggccccagctgccctgaggtggcggagaaagaggcatgctcactgttgccacccttacatccagcctcctgtttggtgtgcaggagcttctctgtcctcttgcctcggactgatggaggccttcgtctgggtaggcagacatcccggctggctcacctggcagctgagcctctgccctgatgggcagctggctcctctgcctatacagagaaatggtgcagcttgctttgggacgctaatgagagagtcctagagggacttgggagcttctagagaagggatatggcagggcactgagagcaggggagttggagctgagggtcctctaagaccccaacccattgccagccccctgtttccagcacacagtcccaaatcattgcttggtttctacaaagtcaacagttgctccaaatgattctttgagattgtcttcatgggcacccaaagatgacatagcttctgccctctcacctgcgcagggctggggattgctgctggcacgggtctctgtggggctgtgtgattttttttttccatctttctgcatgcccatctaggtcctgcatgtgtcctttgctggctggcctgtggcgaatgggaatgctggggaggaggggtggagtgtattggttgagagcgtggattctgaagcccaattgcctgggctcaagtcccagctcaacctctgccacttgtctgtgacctcagacaagttatctaatactgctgggcctcagcgtactcatttatctaatggggataaaacagtaaatacctcataggtttgtcatgtggactagataagttagcaaatgtagggtacttagaacaactcctggcacataataaggtgttcgcttacaaaagttcaggtggaagagaaagggaagagagcatagttgggagaggctggggataagagatagggcagggggctgggcacggtggctcacgcctgtaatcctaacactttgggaagccgaggcaggcaaatcacctgaggacaggggttcgagaccagcctggccaacatggcaaaacaccgtctctactaaaaatacaaaaattagctgggcttggtggcttatgcctgtagtcccagctacttgggaggctgagggaggagaatcacttgaatccaggaggcagagtttgcagttagctgagattgtgccactgcactccagcctgggtgacagagcgagactccatctcaaaaaaaaaaaagagagagagagagagatggggcaggggcatgcattcaggagaaaatggtctgtggtagaggagggaggagagttgggagcagacactgaaacgctaatgccaggaaggaggctgcagacaagtacaggaggcaaactaatactcgctaccacaagggagcacccaccatgtgctgggtgcatcacagacagcattgctaatctcaggtgacctctttgaagtaggtattctttttgtttgtttttgtttttgtttttgagacggagtcttgctctgtcgcccaggctggagtgcagtggtgcaatctcagctcactgcagcctccgcctcctgggttcaagcgattcttgtgcctcagcctcccaagtagctggactacatgctccccgccaccatgcctgactaatttttgtatttttgttagagatgggatttcaccatgtcggccaagctggtctcgaactcctgacctcaagtaatctgcccgccttggccagggcagaacttccccaaagttctgggattataggcgtgagccacctcgcccagcctgaagtaggtattcttatctcctgattacaggtggggcagcttaagcaggttaaattgtgtttctggcttgctttgccagtcaccgagggagtcggggcttaaacccaggcccatctgaccgcaaagccaatgtcctgtgtgctttagaccttcaattccctcacagtttgtggcctgccagacttcatgtggcaggggcttccagcccactctcagctccctgctgggttctggataaatctgagcaagaagcattcagtgccaatcaatgagcagtatagagaatttctggaagggagacacaagaagctgttagaaagggcggcttccagggaagttctagggagtctgggatgaatgagaaacttatcctaacaacttttgggctctctgaattttttttagtatctgcaagtattgtacttgttcaaatatgtttaaggctgcaggctgtattctaaactccttgaaagtgagaaccaggtttcactcatatttccatcttttcaacccctagatcagtgacttcccagggaagtagtacctgcatttggggttgacctttgggtttcccctgtactggtctggcctggcctggctggaccactggctggctgggtggctgtgacctagccccttctttctctttgctcctctgtcaaatgagagtgttggtctgaacgatctctaaagcctggaagaggagcagatcctctgtgctcagcccccactctgtgtcagggaggcctggcaaccacagtgttctttctcctgtttatttgttcttggatcttcctgaagccatttcaccaccagccttcatcttctctgccagccccatggagactcaagctttttccagcctatgtcagggaaggagaaccagagacagcaacctcgggtgtgaagggagtcagctctgaacccaggactatggccttctgccactgcctgctttcctcttgctgctggggcctaggtcttcttgctgctgcttccttttccgctaatcaagagtccagggaggtgggaacagcctcaacaaagactttgaagatgagcggggaggatcgcttgagcccaggagcccagcctgggcaacaggggaagacttcgtctctacaaaaaaaaaaacaacaaaacaaaacaaaaaaaatttagccaggcatcgtggtgcatactgtagtcccagctactctggaggctgaggcaggagaattgcctgagcccaggaggtcaacgctgcagtgagccatgttcacatcactgcactccagcctgggcatcagaacaagaccgtacctcaaaaaaaaaaaaaaattaagaaaagacactggaggcatcgggaggggggcgttctaggtggcagtgctcctggggaagccttttgtcccactgaagacatgaagctcctgggagagcaggtggtcggcaaggctcaggttttcatccaccttttgcagatctaggaggcaaagtcatacctgctggggagggaggaccaagactggggcctgaggcaataaggtaggagcagtagggaggtcagtttgttccaggtgcttagaattgtgtttgtgtttactctggaggttgctgagggctggggtgcacctattggaacaggggctccataggtttctgggtaaaatcaggtgttctggtttaagaaggtgacttggtaggcccacatgccccagtgccaagtaaactgttcttaagtctgactgcagttgcctccaaagaagatagaaaaggggagtagccaggattccaaaaagaagagctctccaacctggcaaagagccctgtgctagacagtattcctggtaccttgggtccatttacttctcttttttttttttttttcaggctgaccccaaattagtagtaacagccctcggagagaggcagtgatgggaaaagaggtcccacactcaagccagaactgggaggcaggatgttcatgctctggcttcagttcagctactgacggggtgccagtagatacctttcctctctctaggacacaaagagagctgttcctgggtcttaatctctgtcgctctgcgctcttctgaacttgatggccctcagcacggggccagggagcgggggaaaagcagaactttttccaggaattgctattggaagcagccccgttgccaacacgcatgcacacatgcacacagcttttctggacagaccttatattatggattatcaccacaaaacatccctttggggcctggtagcccacaccacagaattcagggtcattaatttttctcctatccagagagtgcatggtgtccggaatctgtggttaccaggggagcaaggcacagagaacctggctctgctcccaagcatgaatgctgctgaccagcccctgggtagggactggggaggtgggacagaattcccaggaggcaggggactcagcctgacacagtctctgggagtgcaccctggagcccagtccagaaacctctgggaggaccaggtcctgcagaagaaagaagaggtactaagaaggcctgggattgggggttaccatgtcgtggggaggggagtttgcctctgaggcccaccaccttgagaaatatgcatgctgtgtctcaggcaggttgtcaggagagagtcattgtatttgatttgtcagctactaacagagcctgccacgtgccctgcgaggtgtttggtgcagatgggcatggcttccacagggcaccacagagatcatgatgaatgaagtgtcaaggtggtagacacagagcgagtgctgtggaaaaggaagaggggagaggtcagggagagcctcccaaggagatggacttgggctggacctcagaggatgggcagaatttaaataggtagagcagcattttcggaccgaactttgttacccaaagcatgggctggtttgaggggcagtcggtgggagtctccctgtgcccaggctgccccctcccccagcagcgcctgcctgcaggaagttctcatttctcggctctgaagaggaactgtggctgcctctgagacacagacccaaaggagcaccccggcagtttctcaaaaaatatcccttctgccccaagtctggccagcagagcgcttctgtgtccacccagtaccccgtccaggcagcctctgggtctcctggcctcctgtccccttgaccctgaactggacagcaagagggaaaggtgtctgtcctggacaggtggcctcaggactcatctctgtcttctccaaccccagctggcctccatgtcccctgggggctttctgctgctgaccagcttgggccctactataggttttcttgctgggcttaggagcctgagagaggtagccatttccaaaagaaaagatttctatctcagattatctgggaaagaggctgagtaggtcccttctctgaggaaacaggcagcaggacataggatggggcagtgggaggaaaagggtctgcactatggggtccttgggctgtgcactcctgaccttatcacttcacagttcccaccagatctgacttgacctccgggccatgacccagtccctcccccactctggaaacctctgtgtcccctcctgctcctttcactcccacctgggaggctctgagcaggccagggtccctctctccaggcctgctcctccctttctcctcctgtccccccagccatccccccagccaggctctcccacctctggccccacctcacctcttggccttcttctttccctcgggcgatgggagcctggtttggctgcccagggaagattgtatctgaccacaggagggagggctgagggcactgctgggtgagctgaggcctccttaggttcttgctgtagtctgagttcaagtcatttagaatgagtgacttgaggaagagggagctgggagcccttttcaccagcagggggactggaggagtcgaatggggtggggtcttctcgttttgattagcttctggtggaggtcccaggctttggcgtgctcaagcttggagtggcagggagcaggcctggcttgaccttctttccttcctgctccctctcctcacccctccctgcagctctttcactccgtctctctctctacagatgggacccaggtgagcccgggtgcccactactgcagccccactggcgcaggtaagagtcaaacccgggggagtccatggtagggagtggaagatgaggggtggaaaggctgtaagaacgcgagaagctgaggggttagagaagcagggtcgctggctgatctgccagagagccaggaggtggcggctccagggaggggcgaggagccggggtaagagaggcagctctggatgctggctgggcacagtgctaggaaacacaacaggaaaaggaaacacaggatgcccgtcttgtccttgctgggagcagtgaaacaggaaggaaagtaagaagctaatatttatactgagacccctaccccatgtcaggcaccaggcaaggtgtgttcttgtgtgtggactcggtcctcacaccggctctgcaaggtgggcatggcagcccttgcaggactgctctgctggaggggaagtgttctctcactgtctgcgcctcctccctctgctggcccgagcctcctctgctgctaggctgccctggggaaggactggacttcctgctgctgctttggtttaggacatgcccatggggccaggtctggactagacgcggtctgcccttcctttagtgtagccagtatcaaccaagggcctactgagtgcaagatatacagcctgatgcctaataattccatatagcagggagaaatggaacccaggtatcctccttgcttcagtcctggctgttgaaaagctaacaggcaggttagggaggaagcacacacaaatacaaagcgaaaaaaaatagaatgcaataatgtgaccagtgcccaatgagaggacactgattcatgagtttatccatttgttcaagaatcattaatgagttctgtctctgtgccagggtactttcctggacattttaggagagacactgatttattcattgattctatgtttgtggagtgcctgtcatgtgctaggtactattccaggtgctaggaataataaacaaagcatgaaaccaactccctgccctatggagcttaactcagacatggtacctgcccacagagcactaatcctggtgtggaacagccatgtgattggagtccaaaagagggagggatgctaggcgctggctgggtccagggatgagggaggactccagctgagctttgagcaaagagtaggatttggaaaagtagaatgtagggatgaggacagcacaggcagggggacattgtgagcaaaaggtcagagaacagaagagatgtggatttgtgacaggtggaagctgatggccaaggtggagggttcatgttcaaatgcagaccaagttgagagaagccaagttaggaccagattgtggagcctcacaaatgctaaagcacctcgaaaagtatttccagggctccaggcagagcccatcctgtattcccttctgctgtggaggcagtggccatgcagagtgtgaggggaagggcccctgctgggggtctgctcacagtccgtatgtggctgaagccactggtcttgggtccaggtcgctggccttggttcccccaagcccctcttgcataatcaggggagtcaccccgggaagccaggacacagaacatggaaggactgaaacctttcctggggcaggagtgttttggtattcgaaacccagactcaacccgaatgagaatccggatttcttgggaggcaatgtgaacaaggggtggggcatcaaagccatacattttctactgcgggcggcatctgggtgcacagccatcactgtccctccaggcctcttgatattcgggggacatggcgggcacctccttgctgggctcctgtcttgtgtccatgtcacagtcagggaatgttagagtgaatgccctttaatgacagcctactgcaaacccctctttgcttagtgagaaagccccttgaggggaggtgaattcctgaaagttggtgggaaactggagtacctgccttggtaggaccagaacccatgctagctctgcccctagcagggtgactttggacagggcccttcacctccgagcctcggtttccttatttaggataacaagatagtaatgactaccccccggggccagtggcttgatgtagctgctcatgcacaagtgctgtagatgtaaagattgtggttaggagtggccagcttgggcctggaggctatgatttctgactcctggactggtactttgccacaggcctttccagccctcctcagccccaatccctgaggacagtctgtggctgccccactgggagatgcccagcgttggaattgctgaaggaggcctctccaggaaggcttctccacttgtgtgccctcccatggggccggcctgctcctaccccacccacttccccttctccaggataagcccgctgcaacagctccgtctcccagtgtctcagcctctctggctgctcagcccagcctggtccagctggctatgctggcatggaccctagtctggtggccagttataaatagcccctgcacccacagccttggcagggtgctcctagtgtgggtgcagaggacacttgagaagaggcagccgtgcctgccatcctgaccatgccaggcccagagctgccatggaggcacccaggagcaggcccaaggacacatgggccctatgagctgaggaacctgccctgggcacagggttctgcaggcccaacgtggccggtcagggctggctgtaggcttgaggcatcactttatctcatgactggggatagagcatgggagggtggcgacccagagggcctgtgaggcttggggctggaggcagaacaaagtgggcctgtttgcctgcctgattgcttcctttgagcccaactcattagagggcagctgggcaaatcctctgattccagggagggatgaagggagggagaaggcagggccaagaatgtcctaccataggatctgcagcccatcggagatcactgtagccctcagcccccggcccagggaggagcatgtttagtggaggagcactgtggtggagtcggaagactccgttcaaacaccaccagtgatacagtgttactgtgcacagttcatgtgacttttttctcacgagtaaaatggaattgataatacctaccttgcaggaccacgacaggattaagtgaggaaaaacccccatgagagtgttttgccattgtcaagtgagcctgagggaggctgaggggggatcaggctgtatcatgcccccgaggacaaactttccagtttaccctgctccctctctctgtccctaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgagggtaaagatggagctccctgcatgtggggccaccttgagcttggtccccagcctccccgccttcagcatccctagacaccagtctcagtcctcaactccttgtccctttctgggctgccggccctgcccacagctctccatggacacgccgatgcccgagttgcaggtgggaccccaggaacaagagctgcggcagcttctccacaaggacaagagcaagcgaagtaaggaggtggccaccccagcccagcccagccccaccagccaggtgcctgcggctgcctgtgtggcttgtgaggggtgaggtgggagggccgcagccagctgggcctgagccgaggctttccctctgcttgcctgggctctgcctgtgaatgtgtgggggatgtgggggtggggggtggtgtctggaggctctagggaggggtagggcctcgggcttggctcttgcctagggagtccctgggacgcctcctactgaggatggggacagggcagtggccaggtagtgccacgaggccgcctgtccacccacctccagccccctcctgactctgcccctacaggtgctgtagccagcagcgtggtcaagcagaagctagcggaggtgattctgaaaaaacagcaggcggccctagaaagaacagtccatcccaacagccccggcattccctacaggtaacaccctcctcacctgccctcctgtccccacatgcaccctccacccccgcccccgtgttagccatgagcacacacacttgcctccttcctccccagttgccacaaccagtccttacccttcctctaaacataatgcccccgagcccctctctcaactgatacctgccctcctctctttaaattctccccaccccacctgagcccttctcacacacacacacacacacacacacacacacacacacacaccctactggcttttttcttgcacatgagccccttctctctttatattttccctgagcgcaaaaatgtcctggttcccagagcaggttcctagtgcaggtgacagagcccccagtgggctcttcacccacgcaccaagcacctggcttctatgcctaggtcagagctctgcactatagctgctggagcggacaggcggcccactgctgccacctggagatggtccccattcctaccagccccacattgcccagccctgcccgctgcagcctccccctcaggtcccagggcccgtgctcagggcagtgcccgtgcagtgtccccacacctctgaagcagccttggcactcgttctgtagaaccctggagcccctggagacggaaggagccacccgctccatgctcagcagctttttgcctcctgttcccagcctgcccagtgaccccccagagcacttccctctgcgcaagacaggtgagctgaacaaacaggcggacctttcaggcaaaggaagggggaagggcctcggacatgggggaggagtgtgcaggggtgggcgggctgggctgggccaggagatcatcggtaggctaggatcttctttctagttctgccttctctttccttgaccgatagagatgactgggactgaggggttaaagggtggaaaaaagaagggtggtgggggcctgagtgacgctggtgctccctgcagtctctgagcccaacctgaagctgcgctataagcccaagaagtccctggagcggaggaagaatccactgctccgaaaggagagtgcgccccccagcctccggcggcggcccgcagagaccctcggaggtgagggccggctgagccagtgcggttgccatcctcaaacctggctggtcttgtccctttgggtcaggccccacctagacaggggtcccctccttaggactgccatgcgtggttgcatgggggtgtcccacacacttgccgaagcgggggagttggggctgaaacccagtccctgctctgttagctaaggtgacatctcccagaaaagggggcacccttcaaaaaattatgcaaaggtgctgtaggccaaaggtggcctgtcttcttcctgctctgatgggaagaaagcaggaggccgggcgcggtggttcacgcctataatctcagcactttgggaggctgaggcaggtcaatcacctgaggttaggagtttgagaccagcctggacaatatggtgaaaccctgtctctactaaaaatacaaaaattagccaggcacggtggtgcatgcctgtagtcccagctacttgggaggctgagccaggagaatcgcttgaacccaggaggtggaggttgcagtgagccgggatcgtgccattgcactccagcctgggcaacagagcaagactctgtctcaaaaaaaaaaaaaaagaaagcaggaagcaaaggtgctcgttaaggaagggggtgctcccgggtctcctgcaccaggcggcctggctgtaccctcctgcccaccctggcctctgactgcacttccctctctccccaaccctcagactcctccccaagtagtagcagcacgcccgcatcagggtgcagctcccccaatgacagcgagcacggccccaatcccatcctgggctcggaggtaaggccttgccgagactgggctctcctggggcagttctgaggctcagccttcttccagcaggcggccctacctgggctggggctgcagggtctgggcagcccctgccagagcctcctgggtgttctggggaaggtgcgccggggtagaggtctgggaccggtgaccctcgccctgctccctatggcaggcgctcttgggccagcggctgcggctgcaggagacttctgtggccccgttcgccttgccgacagtgtccttgctgcccgcaatcactctggggctgcccgcccctgccagggtgagtggctggggtgcccacccccactccaagcccccccagcttctttcactcccttttcttgctgcctcaccccatcatcttcattgttctctgctggaatcttcttccccgtgacttctcccgcctctcccccaggctgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactggtgagtctgctgcttcttcagggaaggggctgggtccctgcaccctgctaagagccaagcttgggatggacccatccttcctccccatctctgtccccctgtcctgcgcattgccctgctgcaccctggccccccaccctataccctcgcttccttccattgctccctgtggttccctccttattccaccccccgattctttccagtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaaggtgagaggaattgggcagtggaggtattgagggagtgtttaactggggacttaggggccaaaagaagaggggtacttagaaagggcagggaactggagggcaaaagagggggatgtggcttcttggggcccagagctgcatggcagctggagtcctagcaagatgactggctgcctggcccagcccaccacctcccaccccatgcccctcgctccaccatggcttctcagccaggttctcccctccccagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtatggcagtccccacctgcccctcagaaagtgtcctcagaagactctggggcctggcataagatggggaagggagggagatacgacatcagtgcaacaggcagctctaggacccatggttgccctataagatcctgggtgctggatccacactaaggtgtaggcacacacacacatgcacactcacacacccatacacacacatacacacaacagcttgccagtctcaggtgaagcatgtcccttttccaggaaggagtctgtcctttcctggagccacacatagtcctgccctggtatgtctcccctctatccagaaaggtggctagggccagaggtgggatggagccaggctccagcgtgtccagcagaatgctctcactgtgggaatcaagacccagtgcattagcttgccggagctgttgggatacaggctgggcgccttaaacagcaggcatttaccatctcacagttctggaggctggaagtcaaaatcgaggtgttggtggagttggttccttctgggggctgtgagtgtgagggcaggggctgttcccggtctctctccttggcttgtggatggtcatcttcttcctgtggcttcatgtggtttccctctgtgaatgcctgttcagatttcctcttcctataaggacaatagtcatactgggttaaggcccaccctaacgacctcattttaatttgattacttctgtttgtaaagaccctatctctgaataaggtcatgtttagaggtactgggggttgggacttcaacatataaatttgagggtggggaacataatttactccataacacatgatgacaggccacacacatgttcttgaacagttacatagtccaggacaggaggacatcctggtcagcacaagatccagcgcccctcccctggttcctggctttggagccccaagggcccggggagctggtggaatgggtggtcagtctggggtgcaaacctgtcagggggctgcaggggacagggggcttagagagtcaaaggccagagccccactggacagcaggtctaggtttatcctggaatctctctaggaaaggggcctgcttggcagttcccaagacctcaggcagaagtagagggagcaggaccctgaaacactggagaccaaggccccatctttcccctaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacaggtgagaccgggaggagggtggcgggtggagggaggggctcggctgcacgagtccatgtgggtgtcttggtgtcacttgggacatttttagaggccacagagtgcttagccttgttaggcccacgtagtacccatggagcacatggaacagctggttattgcatttggccgatgaggaccaaggctcacgaaatctaaggtgacaagcctgtggtcaaatagcaagtggtagtcaagccaggataagaattccagtctcctgccaatgcactcagcccttttaaaaaatgatatttattactgttgctctgcttgtaaaaagagctcagaagtattttagacattttgaagagtacagaaaaacctaagaagaaaagaaaaatcaccaaccagtatcccaccacccagaagtaaaccctctgaaattctgctgtatttcagtccaggctttgttctggccatatgtacatgtacagatgccatgaacacatgaactacgttttgtatcctgctttacttagttaacatattatgaatccttcttggcataattagtctttaaaactgagagtctcatagtatttcattaatgtatttcatttttactgagggatcccttatcagttagacatttagattatagcttatttgaatttccttatgcataaatctttcctcatgcttgggattatttcccaaggttcactctaaagaagtagcattcctgggtcacatggtgtgagcattttgagagtcgttggtgtcctagacgcacagaaattgaccctcccacccgcaatgagcaggaccctcctctgaagacttcccacgggtcagactgaaatagtcacttctccaaagctggatttgtctatcacatggtccagtgtcatcgtgggcttcttagctcccggtttgggggtttcctaggaattcttttccctcctgctgccccttggtagcctctctgggttcatgacacgtgtgtgagatgctgtacgttaggcctcatgctttgctctgagcacttgagggcctggtgctgggccctcgagtcctgggccacccagcagtcgtgtccgcatgtgggaaggccgtggctctgtggcagccatgttggctggagctgtgttgatgtgttggctgctgccacctggggaagtgtctgggcgtgtgtggacaggtgtgaggggctggggctggggtaggggcaggatgaaatgcaggcctgtgtgggtgtgaggggtgtgtacccgcccggctccatgtgggtgcctgctgtggcggtgctggcgtgttctctgcagccaaggccatggggcgtgaggactccctgggtccccgctctgacccttgctcctgcagggctgatctatgactcggtcatgctgaagcaccagtgctcctgcggtgacaacagcaggcacccggagcacgccggccgcatccagagcatctggtcccggctgcaggagcgggggctccggagccagtgtgaggtgaggaggcgcgggtggggcccgaggaatgggtggagggaggagtcatgggaggggaggggtggggggccctggggcccatgagagatgaggggcacatggggtggtggtgatgggagggaaggggcgagcatgagctcagagctctgtggtccccttttcagtgtctccgaggccggaaggcctccctggaagagctgcagtcggtccactctgagcggcacgtgctcctctacggcaccaacccgctcagccgcctcaaactggacaacgggaagctggcaggtaatggcctagtggccctgtctccccatgccagcttacctcacccagctcccatgcactcctgtctcggctctgcccgcccagccagcctcctcctgcaccctggacgtccctactccagcttgctgccaagcccctcctcagcccacctccacctctcccttccatatttctctcccccaatactcacccggccctgctctctgggctccctgctccctgttgggccaaaggctgagagtattcacgctgacgtgggctgggcctctccccgcagggctcctggcacagcggatgtttgtgatgctgccctgtggtggggttggggtaagtgtgcccaggggtctcaggggggcgttgccagggctctcagctctcctccctgtggttctcccaggcccagcccctgcagaacctctgcttgttgtggttctgccagacagggtgagccagggacttcctgaggtgccccctgcagcaggaagctccttttggacaggcgtgtctcggacccacagtctcccccgaatgcggagtccagcctaagccttcccctagaaggtgtctggtagatgttgagtgaggtttcaggagcagggccaaggctggggcttaggatcatctctcccttcaaagacccccatgactgggcattggccgccaggctgctctgtctgctcttaagtggcaagttggggtacctcagcctggtccccagaccttgggctgcctggtgtgacatcacggtggtgcttccggtgtccttggcgatcccagcactccccactccgggacatagccccaaactccgctcgcgagctttgcttcctaagtcctcacccctttgtgaagggagcttcccgctccctccggctcagctctcctcgcctaacactatccctgcagtagtttctcaagcaaggtgtgtagaggcaggggatggaggcctcattccggagggaaagtgggagctgtagctggtgggggactttgggagccagtcagtgccctattcacaacttcccatttcttgccactttctggtttttccaactgttgttgcttctgttttctcctccctcctctccctctctgtctttctctctctctctctttctccagcctcttgcgactctctctgccttcctcgcctctcttggtctgcctcgccctccccatctccccatcatgccccccggcccctccctagccttgaggcccagggactgggtttggggggcctcccagcctgggctaggggccctgagtggaagacagtggtgcagacggcccctccagctccgaccgtcccgcagggcctgagcagagtcagctggggcttaaaaccccctcccggcccaaaccccaagtcccgcccaggtaacgccatgccccctcccctgaccggggaggcaggcgtgatgctgccagcagagtgctggccagataatgggctggtgctgggacttaagctgggaaaaagtcatgctgggattgggggacacaggaggccttgcctttgggcggtggggcactggggaggcagcactgtctgcccagctccctgcccctggggtcctggccgtggggtggggaccacccccttgggccctggctcctgtgtgaagccttggatgatgcgggccctgactctggctcccgcaggtggacactgacaccatctggaatgagcttcattcctccaatgcagcccgctgggccgctggcagtgtcactgacctcgccttcaaagtggcttctcgtgagctaaaggtaggaggtttgggttgaaggtggacacaccacaaaggaggaagcagagtggggtagtggggaatccaggcccagaaccccaggcatcgcattcctcttagagattgctacagggttttggaggggaaattgagggctctgggaaccaggttgagattggaactcttggggtacgttcatgcagctgtgggtcagagctgtctgttgattgacaagcattctttctttttccagaatggtttcgctgtggtgcggcccccaggacaccatgcagatcattcaacagccatgtaaggctaagggaagacctgggtgggatgaggtggggggcaagcccccaggaacttccttcagggacattctctcttcttccctgagctttctcaggctgggccaacccaggggcctggggaggtgagggcatgtggagagaatgggctggcaggacctgtctctccttccaggggcttctgcttcttcaactcagtggccatcgcctgccggcagctgcaacagcagagcaaggccagcaagatcctcattgtagactgggtaggtgcctgtccgtagcaccctccaattcgagagccctgggggaaaagccctgagcctgatgttagagatgcggcttcatgtcttagttctgcagtagcctctctgagcctcagtttccccttgtgtaaaatttgggtgaagataacacccacatcacagttgggaggcctagaggggatggcgtgtgggaacgcattcagccatcgcaaacccctgcaacgagtaggagctgtcatttgagtgttgtcttttgacctctattggcttcttttggcagatctagtaatttctgcatttcctgtacaggtagtgatgataagaataatagcagataacatcagtacaccactaatcacatccagacactgatggttttacacatgatggatttaatcctgactataacccactttacagatgaaagtgagcacagagagattaagtaactcacacacagtcattcgtaagtcatgagatggatttgaacccaggcgggttagctctagagtgcttgcgtttaactgctaagctatgtcccttctgcactgacagctgtgtaagagacatttctaagcagaagttgagagcggtggaggacctttgcacacttgagttcccgcatggtcctgtgagtcgagtgtagggcccagctctccctacgagggatggggctgggcctcgtgtacctgcccctctgtaaccgagcttggtttctgatctcctcataacttcatgactttatgcaagacagagtggttcctgatatgtgtaaccctgaacccttccctctccttgccactaaccccatgtccacacagttactctctcaggtgggctggcctgagattgggacacctcctctccttcaggatctcatattacagccagccctgtccagcacagagaggccgaggttcagagccgggcagtggattacgtggggccactcgaccgtgtggctttaggaaccccaggttcctgatcccagtctagggtcctgacctcagaatggccactgaccttgaaacccttctaacctgtcctggcccccatctctctgccttccctaatcgctgcccgtctccctacacaggacgtgcaccatggcaacggcacccagcaaaccttctaccaagaccccagtgtgctctacatctccctgcatcgccatgacgacggcaacttcttcccggggagtggggctgtggatgaggtaaccgcatgtcagggccacatcttccagcctcattgacctcctcctgacacttactctgcctctgtcatgacgagctgtgtgatcctgggcagactgctgagcctctctgatcctaaacttccccacctggaaatggggaggctggatgagctgggctggcagctctaacaaactggtgttccccttctgggactctgctgtcctcatgtctctcttgcctcctgttttccaggtaggggctggcagcggtgagggcttcaatgtcaatgtggcctgggctggaggtctggacccccccatgggggatcctgagtacctggctgctttcaggtacgtgctctgggggcccagaggggcaagtccaccctctcctgtcccttctcccaagagcaccaggggggaggtgatcagttggattgtcagcctgtccccaccagttcctagacattgtaggtgaacgccagtgagaataggacaaacagagagaagaatgcaaaagtcaaagggtgctttgcaaaggcatacattaccgagagccaatgtcaaactgattgctggcaggtgggtggtggagtgagcagagctggcacatttagtcagagaaggctgccactctatttgggaaaagagaattctggaaatggatcttcaaacacttttctggagttatctccatgacagctaattctacgagagccctgggctggagttcctggagtcttctcagagcccagggctatgaagaacacccaagcaggccccagagttggatcaggggtagaggaaggcagctgggggggggcctggaagagggagaggatgagagaatgtagccaggtggcgcagaacctcagaggacctagttgtcccctactcagctctcaagtagtgagtagctgggtgtggtcaactcagtccaaaggactgagcacttgtagtccctgaagctttgtgaccagagtccatctccgcaaggctgtgagattaccctttccctgtggctccggccactgcaccccacagatgcttgcatgcacacacacacatgcacacacacatggtcacacacactcctctttcattccctctggtgctccatccttggctcctttctgcctcttgctagttaagtcccctagggggctgagtccttaggctgtgtgtgccagggacatgggtggccagccaaggtcaaggaggtcagagaaatctgccagttgtgctggggcactgggagccttggagtcctaagaacagggtgccccaccgcaaagttggcaggaccgcccctggcaaccctgcacagtacgatgatcgccacttcttgtgacctcacaggatagtcgtgatgcccatcgcccgagagttctctccagacctagtcctggtgtctgctggatttgatgctgctgagggtcacccggccccactgggtggctaccatgtttctgccaaatgtaagggagcctcagctgagggggacgttagggacagagagccaggcggtgcggggagttgggaggcactcccaagtcagaaagggaaggtggcagtggccagcccagggctttcagcctgaggactggagtatggcagctggtcctgaaattccccgggatctcctagccgagcacagcccaagccctttctcaggaccaggccggttcaccacggagggcttgaccaggtcatacccatggggacttaagtccagtaggcaggaagctcagccgtgggactccccaccacagctgggttgattccaggtgggctggcagctcctccagaggaaggggagagagaagcagcactctcagaatagagggtggccacagggcccagagtacagaaagaagagagggtgtagctcagtgaaaaagacacaggctagagtcaatgacccaagttcaggtcctaccttgctgccacttactagctgtggggcctttcccaaggcccttaacctccctgagccttgagtgaagcatactagtaggttttgtacgacattcaatgtgaaagcactttggaaatagtgattgatacatgtgagtcattctttattagggaggaagcaagcagggaagccacaggggtagagaacagggtcacctctccactcccgcccctcccatttctcccctcccaacctctaggttttggatacatgacgcagcaactgatgaacctggcaggaggcgcagtggtgctggccttggagggtggccatgacctcacagccatctgtgacgcctctgaggcctgtgtggctgctcttctgggtaacagggtgagccgtctccctcccccatccatgcttctgtcaggcaggtaagcccggctctcaggactacccaaggagcaggcagatgggatgggacagggtgggagtggccaagcctgaaacaaggtaggcgaagcggaagcctctgttccaagttaggtccaggcagcatctcctggcctaggtagagtgtgcttgtggctagaaggctggggcccctggggtgggagtgagctgggcctgtgggtccctgagagactggtggctgatgtactgttttctataggtggatcccctttcagaagaaggctggaaacagaaacccaacctcaatgccatccgctctctggaggccgtgatccgggtgcacagtaagtgtggagatgggacactcgctgagctcagactgaaggatcttggtggtaccctgccccaccgtggccagatcctagggcttccggtgccagccaggtgacctgctgttggtctggagtaagattcctgtgagtgacccaggcagcaatggtgagcaccccccagtgaggggttatcctctgagcccccccgatggagccagcagggcctaccagacagtggcccctcaaggtagggactggcctccatctctagcgacagccctagaccagggccaggtcaagagcaacactcaggccttgtttgccaaaaggcctggttcccatccctcccctcagtcctggccacaggcgtctcaggagctctgctggcttgggggctgcgtcttgggataacccccacatttgtaaagtactttaaattttcaacttcaactcaacatctattgagcatcttatgtcaagacccataatctatactagggataaaaatgagtaaaatagattcccatatcaagggctgggtaggggagccgtgatgtctttacataatggtaaagacatggctgattcctcttacggtgggtgccctcacactgagccagattccaggacatggttcctcacagcagcaggcaggaggagcattctcctggcctgggcacccatgcagagcggccgtggtgatggtcagttctcccatgttgtgttcgtcccacctcccaggccctcctttcctgagtgctgggctgagcactggtgggggctgtgtttaagtgggaggcccgagcttggggctctgggaggtcactgtgacacagaccctgtctgcaggcaagcaggctctcctgatgctctcaggagccccgcacctgtggggaatgagtcaaaggtggccttgcagccacaggggatgagagaaaggctgggcacctgctaggactccctcacagccatgttgaacccactctgtgtaccctgtcaggctgtgcgggagtgggagcgaagtgaggagaggggcaggtgggagagccgaccctaagtggaggacaggcccgctcctccggggccctgggcctgagacaccaacctcaatatccggtctaggacgcagtgtggaggggcttgctttctccaaccctttctgacctggcatcttaccccaggtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggtaatgccagacccctggccctgggcccacagcctctccaccgcttcattcctccctgcttgaagaccccgggtccgctatgcagccaccccaaccctcccaggcttcctgaccagggttgagaggaagcttagctaaggcccttgctgcagccctggtgctccagcatcccacccttgtccctccccacaggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatccctgggtctagaatgggaactggagaggaccctgagaggagacgggctgggcggcgacccccacagggctctcgagaacagattctcccctccagtatgggccctggctgtggcccccattcctcaggactgcacagaggaggactggctccggctccgtcgggctcacccttaaccactattcctggctctgcaaaccccagactttgcacacagcctcaggctccacacagaaatgtgaacttggcctcagacaggctggcccttcctaggctctaggggctaggggggagtggggagccaagaggtcccatattcctgagtgcaggggtagtccctctcacctgcttcctcagacgactctggaagcttccctctaccactgggcactgagacgaagctccctgacagccgagactggcagccctccatctggtccgtaccctcgccagaggcccccctacatcaacctcctggcgatgccctggtggagcagatgggtgctctgggagtcctgtgcttcctgatccaatggtgccaaacccttcatctccccaagaagcgcagcatacccctgggacccctcggccactgcccactcggggagccttctctgtttctggggcctcccccaccatagctctgattcccaccccacataggagtagcctgactgagggggaaggggtgggagagaagatacagacatggaggaggggaggctgctctggcaaagtcttcaaggcttttgggggtccaggcctggggtcaagaaggaaaatgtgtgtgagcatgtgtgtgagtgaggcgtgtgtgtgagcgtgtgtgtgagtgaggcgtgtgtgtgtgtctttcctaggacccaccataccctgtgtatgtatgcatgtttttgtaaaaaggaagaaaatggaaaaaaatctgaacaataaatgttttatttgctttaaaagtgcctctgaaagggccccccagaagtgagcagtgcgccgtcaagcgggtgtgcagggcacacagctgcacggcacagtgtgggtctgagtcactgccggaggagggaccagggtggtctgggccgcagcccctttaacaatctgcccttcctgccctgcaggaagaacccattctgaacacaccctggctgtccctggctgccccactttggggtgacaaagaggaggcagccctgggctgagtccacagagaccagccctgatatggcaggagtgggggtggggtgggggagtgggaatacaggagctcagccccagctcaccacggcatccccgaagagttgtgactcagacacacctgggtttaagtcccagctgggagctcaggtaagtcaaatcgtcttctggggcctctgtttatcagtagaaggctccagaagtatacactgttgatggctccttcccgctctgatgcaccatgcctgattaagcatacactgagcctagaagagagaaggggtgaggcacaggcctggacaagctcacccagcctaagcagagaggaaggcgccaggctggcagcacctgcctttgtggttctgagtctgtccggtttttccagcaggaagaggagcacctcattaccttagacctggcccaagctcctggcagcctggcagagtgtgagggaagaggcgctaatccctttcccatctggcctggcctctcgggtgtggacaccaaatcccgcaggggttgctgtagctatgcccgtgggcatccttgccctggctggggtgtgctagagagaggaaagctggaggaggagagctgagctggtggttaccccatgccaggagggccaaggcaagagggcctgcagccccagagatactgactctgtcccctgccctccagggcacaactgaactagcggaatggcttaatcagatagctcgagaactgccactaccactccctccctgcccactcctcccaaagtccacctgttcccgcaagagtcccacctcacaagcaaccaccagaggctgatacaaatggccgctgtatttttgctaaagtgacagtgacacagataaggcaaagagctgaggggcaggacacatcagatgggaagggggagaccgtgcaaaatggcagtctaacagaaaatcatccttgtaccaacagccccttccctcccaagttaggtgagcccttgggccagtgtatgggcagaaaagcagatttgtgtccttcagaagggaaatgtaaaaaggtgaaagctctagttgaagggcagtgagaggggctggagtgggagagaaggtctctcctggccggtggtctgggtgcagcaagggcactctgagaaggcagaatggaaacgcagggctggaggggcatgggtacaggtttgggggctctttccagcctctactatgttgcccccttccccaaagcccttacaggggcagaagcacattccccgtgaccctgagtctggcctcatttgggaagtcttctggggtgtatggatgcctgtgtgtgtgagtgagatgggtggggggccacggctatctggctctagcacactcatgggagaccagctctgggaacaacaggatggggtgctgggatgggggtttaagaggtctctgctagatatttctgaactgacctccccaggtgcccaacctggccttgggaagagagtgcctagggcagcggggatggaaacccttgcctgcagcataggtccaggcctcatggccctacaccttgacctcttgactttgttgccctggccttaagtacaaagattcctcactgcgtgctaagaaaacagatccaggccgggcacggtggctcacacctataatcccagcactttggaaggctgaggcgggtgaatcacctgagatcaggagttcgagaccagcctggccaacatggcaaaaccctgtctctactaaaaacacaaaaatttgccgggcatggtggcagatgcctgtaatcccagctacttgagaggccaaggcaggagaattgcttgaacctgggaggcggaggttgcagtgagctgagatcgcactactgcactccagcctgggtgacagagtaagactccatctcaaaaaaaaaaaaaaaagaaaaaaaaaagaaagaaaagaaaacagattcatttgaaaaggtctaaagctgccctctggccaggctgatgaggagcaacatggcaggatcccctctctaccacactcaggtttcctcgaaaggggacgggcaggacagcttcctgggagaccacactcgtcctgctgtgtattttctgccacagttctggggtcaccagggggtgggagtagcctctcccaacatctcagaggctgagtccaggtcctaaggcccccccagggtgcagagacctcaccccctgggtcagaaatcgctgaggatgctgatgtcagcaaagtcagcccggtagcggtgggcatagaggctgagcaggaaggcccacttgaaggaaatgaagctccagacgctggagaggtagtagctggtggggtctgtgaggcctgcagggagagaagtaccgacagtgactttggcaggccctgccctcggcctctgcaccggccacaaggccagggccaccctccccactcaacatccctgcaacgtcctggcagctgaaccaacccttcagaaagacactgtgatggaggttttagagccatggtccccaacctttttggcaccagggactggttttgtggaagacaatttttccacaaaaaccttgggatggtttggggatgaaactgttccacctcagatcgtcaaacattagatcatcaggggcacgcaaccctcgcatgcgcctcacaacagggtttgcgcttctaatgcccgccgctgacctgacaggaggcagagctcaggcggtaaggctcgcttgcccctcacctgctgtgtggcctggttcctaacaggccacggaccactactgttccacagcctggaacagtagaacggagctggggaccgttttagagtattccagggttttcaggcaggaggccgtggggattagggaagggcagggctggccaatcacaggtctgtatcatccactccgcttttgtgacaaataccctaggccacctccaacctgctttagttagcatcagactcccggaagaaggacctctggttatttccagccacagccaccagagggcgctcccatcccagagctgagcacagactaatgggtcccaatatcaggagtgcttgtatgggctatgcactgctcaagacttcacatcagtttcatagaatccacccaacttgcgaggtggggacttgttttctccacatgaaccataacaaaacccgtgtccggggaggttaagtaattcacccagtctctttggaggtaacctgtggaaccaggatgggaatgctgctaaaatccccgctgctgtccctcccatgccctctttccctgggacccgccctcactctgatgccgggtgatggccagcacgaggaaggtgcagaaggcagcgatggagacggccgagaagaggacgcccacgaagaagaagccgcgcagccccttgagccaggtcctccaataatcttgcatgtacatcacgtgcgtcaccaggacccacagtgccagcacccctgcaggagagacacatcagggcccatccccagggtgctccagagccctgcagatccactagaccaggcctatttgcataagaataccaagaatatggtcttgtctctcattccctcatagaatggagttttccttttcggaggctgtgatatcagaagcaggtaagagactgagctgcctgctagtaagcccaacattagagaaacctgcagaaatacaaaaccctaccattctctctgattttattttgttttggaaaacatgaatatctctcacaaaaatgttatgttaattcatgttatttttagtattactttaaaatgctaaaaaatgttctaacttctaatatagtatatatcaatagctaaaactcacatacataaaatttgcatggagtttcactacttttcaagaggataaaaggatcctgaaaaccagaaagtctgtgcctcagcagcccagcctgctttctggggacttgccactgtctaccctgagggccacagagcagggaagccatgctcctgccccatgctcctcctccacccagccctcagaggccacagtctcaaagtcacaaggcctgcagggctcttcccaaactgcctgagttggttttgcaggatgaggaggtccagccatgaagactccagggccacccaacccctgagtggccttattgttcttggccagacctcatgccactccctccattttgcggcaaagggcaggccatcaccacctgcaccgctccccacccccactgcccccagcttttcctgggcctggcctctgcgcctgggtggacacttcctctaggatgccagctgccacccagccaatacaatacaaaacaaccctctgtgccaggcagtgcctgctggtgcagcccaaagagtggaacaaatatcaactccattggcaagggtccgggcagggcggtggatcccggcaagggacaaatgaggaaggaggcagagccagctgcaggctctcagctggttgggttgcaaggtgtgggccactcaggccgctgcatcctaggagtcaggaggcctttccaaaaggcagctgcacacatgccactcagagtagttaccaaatgtgcacaggaaggataagcatctggttggggaaagtggttatctctggcaatggagacaaggaagggaagttgatggggcggtgggggtggggaagtgctttgaatctatgggaatctatgctattttgtttcttaggctgggtgggaaatctatgctattttatctacgctgttttctcaggctgggttataagggtgtttgttaaatcatctatccttttttgtgtgcctaaaatatttcataatttgtttaagggggaaaaaggcaggaggaagcttgtcactgtggagctatgctctggttaccagctcagaggctgtcccagtttggctgtccccagacagtccagtgaggaacaaaaacccatgagactccaagccaagaaaccaggagcctggcactgctgtgtcccaaagccttggtgtttccacatctaaaaatgggtgggcccaggggtcctgccagtttaggtaactgggcccagggttctaccctagtgagggtgaaggcctaccaagcctacagcttccctcagcccctgccaccgtttgaccccctgaacactctcccagctttgcccctgcccacctccactcttcctcagggggtgggggcctggaatgtgggtccccctcctactgagatcagggtgggccaggacaagcatctgttcctccccacctagtctggatgctggagtgggccagaggtggacaggacaatcatcctgcagcttcctcttacctcctacaagatctggatttttataccagccacctttcacagaagtccctcccactcctcacatgtgtcccatgttttcatttgagggagctgtttggtttataaagggccctgggaacaaggggtgttgtcactgagcacactcaaggagcagtctgatgggcacctgtctggaacaggtcccctcgccctctgtctcctcgtgccacagaggggttggggaagttgagctctaagatcctttcccactcaaagattctagaagccagcttgggagaaaaataaggtcagaacaattcctctgtgactcagcagtttctgggttaggaaatgctgaaataggggcctgtgaagatgccatctcttagtccactggccacgatccagtggttccccaaaactcccacccccatgtacatgcacataccaaagagggctgagggcaggagggagaggaagacagccatttgggggcgttttgggccctgttctttgttaacaacaggcatggtttacccacctctcaccttccctcaaccttgctccaggccctgcacagaggcctcccctccagataaacacacatcaagtgtggaggcaggcctgctgcccacgccctggctcccaccaccctcctgggaagggagctctccaccagccctacccaagggggtcactgaaggagatggagcccctcactggtcccctcccacacatcaggcaggaggcaatgtatctccctaatacccttccctggcacacttggggactctaggaagcctggtttataaaactcggggtggaatggacaggtccccaatcccacctggctgcaagcccacaccagaccacaccgttgctccatcctacctgtttcaaggagcagagcccctgcttagccatgtgagcttattggctcacagccgataagctaagggtaaggaaggcatagggtagggcggtgaaagcatccccagtggctgggctggaggtgaggagacaggagacacccgggggatcccacagggtaaatgcagacaagaaggacactgggcctaggtcaggcccctctattccttcctaggaggaagacagtcctggagccccagggctcttgctcacagctcacctgggaaaaacatagagctgtttagacttcaggagcaacttttctttttctctttccttggtctactaaacccagagcaagagtttgtgttttcccccaggactcccacagcaaagccagaccaggtagatccaacccacacctgcccgcctccccaggaaagccagtcttgcttctgccactgcctttctccaaagccacttcttcgaaggtacccatcaggatgggagagggtatccccacccgtgaacactctgcccaccagacccaggggctccatgactctgctccagaaactggcacatccaatcagtccttgcctggtccaacacaggagaaggagcatgctggggctacccttgggtgactcagcagattctgagctataaaacgctgagtcagaagtagggtgctcacacgttagttaatctgctgacactttacccccaaaagaggaagaaggttgactggccacagaccctctgagaactcactcatggtggggaaggtcggaggcccaggcttggcaacccctctccctgccaccatccaccaccaccactaccaagcagttttggctcttccacagccactgtcaaagatcagaccatcagggagataccaggaccctactccagctggctgctgccctagcgagctgctgccctagcgagctgctgccctagcgcctctctctgaaggcagctccacagctggcctgcctcataggccaagggaagcctgtggggagggagagagggcagagttgggtcggaagaaaaccccaggcaactccctagagtataagaaagaaggccttttccacagccctctaggaggcagctggagactcagggagtgggcagcactgcaagatttcagatctggagggaagtcagatgtcttttggggacatctcctcccactcctaatagatgaggaaactgtggcccaggatagggtcaaacggccaggtagggttcacctccacttgtaccaggccagactcaacacaaccagctgagatcccaggctggccctccctgctcatccttctcttgccttcggtcttcgcaatgcaatcatagggctctgacgccgccttccagaggggaaagcaacagggccaataggaaacaaaaaagtggcagggagaagtgaaccttgacaaacatggagggtggcggggtgcagtggggaaaggaactagggctctgtagataacgcgcagagatgggccagttgtcctcaggatcccacaggtggtgaggcagctgggcaggcagagaacaccctagccagggtgcccatccccccttcttggtgacccagtgctttgttggcattgtgtagggtggggccctgtatgcccctcttctaaggcattaaccccacctcatgctgcgactctacccagggatggcactcagcactccctgtggcagtgagtaaattagttatttttagttattccatttagggttcttttggccgaggattttagtaatggaatataatcatcatatgtaaaagtattatacgtattttcttcccaaagtcctttcccacacatattatttcatgtagctgtgtaatcccgggtaagcaatttatttaacttcaggcctcagtttccttatctgtaaaatggacataatcgtaaggcctacttcatgagactaatgtaaggattaaatgaaataagggaagcaggttgcttaacatagtgcctgacacatagaaagcattttttaaaatgttagaaagtagagataatatttttcatcttcataccaattctatggcgcagagaggttaaactgcgagcccaaatgtgcacaacaaggagcactggtcagcccagagcccccatcagcgcaccgatgcttccccttccctcccagaagcacagaaagggcggctggcctgcaggatttggcagagcttggctgaggagccacagcagacgggtagcaaaaaggcaccaacatgggtggaaagagaatgtccagtttctatgagatccctaacgccgtcctccctgtgggtctcagcagagggctctgcgggggagccctctggaaggagggagatgggcttccccagccacccctagccctcttcctttcccacttcttccctctctgccctcacccagcacccagggaaggggcgcctggggggcaggggatccctcagaacaaccgtgtgtgtgtgcgtgtgtgtgtgtgtgtgtgtttggggataggaggcgaccagcgccgcgggcgcgcggaggggacactgtcgtggggcaactgggcggagggcgacaggcagtgtgccgtgacagataacccgggcggcgcggcaggtctgtgcagccggcagtggcgggccgtcggcacccccaggtggcgcccgggcgggcatcgggtctcagaggcacccaggaatgcccaggagcccgggcccgcgtggggcctgccgccgccaaccgctgttttcgagtaaacactccacccgctccgcggggaagcgccgctcgcggggagcgtcctggagcagctgacggcgcccgccccgcagccgcgcacccgccccgcgccgcccagcgtcccggggtacctgcgagccctcccatggccgcggtccccggctgtcggtagaccaccgtccagacgaggaagatggagaagccggccacggagctgatgccggagtaggcggcgcggaggccgagctgcagcctggacggggccatggggctgcggcgccgggcgggccgagggcgagcgcggccgagagtcacaggtgcagggcccgaagccgccagccggagccgcgggagccgccagagcagccggcgccccgcgctccgcccccggcccgcccccagcgccgcccgcacagcgccctccccggccggaggccgcccgcgcccggaaccgcagacgccaccgccgctttgcgaagctgccgtgtcgtttgggggaatgctgctaaaaaggcggattcctgggcccatccccagacccgccgtgtccggattgttggaccttggaagggaccggaaatatgcctttatacgagcgaaccgcgaggattaaaaatctttacctctgagtacgatgcgtattttcgtttaaaattaaaattaaaatttcttgttaaaaattaataggtaaaacttaagaaatattagagtacgctgtgaatagaattccttccgcccgtccttctatcaaccaggttccctcccaccatatcgcagaggatttctccacacactaaagtctgggaacccctgcgaccatcaccttctgactgcaaacattctatacattctatcctcacgcacagctccccacaaatccctacttttttttttttaaacagagtctcactctgttgcccgggcaggagtacagtggcgccatcatagctccgcatcctccaacacctggattcaagcaatgctctcacctcagccttccaagcagccaggactacaggtgtgtgccaccacgcctggctagtattttttgttatttattttttgtagagatagggtctcgctatattgcccaggctggtccccaactcctggcctcaagctgtcttcccacctcggcctcccaaagcgctggaattacaggcttgagccactgcaacggacctcacctttccttttatcagcacaagccttatcctcccaaacccctcaccctgaccctcgggatcataccattgtaaggaagagacagatctgtagcaggagggcactgagctcccacgtgagcccagcttccggccgggagctggagggaaaatcccagcacaataacaagggctctgccacagcactaaggatccacatggggaggcccatcatatacctgcacaaaacataaccaataccccttgcattagttttttttgtgtgtgtgctgccgtaacaaattaccacaaatttactgatttaaaataacacaaacttattattattttacagttctgtaggtcaggaatccaaataatggtctcactgggctaaaatcaaggggacatcagggttgccttgtttctggaggctctaggggaaaatcagtttccttgactttttttttttttttgagacggagtcttgctctgtcacccaggctagagtgcagtggcgtgatctcagctcactgcaatctctgcctcctgggttcaggtgattctcctgcatcagcctcccgagaagttgggattacacgcttccaccaccacacccagctaatttttgtatttttagtaaagatggggtttcaccatgttggccaggctggtctcgaactcctgacctcaggtgatccgcccacctcagcctcccaaagtgctgggattacaggtgtgagcccaccgtgcccggccgccttttccagcttctagagtgaggctacccacatgccttaactccctcttcctccttttcccttctaaggacccctgtgattgaggggatagtccaggataatctccccatcgcaagatccttaattttatcacatcagcaaaataaatccctttgccgtggaaggtgacatattcataggttctgggatgaggacgtggacatctttgggggccattattatcccacccaccacaccccctctggaacataaacttcatgagcagggacagtgccagttttgttcacatcaacatctaaaagcatgtctggcatataatgaccccttaataaatgtttgttgaataggtgagtgtgagtgaatgaaggtaagaataaaataaaaacaaaagcaattatacacaaggaatctagacagggtgtgcttaactgccaaacaagggatgtaaaccagtgttaagcacatttgcctcactctagaataatctgggagcttctttaaaaataaaaaaataaacccagccccacccctgcctattaaattttgaatcttggagtgtgtattttaatgagaagtgatgtcaaggctgtgagctcagcagcagccctcctttaggagctgccggtggagagtgagtgctggtcctgtggaggagggagcaagccccgtggtgcggagtgcatttccatggatggcttcagagctggccaggatggacagtactccaggcagtgggaaccgcacgtgtgatggcgcagagggaagaaataaagcggccgctttgggaaactgtaagtcgtttgtggctggaatgtcaagtttgaaggtagagtagcgggtttggagattagaaaggttctgaacataaagggccttgcatgctctgttaagaaggctgtccccctccccttggggctgggggagaaaactcacaattctgtgttttagaactatggccagagtaatagagtgaaggcgaactgcctgtgaaacactagaagcagagagatgagttggacaaatttcaccccacagtgctttaattaccaggtctttaaaatggagactgcagtaacacctacttcaaagtgttgtgatgaggagtgcctggaagagtgcctagcacatggtagatactcaataaatgtcaggaagtagaattagtagcagcagaaggctgccatggcaagagaggatgaggggcttcagagctggtccaggcagaagcagagagaatggaagagacgaaactgcttcaagagctatttcagctatctgaacctaaaggtcaggggagaattcattagctgagcagacagaaggaggagagcaaaaatattatgagtggacatattaggagtatgggagagcagtgagcaagcttgctgtgctggaaagtgagattgcgcaagaaaagaaagagaaataattatgtaacagtagtactagaccaggttgtacaaggctaaggccaggcttaaacattttttaaattgtggaaacaatgaagagctattgcagagcattagactcaggtggggtcagaggcctagcttcaccatttgctgtgaccctgggcaagtgcccctaactcacagatgtccaatccaattgactttctgcctggaagaaaatattccatatctgcaccctccataatggtggccactaatcacaggtggctattgaatacttgatatgtgactagtgtgactgaagaactgaatttttaattgtatttaatttaaattaatttaaatttaatgtatttaagtttagtgtatttaatttaaattaatttaaatttaatttaattaatttaagtagctgcacatgactagtggctactgtgttagcacagctagacccaggactcctgtccctccatctgtacacagggaatgatgatgaaacatcacaggcttgttacaaagatcgagatatattgagataatacactcaaagtgctcaacacagtaattcaacaaattattgctgctgctgttgaaattgttattgtttttattgaacagggattgcatgacatacgccaagtcttaggaagattagttagactataatatccagttagatttgatgggggaaaattgtagaggataaagcattcacaaggttatttcagtggtaaggtgtgagagaattaagatcttatccagtgaaagaccttgagaatgggaaagaatggaatgattgttgagccataaagcacatgggtgtgcaccactcatacacatcttctcatatcagcttccttccaaggtattctcagagagtacactcccaacccagcccaggacagacactactacgacccctacaagatgcacagccattctccctgcctgcgccagaaactactagtgctccacaacacacaccaacatttgtgtgtctctttctgggcacagtacctcccaaatttgaactacacttcccagcttccttgcagtcaaacggatgccatgggatcaggttctgaacaatggaatgaaggcagaagcaatgtgcgccatttctaggctgggctcatttaaaaatcttccatacaacctgcattccctcttcccattctgtgacaattttagaggccatatgtaccacataatggaaagaacctaggcttgaatgaatggatggagcagagctacccctgtcccctagaccctcactggactatag

(19) United States
(12) Patent Application Publication
(10) Pub. No.: US 2003/0078216 A1
(43) Pub. Date: Apr. 24, 2003

(54) Inhibition of histone deactylase

(76) Inventors: A. Robert MacLeod, Quebec (CA); Zuomei Li, Quebec (CA); Jeffrey M. Besterman, Quebec (CA)

Correspondence Address: KEOWN & ASSOCIATES, 500 WEST CUMMINGS PARK, SUITE 1200, WOBURN, MA, 01801, US

(21) Appl. No.: 09/563,728

(22) Filed: May 3, 2000

Related U.S. Application Data
(60) Provisional application No. 60/132,287, filed on May 3, 1999.

(51) Int. Cl. A61K 48/00; C12N 9/99; C07H 21/04

(52) U.S. Cl. 514/44; 435/184; 536/23.2; 536/24.5

(57) ABSTRACT

The invention relates to the inhibition of histone deacetylase expression and enzymatic activity and, in particular, to the inhibition of a specific histone deacetylase. The invention also relates to compositions comprising antisense oligonucleotides and methods of using the same to inhibit a histone deacetylase. Also disclosed are methods for identifying a histone deacetylase involved in induction of cell proliferation, and methods for identifying compounds that interact with and reduce the enzymatic activity of such a histone deacetylase.

### RELATED APPLICATIONS

This application claims priority from U.S. provisional application serial No. 60/132,287, filed on May 3, 1999, which is hereby incorporated by reference in its entirety.

### BACKGROUND OF THE INVENTION

1. Field of the Invention

This invention relates to the inhibition of histone deacetylase expression and enzymatic activity.

2. Summary of the Related Art

Deacetylation of the core histones H1-H4 is mediated by a two related families of enzymes called the histone deacetylases. One family of histone deacetylases includes HDAC-1, HDAC-2, and HDAC-3. A second family of histone deacetylases includes HDAC-4 (formerly HDAC-A), HDAC-5 (formerly HDAC-B), HDAC-C, HDAC-D, and HDAC-E. Histone deacetylase activity is thought to modulate the accessibility of transcription factors to enhancer and promoter elements. Indeed, an enrichment of underacetylated histone H4 has been found in transcriptionally silent regions of the genome (Taunton et al., Science 272: 408-411, 1996).

Functional histone deacetylases have been implicated as a requirement in cell cycle progression in both normal and neoplastic cells. Trichostatin A (TCA), an antibiotic isolated from Streptomyces, has been shown to inhibit histone deacetylase activity and arrest cell cycle progression in cells in the G1 and G2 phases (Yoshida et al., J. Biol. Chem. 265: 17174-17179, 1990; Yoshida et al., Exp. Cell Res. 177: 122-131, 1988). Other inhibitors of histone deacetylase activity, including trichostatin C, trapoxin, depudecin, suberoylanilide hydroxamic acid (SAHA), FR901228 (Fujisawa Pharmaceuticals), and butyrate, have been found to similarly inhibit cell cycle progression in cells (Taunton et al., Science 272: 408-411, 1996; Kijima et al., J. Biol. Chem. 268(30):22429-22435, 1993; Kwon et al., Proc. Natl. Acad. Sci. USA 95(7):3356-61, 1998).

The known inhibitors of histone deacetylase are all natural product and are all small molecules that inhibit histone deacetylase activity at the protein level. Moreover, all of the known histone deacetylase inhibitors are non-specific for a particular histone deacetylase enzyme, and more or less inhibit all members of both the histone deacetylase families equally.

Therefore, there remains a need to develop reagents for inhibiting histone deacetylases at a genetic level, as well as for inhibiting expression of a specific histone deacetylase. There is also a need for the development of methods for using these reagents to identify and inhibit a specific histone deacetylase involved in tumorigenesis.

### BRIEF SUMMARY OF THE INVENTION

The invention provides methods and reagents for inhibiting histone deacetylases at a nucleic acid level, as well as for inhibiting expression of a specific histone by inhibiting expression at the nucleic acid level. The invention allows the identification of and specific inhibition of a specific histone deacetylase involved in tumorigenesis.

Accordingly, in a first aspect, the invention provides an antisense oligonucleotide that inhibits the expression of a histone deacetylase. In certain embodiments of this aspect of the invention, the histone deacetylase is HDAC-1, HDAC-2, HDAC-3, HDAC-4, HDAC-5, HDAC-C, HDAC-D, or HDAC-E. In certain other embodiments, the oligonucleotide inhibits more than one histone deacetylase, or the oligonucleotide inhibits all histone deacetylases. Preferably, the oligonucleotide is a chimeric oligonucleotide or a hybrid oligonucleotide.

In certain preferred embodiments of the first aspect of the invention, the oligonucleotide inhibits transcription of a nucleic acid molecule encoding the histone deacetylase. The nucleic acid molecule may be genomic DNA (e.g., a gene), cDNA, or RNA. In other embodiments, the oligonucleotide inhibits translation of the histone deacetylase.

In various embodiments of the first aspect of the invention, the antisense oligonucleotide has at least one internucleotide linkage selected from the group consisting of phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate and sulfone internucleotide linkages. In certain embodiments, the oligonucleotide comprises a ribonucleotide or 2′-O-substituted ribonucleotide region and a deoxyribonucleotide region.

In a second aspect, the invention provides a method for inhibiting a histone deacetylase in a cell comprising contacting the cell with the antisense oligonucleotide of the first aspect of the invention. In certain preferred embodiments of the second aspect of the invention, cell proliferation is inhibited in the contacted cell. In preferred embodiments, the cell is a neoplastic cell which may be in an animal, including a human, and which may be in a neoplastic growth. In certain preferred embodiments, the method of the second aspect of the invention further comprises contacting the cell with a histone deacetylase protein inhibitor that interacts with and reduces the enzymatic activity of the histone deacetylase. Preferably, the histone deacetylase protein inhibitor is operably associated with the antisense oligonucleotide.

In a third aspect, the invention provides a method for inhibiting neoplastic cell growth in an animal comprising administering to an animal having at least one neoplastic cell present in its body a therapeutically effective amount of the antisense oligonucleotide of the first aspect of the invention with a pharmaceutically acceptable carrier for a therapeutically effective period of time.

In certain preferred embodiments of the third aspect of the invention, the method further comprises administering to the animal a therapeutically effective amount of a histone deacetylase protein inhibitor that interacts with and reduces the enzymatic activity of the histone deacetylase with a pharmaceutically acceptable carrier for a therapeutically effective period of time. Preferably, the histone deacetylase protein inhibitor is operably associated with the antisense oligonucleotide.

In a fourth aspect, the invention provides a method for identifying a histone deacetylase that is involved in induction of cell proliferation comprising contacting a cell with an antisense oligonucleotide that inhibits the expression of a histone deacetylase, wherein inhibition of cell proliferation in the contacted cell identifies the histone deacetylase as a histone deacetylase that is involved in induction of cell proliferation. In certain preferred embodiments, the cell is a neoplastic cell, and the induction of cell proliferation is tumorigenesis. In preferred embodiments, the histone deacetylase is HDAC-1, HDAC-2, HDAC-3, HDAC-4, HDAC-5, HDAC-C, HDAC-D, or HDAC-E.

In a fifth aspect, the invention provides a method for identifying a histone deacetylase protein inhibitor that inhibits a histone deacetylase that is involved in induction of cell proliferation comprising contacting a histone deacetylase identified by the method of the fourth aspect of the invention with a candidate compound and measuring the enzymatic activity of the contacted histone deacetylase, wherein a reduction in the enzymatic activity of the contacted histone deacetylase identifies the candidate compound as a histone deacetylase protein inhibitor that inhibits a histone deacetylase that is involved in induction of cell proliferation. In certain preferred embodiments, the histone deacetylase protein inhibitor interacts with and reduces the enzymatic activity of fewer than all histone deacetylases.

In a sixth aspect, the invention provides a method for identifying a histone deacetylase that is involved in induction of cell differentiation comprising contacting a cell with an antisense oligonucleotide that inhibits the expression of a histone deacetylase, wherein induction of differentiation in the contacted cell identifies the histone deacetylase as a histone deacetylase that is involved in induction of cell differentiation. In certain preferred embodiments, the cell is a neoplastic cell. In preferred embodiments, the histone deacetylase is HDAC-1, HDAC-2, HDAC-3, HDAC-4, HDAC-5, HDAC-C, HDAC-D, or HDAC-E.

In a seventh aspect, the invention provides a method for identifying a histone deacetylase protein inhibitor that inhibits a histone deacetylase that is involved in induction of cell differentiation comprising contacting a histone deacetylase identified by the method of the sixth aspect of the invention with a candidate compound and measuring the enzymatic activity of the contacted histone deacetylase, wherein a reduction in the enzymatic activity of the contacted histone deacetylase identifies the candidate compound as a histone deacetylase protein inhibitor that inhibits a histone deacetylase that is involved in induction of cell differentiation. In certain preferred embodiments, the histone deacetylase protein inhibitor interacts with and reduces the enzymatic activity of fewer than all histone deacetylases.

In an eighth aspect, the invention provides a histone deacetylase protein inhibitor identified by the method of the fifth or the seventh aspects of the invention. Preferably, the histone deacetylase protein inhibitor is substantially pure.

In a ninth aspect, the invention provides a method for inhibiting cell proliferation in a cell comprising contacting a cell with at least two of the reagents selected from the group consisting of an antisense oligonucleotide that inhibits a histone deacetylase, a histone deacetylase protein inhibitor, an antisense oligonucleotide that inhibits a DNA methyltransferase, and a DNA methyltransferase protein inhibitor. In one embodiment, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional preferred embodiments, the reagents selected from the group are operably associated.

According to the invention, reagents found to specifically inhibit a histone deacetylase involved in neoplasia may be used as therapeutic agents to inhibit neoplastic cell growth in patients suffering from neoplasia. For example, an antisense oligonucleotide that inhibits the expression of a histone deacetylase may be administered with a pharmaceutically-acceptable carrier (e.g., physiological sterile saline solution) via any route of administration to a patient suffering from neoplasia or hyperplasia in an attempt to alleviate any resulting disease symptom (e.g., death). Likewise, an antisense oligonucleotide that inhibits the expression of a histone deacetylase may be incorporated into a gene therapy expression vector (e.g., a replication-deficient adenoviral vector), and phage particles carrying such vectors may be delivered with a pharmaceutically-acceptable carrier directly to the cells of the neoplastic or hyperplastic growth. Pharmaceutically-acceptable carriers and their formulations are well-known and generally described in, for example, Remington's Pharmaceutical Sciences (18th Edition, ed. A. Gennaro, Mack Publishing Co., Easton, Pa., 1990).

### BRIEF DESCRIPTION OF THE DRAWINGS

FIG. 1 is a graphic representation of a Northern blotting analysis showing the dose-dependent abilities of representative, nonlimiting, synthetic oligonucleotides according to the invention that specifically bind to either HDAC-1-encoding nucleic acid or both HDAC-1- and HDAC-2-encoding nucleic acids to inhibit expression of HDAC-1 mRNA or both HDAC-1 MRNA and HDAC-2 mRNA, respectively.

FIG. 2 is a graphic representation of a Northern blotting analysis showing the dose-dependent abilities of representative, nonlimiting, synthetic oligonucleotides according to the invention that specifically bind to HDAC-2-encoding nucleic acid to inhibit expression of HDAC-2 MRNA.

FIG. 3 is a graphic representation of a Western blotting analysis showing the abilities of representative, nonlimiting, synthetic oligonucleotides according to the invention that specifically bind to HDAC-2-encoding nucleic acid to specifically inhibit expression of HDAC-2 protein.

FIG. 4 is a graphic representation of a Western blotting analysis showing the abilities of representative, nonlimiting, synthetic oligonucleotides according to the invention that specifically bind to either HDAC-1-encoding nucleic acid or both HDAC-1- and HDAC-2-encoding nucleic acid to inhibit expression of HDAC-1 protein or both HDAC-1 protein and HDAC-2 protein, respectively. Mismatched synthetic oligonucleotides were used as negative controls. Equal loading of all lanes is evidenced by the equivalent expression of actin.

### DETAILED DESCRIPTION OF THE PREFERRED EMBODIMENTS

The invention provides methods and reagents for inhibiting a histone deacetylase at a nucleic acid level, as well as for inhibiting a specific histone deacetylase at the nucleic acid level. The reagents described herein that inhibit histone deacetylase at the nucleic acid level (i.e., inhibiting transcription and translation) allows the identification of a specific histone deacetylase which is involved in neoplasia. Moreover, therapeutical compositions for treating and/or alleviating the symptoms of neoplasia may be developed using the reagents of the invention that specifically inhibit a particular histone deacetylase involved in neoplasia.

The reagents according to the invention are useful as analytical tools and as therapeutic tools, including as gene therapy tools. The invention also provides methods and compositions which may be manipulated and fine-tuned to fit the condition(s) to be treated while producing fewer side effects. The patent and scientific literature referred to herein establishes knowledge that is available to those with skill in the art. The issued patents, applications, and references, including GenBank database sequences, that are cited herein are hereby incorporated by reference to the same extent as if each was specifically and individually indicated to be incorporated by reference.

In a first aspect, the invention provides an antisense oligonucleotide that inhibits the expression of a histone deacetylase. In certain embodiments of this aspect of the invention, the histone deacetylase is HDAC-1, HDAC-2, HDAC-3, HDAC-4, HDAC-5, HDAC-C, HDAC-D, or HDAC-E. In certain embodiments, the oligonucleotide inhibits more than one histone deacetylase, or the oligonucleotide inhibits all histone deacetylases.

The antisense oligonucleotides according to the invention are complementary to a region of RNA or double-stranded DNA that encodes a histone deacetylase. For purposes of the invention, the term “oligonucleotide” includes polymers of two or more deoxyribonucleosides, ribonucleosides, or 2′-O-substituted ribonucleoside residues, or any combination thereof. Preferably, such oligonucleotides have from about 8 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages. In certain preferred embodiments, these internucleoside linkages may be phosphodiester, phosphotriester, phosphorothioate, or phosphoramidate linkages, or combinations thereof. The term oligonucleotide also encompasses such polymers having chemically modified bases or sugars and/or having additional substituents, including without limitation lipophilic groups, intercalating agents, diamines, and adamantane. For purposes of the invention the term “2′-O-substituted” means substitution of the 2′ position of the pentose moiety with an -O-lower alkyl group containing 1-6 saturated or unsaturated carbon atoms, or with an -O-aryl or allyl group having 2-6 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups; or such 2′ substitution may be with a hydroxy group (to produce a ribonucleoside), an amino or a halo group, but not with a 2′-H group.

For purposes of the invention, the term “complementary” means having the ability to hybridize to a genomic region, a gene, or an RNA transcript thereof under physiological conditions. Such hybridization is ordinarily the result of base-specific hydrogen bonding between complementary strands, preferably to form Watson-Crick or Hoogsteen base pairs, although other modes of hydrogen bonding, as well as base stacking can lead to hybridization. As a practical matter, such hybridization can be inferred from the observation of specific gene expression inhibition, which may be at the level of transcription or translation (or both).

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region (see e.g., Pederson et al. U.S. Pat. Nos. 5,635,377 and 5,366,878). Preferably, such chimeric oligonucleotides contain at least three consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide will contain at least three consecutive deoxyribonucleosides and will also contain ribonucleosides, 2′-O-substituted ribonucleosides, or combinations thereof (see e.g., Metelev and Agrawal, U.S. Pat. No. 5,652,355).

The exact nucleotide sequence and chemical structure of an antisense oligonucleotide utilized in the invention can be varied, so long as the oligonucleotide retains its ability to inhibit expression of a histone deacetylase. This is readily determined by testing whether the particular antisense oligonucleotide is active by quantitating the amount of mRNA encoding a histone deacetylase, quantitating the amount of histone deacetylase protein, quantitating the histone deacetylase enzymatic activity, or quantitating the ability of histone deacetylase to inhibit cell growth in a an in vitro or in vivo cell growth assay, all of which are described in detail in this specification.

Antisense oligonucleotides utilized in the invention may conveniently be synthesized on a suitable solid support using well-known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T., Methods in Molec. Biol. 20: 465-496, 1993).

Antisense oligonucleotides according to the invention are useful for a variety of purposes. For example, they can be used as “probes” of the physiological function of histone deacetylase by being used to inhibit the activity of histone deacetylase in an experimental cell culture or animal system and to evaluate the effect of inhibiting such histone deacetylase activity. This is accomplished by administering to a cell or an animal an antisense oligonucleotide that inhibits histone deacetylase expression according to the invention and observing any phenotypic effects. In this use, the antisense oligonucleotides according to the invention is preferable to traditional “gene knockout” approaches because it is easier to use, and can be used to inhibit histone deacetylase activity at selected stages of development or differentiation. Thus, the method according to the invention can serve as a probe to test the role of histone deacetylation in various stages of development.

Preferred antisense oligonucleotides of the invention inhibit either the transcription of a nucleic acid molecule encoding the histone deacetylase, or the translation of a nucleic acid molecule encoding the histone deacetylase. Histone deacetylase-encoding nucleic acids may be RNA or double stranded DNA regions and include, without limitation, intronic sequences, untranslated 5′ and 3′ regions, intron-exon boundaries as well as coding sequences from a histone deacetylase family member gene. For human sequences, see e.g., Yang et al., Proc. Natl. Acad. Sci. USA 93(23): 12845-12850, 1996; Furukawa et al., Cytogenet. Cell Genet. 73(1-2): 130-133, 1996; Yang et al., J. Biol. Chem. 272(44): 28001-28007, 1997; Betz et al., Genomics 52(2): 245-246, 1998; Taunton et al., Science 272(5260): 408-411, 1996; and Dangond et al., Biochem. Biophys. Res. Commun. 242(3): 648-652, 1998).

Particularly preferred non-limiting examples of antisense oligonucleotides of the invention are complementary to regions of RNA or double-stranded DNA encoding a histone deacetylase (e.g., HDAC-1, HDAC-2, HDAC-3, HDAC-4, HDAC-5, HDAC-C, HDAC-D, or HDAC-E). The antisense olignouncleotides according to the invention are complementary to regions of RNA or double-stranded DNA that encode HDAC-1, HDAC-2, HDAC-3, HDAC-4, HDAC-5, HDAC-C, HDAC-D, and/or HDAC-E. The sequence of human HDAC-1 can be found in GenBank Accession No. U50079 (amino acid sequence in SEQ ID NO:24; nucleic acid sequence in SEQ ID NO:25. The sequence of human HDAC-2 can be found in GenBank Accession No. U31814 (amino acid sequence in SEQ ID NO: 26; nucleic acid sequence in SEQ ID NO: 27). The sequence of human HDAC-3 can be found in GenBank Accession No. U75697 (amino acid sequence in SEQ ID NO: 28; nucleic acid sequence in SEQ ID NO: 29). The sequence of human HDAC-4 (formerly human HDAC-A) in GenBank Accession No. AB006626 (amino acid sequence in SEQ ID NO: 30; nucleic acid sequence in SEQ ID NO: 31). The sequence of human HDAC-5 (formerly human HDAC-B) can be found in GenBank Accession No. AB011172 (amino acid sequence in SEQ ID NO: 32; nucleic acid sequence in SEQ ID NO: 33). The sequence of human HDAC-C can be found in GenBank Accession No. AC004994 (amino acid sequence in SEQ ID NO: 34; nucleic acid sequence in SEQ ID NO: 35). The sequence of human HDAC-D can be found in GenBank Accession No. AC004466 (nucleic acid sequence in SEQ ID NO: 36).

The sequences encoding histone deacetylases from many non-human animal species are also known (see, for example, GenBank Accession Numbers AF006603, AF006602,and AF074882 for murine histone deacetylases). Accordingly, the antisense oligonucleotides of the invention may also be complementary to regions of RNA or double-stranded DNA that encode histone deacetylases from non-human animals. Particularly, preferred oligonucleotides have nucleotide sequences of from about 13 to about 35 nucleotides which include the nucleotide sequences shown below as SEQ ID NOs: 1-18. Yet additional particularly preferred oligonucleotides have nucleotide sequences of from about 15 to about 26 nucleotides of the nucleotide sequences shown below. Most preferably, the oligonucleotides shown below have phosphorothioate backbones, are 20-26 nucleotides in length, and are modified such that the terminal four nucleotides at the 5′ end of the oligonucleotide and the terminal four nucleotides at the 3′ end of the oligonucleotide each have 2′-O-methyl groups attached to their sugar residues.

Antisense oligonucleotide specific for human HDAC-1 (MG2608):

5′-GAA ACG TGA GGG ACT CAG CA-3′ (SEQ ID NO: 1).

Antisense oligonucleotide specific for both human HDAC-1 and human HDAC-2 (MG2610) is a 25/25/25/25 mixture of four oligonucleotides:

(SEQ ID NO:2)
5′-CAG CAA ATT ATG GGT CAT GCG GAT TC-3′;
(SEQ ID NO:3)
5′-CAG CAA GTT ATG AGT CAT GCG GAT TC-3′;
(SEQ ID NO:4)
5′-CAG CAA ATT ATG AGT CAT GCG GAT TC-3′; and
(SEQ ID NO:5)
5′-CAG CAA GTT ATG GGT CAT GCG GAT TC-3′.

Antisense oligonucleotide specific for human HDAC-2:

(MG2628; SEQ ID NO:6)
5′-TGC TGC TGC TGC TGC TGC CG-3′;
(MG2633; SEQ ID NO:7)
5′-CCT CCT GCT GCT GCT GCT GC-3′;
(MG2635; SEQ ID NO:8)
5′-GGT TCC TTT GGT ATC TGT TT-3′; and
(MG2636; SEQ ID NO:9)
5′-CTC CTT GAC TGT ACG CCA TG-3′.

The antisense oligonucleotides according to the invention may optionally be formulated with any of the well known pharmaceutically acceptable carriers or diluents (see preparation of pharmaceutically acceptable formulations in, e.g., Remington's Pharmaceutical Sciences, 18th Edition, ed. A. Gennaro, Mack Publishing Co., Easton, Pa., 1990).

In a second aspect, the invention provides a method for inhibiting a histone deacetylase in a cell comprising contacting the cell with the antisense oligonucleotide that inhibits the expression of a histone deacetylase. Preferably, cell proliferation is inhibited in the contacted cell. Thus, the antisense oligonucleotides according to the invention are useful in therapeutic approaches to human diseases including benign and malignant neoplasms by inhibiting cell proliferation in cells contacted with the antisense oligonucleotides. The phrase “inhibiting cell proliferation” is used to denote an ability of a histone deacetylase antisense oligonucleotide or a histone deacetylase protein inhibitor (or combination thereof) to retard the growth of cells contacted with the oligonucleotide or protein inhibitor, as compared to cells not contacted. Such an assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth with calipers, and comparing the size of the growth of contacted cells with non-contacted cells. Preferably, the term includes a retardation of cell proliferation that is at least 50% of non-contacted cells. More preferably, the term includes a retardation of cell proliferation that is 100% of non-contacted cells (i.e., the contacted cells do not increase in number or size). Most preferably, the term includes a reduction in the number or size of contacted cells, as compared to non-contacted cells. Thus, a histone deacetylase antisense oligonucleotide or a histone deacetylase protein inhibitor that inhibits cell proliferation in a contacted cell may induce the contacted cell to undergo growth retardation, to undergo growth arrest, to undergo programmed cell death (i.e., to apoptose), or to undergo necrotic cell death.

Conversely, the phrase “inducing cell proliferation” is used to denote the requirement of the presence or enzymatic activity of a histone deacetylase for cell proliferation in a normal (i.e., non-neoplastic) cell. Hence, over-expression of a histone deacetylase that induces cell proliferation may or may not lead to increased cell proliferation; however, inhibition of a histone deacetylase that induces cell proliferation will lead to inhibition of cell proliferation.

The phrase “inducing cell differentiation” is used to denote the ability of a histone deacetylase antisense oligonucleotide or histone deacetylase protein inhibitor (or combination thereof) to induce differentiation in a contacted cell as compared to a cell that is not contacted. Thus, a neoplastic cell, when contacted with a histone deacetylase antisense oligonucleotide or histone deacetylase protein inhibitor (or both) of the invention, may be induced to differentiate, resulting in the production of a daughter cell that is phylogenetically more advanced than the contacted cell.

The cell proliferation inhibiting ability of the antisense oligonucleotides according to the invention allows the synchronization of a population of a-synchronously growing cells. For example, the antisense oligonucleotides of the invention may be used to arrest a population of non-neoplastic cells grown in vitro in the G1 or G2 phase of the cell cycle. Such synchronization allows, for example, the identification of gene and/or gene products expressed during the G1 or G2 phase of the cell cycle. Such a synchronization of cultured cells may also be useful for testing the efficacy of a new transfection protocol, where transfection efficiency varies and is dependent upon the particular cell cycle phase of the cell to be transfected. Use of the antisense oligonucleotides of the invention allows the synchronization of a population of cells, thereby aiding detection of enhanced transfection efficiency.

The anti-neoplstic utility of the antisense oligonucleotides according to the invention is described in detail elsewhere in this specification.

In yet other preferred embodiments, the cell contacted with a histone deacetylase antisense oligonucleotide is also contacted with a histone deacetylase protein inhibitor.

As used herein, the term “histone deacetylase protein inhibitor” denotes an active moiety capable of interacting with a histone deacetylase at the protein level and reducing the activity of that histone deacetylase. Histone deacetylase protein inhibitors include, without limitation, trichostatin A, trichostatin B, trichostatin C, depudecin, trapoxin, butyrate, suberoylanilide hydroxamic acid (SAHA), FR901228 (Fujisawa Pharmaceuticals), and acetyldinaline (el-Beltagi et al., Cancer Res. 53(13):3008-3014, 1993). A histone deacetylase protein inhibitor is a molecule that reduces the activity of a histone deacetylase to a greater extent than it reduces the activity of any unrelated protein. In a preferred embodiment, such reduction of the activity of a histone deacetylase is at least 5-fold, more preferably at least 10-fold, most preferably at least 50-fold. In another embodiment, the activity of a histone deacetylase is reduced 100-fold. Preferably, a histone deacetylase protein inhibitor interacts with and reduces the activity of fewer than all histone deacetylases. By “all histone deacetylases” is meant all of the members of both of the histone deacetylase families of proteins from a particular species of animal and includes, without limitation, HDAC-1, HDAC-2, HDAC-3, HDAC-4, HDAC-5, HDAC-C, HDAC-D, or HDAC-E, all of which are considered “related proteins,” as used herein. For example, a preferred histone deacetylase protein inhibitor interacts with and inhibits HDAC-1 and HDAC-2, but does not interact with and inhibit HDAC-3. Most preferably, a histone deacetylase protein inhibitor interacts with and reduces the activity of one histone deacetylase (e.g., HDAC-2), but does not interact with or reduce the activities of the other histone deacetylases (e.g., HDAC-1 and HDAC-3). As discussed below, a preferred histone deacetylase protein inhibitor is one that interacts with and reduces the enzymatic activity of a histone deacetylase that is involved in tumorigenesis.

Preferably, the histone deacetylase protein inhibitor is operably associated with the antisense oligonucleotide. As mentioned above, the antisense oligonucleotides according to the invention may optionally be formulated well known pharmaceutically acceptable carriers or diluents. This formulation may further contain one or more one or more additional histone deacetylase antisense oligonucleotide(s), and/or one or more histone deacetylase protein inhibitor(s), or it may contain any other pharmacologically active agent.

In a particularly preferred embodiment of the invention, the antisense oligonucleotide is in operable association with a histone deacetylase protein inhibitor. The term “operable association” includes any association between the antisense oligonucleotide and the histone deacetylase protein inhibitor which allows an antisense oligonucleotide to inhibit histone deacetylase-encoding nucleic acid expression and allows the histone deacetylase protein inhibitor to inhibit histone deacetylase enzymic activity. One or more antisense oligonucleotide of the invention may be operably associated with one or more histone deacetylase protein inhibitor. Preferably, an antisense oligonucleotide of the invention that targets one particular histone deacetylase (e.g., HDAC-2) is operably associated with a histone deacetylase protein inhibitor which targets the same histone deacetylase. A preferred operable association is a hydrolyzable. Preferably, the hydrolyzable association is a covalent linkage between the antisense oligonucleotide and the histone deacetylase protein inhibitor. Preferably, such covalent linkage is hydrolyzable by esterases and/or amidases. Examples of such hydrolyzable associations are well known in the art. Phosphate esters are particularly preferred.

In certain preferred embodiments, the covalent linkage may be directly between the antisense oligonucleotide and the histone deacetylase protein inhibitor so as to integrate the histone deacetylase protein inhibitor into the backbone. Alternatively, the covalent linkage may be through an extended structure and may be formed by covalently linking the antisense oligonucleotide to the histone deacetylase protein inhibitor through coupling of both the antisense oligonucleotide and the histone deacetylase protein inhibitor to a carrier molecule such as a carbohydrate, a peptide or a lipid or a glycolipid. Other preferred operable associations include lipophilic association, such as formation of a liposome containing an antisense oligonucleotide and the histone deacetylase protein inhibitor covalently linked to a lipophilic molecule and thus associated with the liposome. Such lipophilic molecules include without limitation phosphotidylcholine, cholesterol, phosphatidylethanolamine, and synthetic neoglycolipids, such as syalyllacNAc-HDPE. In certain preferred embodiments, the operable association may not be a physical association, but simply a simultaneous existence in the body, for example, when the antisense oligonucleotide is associated with one liposome and the protein effector is associated with another liposome.

In a third aspect, the invention provides a method for inhibiting neoplastic cell proliferation in an animal comprising administering to an animal having at least one neoplastic cell present in its body a therapeutically effective amount of the antisense oligonucleotide of the first aspect of the invention with a pharmaceutically acceptable carrier for a therapeutically effective period of time. Preferably, the animal is a mammal, particularly a domesticated mammal. Most preferably, the animal is a human.

The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastases in vivo and that may recur after attempted removal. The term “tumorigenesis” is used to denote the induction of cell proliferation that leads to the development of a neoplastic growth.

The terms “therapeutically effective amount” and “therapeutically effective period of time” are used to denote known treatments at dosages and for periods of time effective to reduce neoplastic cell growth. Preferably, such administration should be parenteral, oral, sublingual, transdermal, topical, intranasal, or intrarectal. When administered systemically the therapeutic composition is preferably administered at a sufficient dosage to attain a blood level of antisense oligonucleotide from about 0.1 μM to about 10 μM. For localized administration, much lower concentrations than this may be effective, and much higher concentrations may be tolerated. One of skill in the art will appreciate that such therapeutic effect resulting in a lower effective concentration of the histone deacetylase inhibitor may vary considerably depending on the tissue, organ, or the particular animal or patient to be treated according to the invention.

In a preferred embodiment, the therapeutic composition of the invention is administered systemically at a sufficient dosage to attain a blood level of antisense oligonucleotide from about 0.01 μM to about 20 μM. In a particularly preferred embodiment, the therapeutic composition is administered at a sufficient dosage to attain a blood level of antisense oligonucleotide from about 0.05 μM to about 15 μM. In a more preferred embodiment, the blood level of antisense oligonucleotide is from about 0.1 μM to about 10 μM.

For localized administration, much lower concentrations than this may be therapeutically effective. Preferably, a total dosage of antisense oligonucleotide will range from about 0.1 mg to about 200 mg oligonucleotide per kg body weight per day. In a more preferred embodiment, a total dosage of antisense oligonucleotide will range from about 1 mg to about 20 mg oligonucleotide per kg body weight per day. In a most preferred embodiment, a total dosage of antisense oligonucleotide will range from about 2 mg to about 10 mg oligonucleotide per kg body weight per day. In a particularly preferred embodiment, the therapeutically effective amount of a histone deacetylase antisense oligonucleotide is about 0.5 mg oligonucleotide per kg body weight per day.

In certain preferred embodiments of the third aspect of the invention, the method further comprises administering to the animal a therapeutically effective amount of a histone deacetylase protein inhibitor with a pharmaceutically acceptable carrier for a therapeutically effective period of time. Preferably, the histone deacetylase protein inhibitor is operably associated with the antisense oligonucleotide. Methods for the operable association of a histone deacetylase protein inhibitor with a histone deacetylase antisense oligonucleotide are described above.

The histone deacetylase protein inhibitor-containing therapeutic composition of the invention is administered systemically at a sufficient dosage to attain a blood level histone deacetylase protein inhibitor from about 0.01 μM to about 10 μM. In a particularly preferred embodiment, the therapeutic composition is administered at a sufficient dosage to attain a blood level of histone deacetylase protein inhibitor from about 0.05 μM to about 10 μM. In a more preferred embodiment, the blood level of histone deacetylase protein inhibitor is from about 0.1 μM to about 7 μM. For localized administration, much lower concentrations than this may be effective. Preferably, a total dosage of histone deacetylase protein inhibitor will range from about 0.01 mg to about 5 mg protein effector per kg body weight per day. In a more preferred embodiment, a total dosage of histone deacetylase protein inhibitor will range from about 0.1 mg to about 4 mg protein effector per kg body weight per day. In a most preferred embodiment, a total dosage of histone deacetylase protein inhibitor will range from about 0.1 mg to about 1 mg protein effector per kg body weight per day. In a particularly preferred embodiment, the therapeutically effective synergistic amount of histone deacetylase protein inhibitor (when administered with an antisense oligonucleotide) is 0.1 mg per kg body weight per day.

This aspect of the invention results in an improved inhibitory effect, thereby reducing the therapeutically effective concentrations of either or both of the nucleic acid level inhibitor (i.e., antisense oligonucleotide) and the protein level inhibitor (i.e., histone deacetylase protein inhibitor) required to obtain a given inhibitory effect as compared to those necessary when either is used individually.

Furthermore, one of skill will appreciate that the therapeutically effective synergistic amount of either the antisense oligonucleotide or the histone deacetylase inhibitor may be lowered or increased by fine tuning and altering the amount of the other component. The invention therefore provides a method to tailor the administration/treatment to the particular exigencies specific to a given animal species or particular patient. Therapeutically effective ranges may be easily determined for example empirically by starting at relatively low amounts and by step-wise increments with concurrent evaluation of inhibition.

In a fourth aspect, the invention provides a method for investigating the role of a particular histone deacetylase in cellular proliferation, including the proliferation of neoplastic cells. In this method, the cell type of interest is contacted with an amount of an antisense oligonucleotide that inhibits the expression of a histone deacetylase, as described for the first aspect according to the invention, resulting in inhibition of expression of the histone deacetylase in the cell. If the contacted cell with inhibited expression of the histone deacetylase also shows an inhibition in cell proliferation, then the histone deacetylase is involved in the induction of cell proliferation. In this scenario, if the contacted cell is a neoplastic cell, and the contacted neoplastic cell shows an inhibition of cell proliferation, then the histone deacetylase whose expression was inhibited is a histone deacetylase that is involved in tumorigenesis. Preferably, the histone deacetylase is HDAC-1, HDAC-2, HDAC-3, HDAC-4, HDAC-5, HDAC-C, HDAC-D, or HDAC-E.

Thus, by identifying a particular histone deacetylase that is involved in the induction of cell proliferation, only that particular histone deacetylase need be targeted with an antisense oligonucleotide to inhibit cell proliferation or induce differentiation. Consequently, a lower therapeutically effective dose of antisense oligonucleotide may be able to effectively inhibit cell proliferation. Moreover, undesirable side effects of inhibiting all histone deacetylases may be avoided by specifically inhibiting the one (or more) histone deacetylase(s) involved in inducing cell proliferation.

Once such a histone deacetylase involved in inducing cell proliferation is identified using the antisense oligonucleotides of the first aspect of the invention, then histone deacetylase protein inhibitors may be generated that specifically inhibit the histone deacetylase involved in inducing cell proliferation, while not inhibiting other histone deacetylases not involved in inducing cell proliferation. Accordingly, in a fifth aspect, the invention provides a method for identifying a histone deacetylase protein inhibitor that inhibits a histone deacetylase that is involved in the induction of cell proliferation. This method comprises contacting a histone deacetylase identified as being involved in inducing cell proliferation with a candidate compound and measuring the enzymatic activity of the contacted histone deacetylase. A reduction in the enzymatic activity of the contacted histone deacetylase identifies the candidate compound as a histone deacetylase protein inhibitor that inhibits a histone deacetylase that is involved in induction of cell proliferation.

Measurement of the enzymatic activity of a histone deacetylase can be achieved using known methodologies. For example, Yoshida et al. (J. Biol. Chem. 265:17174-17179, 1990) describe the assessment of histone deacetylase enzymatic activity by the detection of acetylated histones in trichostatin A treated cells. Taunton et al. (Science 272: 408-411, 1996) similarly describes methods to measure histone deacetylase enzymatic activity using endogenous and recombinant HDAC-1. Both Yoshida et al. (J. Biol. Chem. 265:17174-17179, 1990) and Taunton et al. (Science 272: 408-411, 1996) are hereby incorporated by reference.

Preferably, the histone deacetylase protein inhibitor that inhibits a histone deacetylase that is involved in induction of cell proliferation is a histone deacetylase protein inhibitor that interacts with and reduces the enzymatic activity of fewer than all histone deacetylases.

In a sixth aspect, the invention provides a method for identifying a histone deacetylase that is involved in induction of cell differentiation comprising contacting a cell with an antisense oligonucleotide that inhibits the expression of a histone deacetylase, wherein induction of differentiation in the contacted cell identifies the histone deacetylase as a histone deacetylase that is involved in induction of cell differentiation. Preferably, the cell is a neoplastic cell. In preferred embodiments, the histone deacetylase is HDAC-1, HDAC-2, HDAC-3, HDAC-4, HDAC-5, HDAC-C, HDAC-D, or HDAC-E.

In a seventh aspect, the invention provides a method for identifying a histone deacetylase protein inhibitor that inhibits a histone deacetylase that is involved in induction of cell differentiation comprising contacting a histone deacetylase identified by the method of the sixth aspect of the invention with a candidate compound and measuring the enzymatic activity of the contacted histone deacetylase, wherein a reduction in the enzymatic activity of the contacted histone deacetylase identifies the candidate compound as a histone deacetylase protein inhibitor that inhibits a histone deacetylase that is involved in induction of cell differentiation. In certain preferred embodiments, the histone deacetylase protein inhibitor interacts with and reduces the enzymatic activity of fewer than all histone deacetylases.

In an eighth aspect, the invention provides a histone deacetylase protein inhibitor identified by the method of the fifth or the seventh aspects of the invention. Preferably, the histone deacetylase protein inhibitor is substantially pure.

Substantially purified proteins can be achieve by any standard method including, without limitation, expression of recombinant protein, affinity chromatography, antibody-based affinity purification, and high performance liquid chromatography (HPLC; see, e.g., Fisher (1980) Laboratory Techniques in Biochemistry and Molecular Biology, Work and Burdon (eds.), Elsevier). Preferably, a substantially purified protein is at least 80%, by weight, pure in that it is free from other proteins or naturally-occurring organic molecules. More preferably, a substantially purified protein is at least 90% pure, by weight. Most preferably, a substantially purified protein is at least 95% pure, by weight.

In a ninth aspect, the invention provides a method for inhibiting cell proliferation in a cell comprising contacting a cell with at least two of the reagents selected from the group consisting of an antisense oligonucleotide that inhibits a histone deacetylase, a histone deacetylase protein inhibitor, an antisense oligonucleotide that inhibits a DNA methyltransferase, and a DNA methyltransferase protein inhibitor. In one embodiment, the inhibition of cell growth of the contacted cell is greater than the inhibition of cell growth of a cell contacted with only one of the reagents. In certain preferred embodiments, each of the reagents selected from the group is substantially pure. In preferred embodiments, the cell is a neoplastic cell. In yet additional preferred embodiments, the reagents selected from the group are operably associated.

Antisense oligonucleotides that inhibit DNA methyltransferase are described in Szyf and von Hofe, U.S. Pat. No. 5,578,716, the entire contents of which are incorporated by reference. DNA methyltransferase protein inhibitors include, without limitation, 5-aza-2′-deoxycytidine (5-aza-dC), 5-fluoro-2′-deoxycytidine, 5-aza-cytidine (5-aza-C), or 5,6-dihydro-5-aza-cytidine.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the appended claims.

### EXAMPLE 1

### Screening of Antisense Oligonucleotides

To identify which antisense oligonucleotides were most effective at inhibiting a specific histone deacetylase, a number of oligonucleotides were generated based on the sequences provided in GenBank Accession Number U50079 for HDAC-1 and GenBank Accession Number U31814 for HDAC-2. Some of the oligonucleotides screened were described in Table 2 and Table 3 of Besterman et al., U.S. patent application serial No. 60/104,804, filed Oct. 19, 1998, the entire disclosure of which is hereby incorporated by reference.

In addition, oligonucleotides were generated which were complementary to both HDAC-1 and HDAC-2.

To screen these oligonucleotides for an ability to inhibit the targeted histone deacetylase, a Northern blotting analysis was first performed. To do this, T24 human bladder carcinoma cells (commercially available from the American Type Culture Collection (ATCC), Manassas, Va.) were grown under suggested conditions. Before addition of oligonucleotides, cells were washed with PBS (phosphate buffered saline). Next, lipofectin transfection reagent (Gibco-BRL Mississauga, Ontario), at a concentration of 6.25 μg/ml, was added to serum free OPTIMEM medium (GIBCO/BRL), which was then added to the cells. Oligonucleotides to be screened were then added to different wells of cells (i.e., one oligonucleotide per well of cells). The same concentration of oligonucleotide (e.g., 50 nM) was used per well of cells. The cells were allowed to incubate with lipofectin and oligonucleotide for 4 hours at 37° C. in a cell culture incubator. The cells were then washed with PBS and returned to full serum-containing medium. Twenty-four hours later, the cells were harvested for determination of HDAC mRNA levels by Northern blotting analysis.

For determination of mRNA levels by Northern blot, total RNA was prepared from cells by the guanidinium isothiocyanate standard procedure (see, e.g., Ausubel et al., Current Protocols in Molecular Biology, John Wiley & Sons, New York, N.Y., 1994), with the exception of an additional precipitation step in 2 M LiCl overnight at 4° C. to purify RNA from cellular DNA contamination. Northern blotting analysis was performed according to standard protocols. Probes for HDAC-1 and HDAC-2 were full length cDNA clones generated by PCR amplification from the known sequences for each (e.g., GenBank Accession Nos. U50079 and U31814, respectively). These probes were radiolabelled with ³²P-ATP. Northern blots were scanned and quantified using Alpha Imager (Alpha Innovotech).

The oligonucleotides which showed an ability to reduce the mRNA expression of a targeted histone deacetylase (i.e., were able to inhibit transcription of the histone deacetylase mRNA) were next screened for an ability to inhibit expression of the targeted histone deacetylase protein. To do this, T24 cells were transfected with oligonucleotide using lipofectin as described above. Twenty-four hours later, the cells were lysed according to standard procedures. The whole cell extracts (50 μg) were resolved on 7-15% gradient SDS/PAGE, transferred to PVDF membrane (Amersham, Arlington Heights, Ill.), and subjected to Western blotting analysis with rabbit polyclonal HDAC1- and HDAC-2 specific antibodies (1:500, Santa Cruz Biotech., Santa Cruz, Calif.) were used. Detection was accomplished with a secondary anti-rabbit IgG-HR peroxidase antibody and an enhanced chemiluminescence detection kit (Amersham) accordingly to manufacturer's instructions.

Based on our results, the following antisense oligonucleotides were identified as being most effective at inhibiting the expression of targeted histone deacetylase as determined by both mRNA and protein expression blotting analysis. These oliognucleotides are as follows:

For inhibition of HDAC-1, Oligonucleotide No. MG2608 having the sequence:

5′-GAA ACG TGA GGG ACT CAG CA-3′ (SEQ ID NO: 10).

For inhibition of both HDAC-1 and HDAC-2, Oligonucleotide No. MG2610 is a 25/25/25/25 mixture of four oligonucleotides having the sequences:

(SEQ ID NO:11)
5′-CAG CAA ATT ATG GGT CAT GCG GAU UC-3′;
(SEQ ID NO:12)
5′-CAG CAA GTT ATG AGT CAT GCG GAU UC-3′;
(SEQ ID NO:13)
5′-CAG CAA ATT ATG AGT CAT GCG GAU UC-3′;
(SEQ ID NO:14)
5′-CAG CAA GTT ATG GGT CAT GCG GAU UC-3′;

For inhibition of HDAC-2, Table I shows the antisense oligonucleotides found to be most effective:

TABLE I

| | | | |
|---|---|---|---|
| Oligonucleotide | | | |
| No. | Sequence | SEQ ID NO | Target |

| | | | |
|---|---|---|---|
| MG2628 | 5′-UGC UGC TGC TGC TGC TGC CG-3′ | 15 | 121-141 |
| MG2633 | 5′-CCU CCT GCT GCT GCT GCU GC-3′ | 16 | 132-152 |
| MG2635 | 5′-GGU UCC TTT GGT ATC TGU UU-3′ | 17 | 1605-1625 |
| MG2636 | 5′-CUC CTT GAC TGT ACG CCA UG-3′ | 18 | 1-20 |

(***) target reference numbering is in accordance with HDAC-2, GenBank Accession Number U31814.

To evaluate the specificity of the second generation histone deacetylase antisense oligonucleotides, mismatch control oligonucleotides of HDAC-1 (MG2608) and HDAC-1/2 (MG2610) were generated. These mismatch control oligonucleotides were generated by substituting bases, primarily in the four 5′ and 3′ nucleotides, where the highest affinity with the targeted histone deacetylase-encoding nucleic acid occurs.

HDAC-1 MISMATCH CONTROL (MG2609), has the sequence:

5′-CAA UCG TCA GAG ACT CCG AA-3′ (SEQ ID NO: 19).

HDAC-1/2 MISMATCH CONTROL (MG2637), has a 225/25/25/25 mixture of four oligonucleotides having the sequences:

(SEQ ID NO:20)
5′-AAG GAA GTC ATG AAT GAT GCC CAU UG-3′;
(SEQ ID NO:21)
5′-AAG GAA ATC ATG GAT GAT GCC CAU UG-3′;
(SEQ ID NO:22)
5′-AAG GAA GTC ATG GAT GAT GCC CAU UG-3′;
(SEQ ID NO:23)
5′-AAG GAA ATC ATG AAT GAT GCC CAU UG-3′.

These oligonucleotides (i.e., having SEQ ID NOs: 10-23) were second generation oligonucleotides (i.e., 4×4 hybrids). That is, oligonucleotides having SEQ ID NOs: 10-23 were chemically modified as follows: A equals 2′-deoxyriboadenosine; C equals 2′-deoxyribocytidine; G equals 2′-deoxyriboguanosine; T equals 2′-deoxyribothymidine; A equals riboadenosine; U equals uridine; C equals ribocytidine; and G equals riboguanosine. The underlined bases were 2′-methoxyribose substituted nucleotides. Non-underhned bases indicate deoxyribose nucleosides. The backbone of each oligonucleotide consisted of a phosphorothioate linkage between adjoining nucleotides.

A number of oligonucleotides are next generated which are complementary to HDAC-3, HDAC-4, HDAC-5, HDAC-C, HDAC-D, and HDAC-E. These oligonucleotides are based on the known nucleic acid sequences of these histone deacetylases (see, e.g., GenBank Accession No. U75697 for HDAC-3). Antisense oligonucleotides specific for one of these histone deacetylases are screened for efficacy at inhibiting expression of mRNA and protein as described above for HDAC-1, HDAC-1/2, and HDAC-2. In addition, antisense oligonucleotides that inhibit more than one histone deacetylase (e.g., HDAC-1/3/C-specific) are also generated by mixing antisense oligonucleotides specific for each histone deacetylase and screened for efficacy.

### EXAMPLE 2

### Inhibition of Histone Deacetylase mRNA Expression With Antisense Oligonucleotides

To determine the specificity and dose requirements of the antisense oligonucleotides specific for histone deacetylase-encoding nucleic acid, the dose dependent inhibition of these oligonucleotides on histone deacetylase mRNA expression was examined.

To do this, T24 cells were transfected using lipofectin (as described in Example 1) using 10, 25, 50, or 100 nM oligonucleotide. The cells were harvested twenty-four hours following transfection, RNA prepared, and Northern blotting analysis performed as described in Example 1 using radiolabelled HDAC-1 and HDAC-2 cDNA as probe.

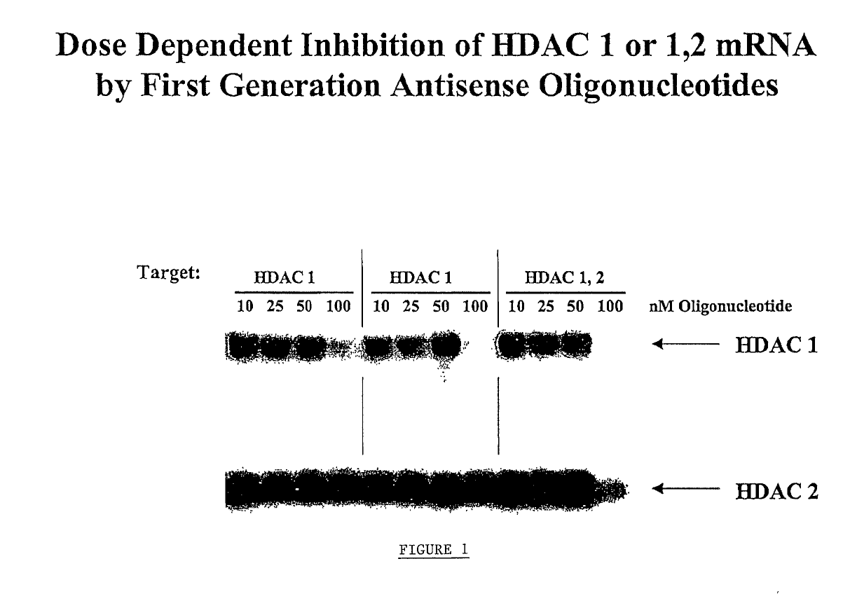

FIG. 1 shows the dose dependent inhibition of HDAC-1 mRNA expression by both HDAC-1 and HDAC-1/2 antisense oligonucleotides at 50-100 nM. Conversely, HDAC-2 mRNA expression was inhibited by only the HDAC-1/2 antisense oligonucleotide (MG2610) at 50-100 nM, while the HDAC-1 antisense oligonucleotide (MG2608) had no effect. The oligonucleotides used in the experiment, the results of which are shown in FIG. 1, were first generation oligonucleotides (i.e., were not chemically modified). The oligonucleotides used to obtain the results shown in FIG. 1 had sequences of SEQ ID NOs: 1-5.

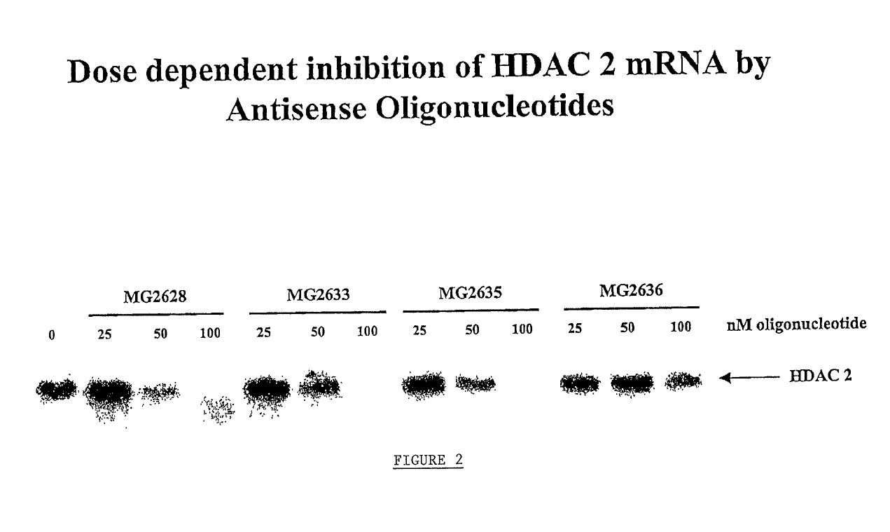

FIG. 2 shows the dose-dependent inhibition of HDAC-2 mRNA by HDAC-2 antisense oligonucleotide. All four HDAC-2 antisense oligonucleotide (MG2628, MG2633, MG2635, and MG2636) were able to reduce the level of HDAC-2 mRNA expression at 50-100 nM. MG2628 appeared particularly efficacious at reducing HDAC-2 mRNA expression in this experiment.

These data demonstrated that by targeting histone deacetylase at the nucleic acid level with antisense oligonucleotide, a reduction in mRNA expression could be achieved 24 hours following exposure to the oligonucleotide.

### EXAMPLE 3

### Inhibition of Histone Deacetylase Protein Expression With Second Generation Antisense Oligonucleotides

To determine the ability of histone deacetylase antisense oligonucleotides to inhibit protein expression, second generation versions of the HDAC-1, HDAC-1/2, and HDAC-2 antisense oligonucleotides were generated. Each of these second generation antisense oligonucleotides had a backbone consisting of a phosphorothioate linkage between each adjoining nucleotide. Moreover, the four terminal nucleotide residues at both the 5′ and 3′ ends of the olgonucleotide had sugar residues comprising a 2′-O-methyl group. This modification to the terminal nucleotide residues served to increase binding affinity of the oligonucleotide to the targeted nucleic acid, and to increase the stability of the oligonucleotide by inhibiting nuclease susceptibility.

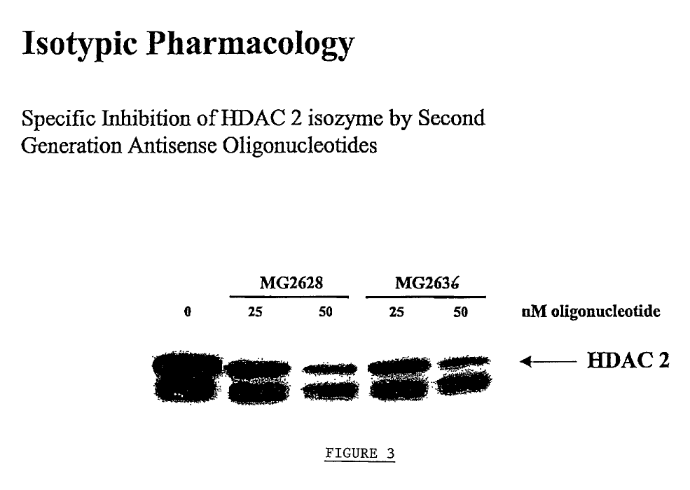

FIG. 3 shows the ability of second generation HDAC-2 antisense oligonucleotides to inhibit HDAC-2 protein expression. T24 cells were transfected with 0, 25, or 50 nM MG2628 or MG2636 using lipofectin, as described in Example 1. Twenty-four hours later, the cells were transfected a second time with the same amount of the same oligonucleotide. Twenty-four hours after this (i.e., 48 hours after the first transfection), cellular proteins were prepared, resolved on 7-15% gradient SDS-PAGE, and subjected to Western blotting analysis as described in Example 1 with rabbit polyclonal HDAC2 specific antibody (1:500, Santa Cruz Biotech). Following blotting with the secondary anti-rabbit IgG-HR peroxidase antibody and visualization with the enhanced chemiluminescence detection kit (Amersham), the blot was stripped and re-probed with an antibody specific to actin to verify equal loading of all wells (data not shown).

As can be seen in FIG. 3, 50 μM of second generation MG2628 or MG2836 was able to inhibit HDAC-2 protein expression.

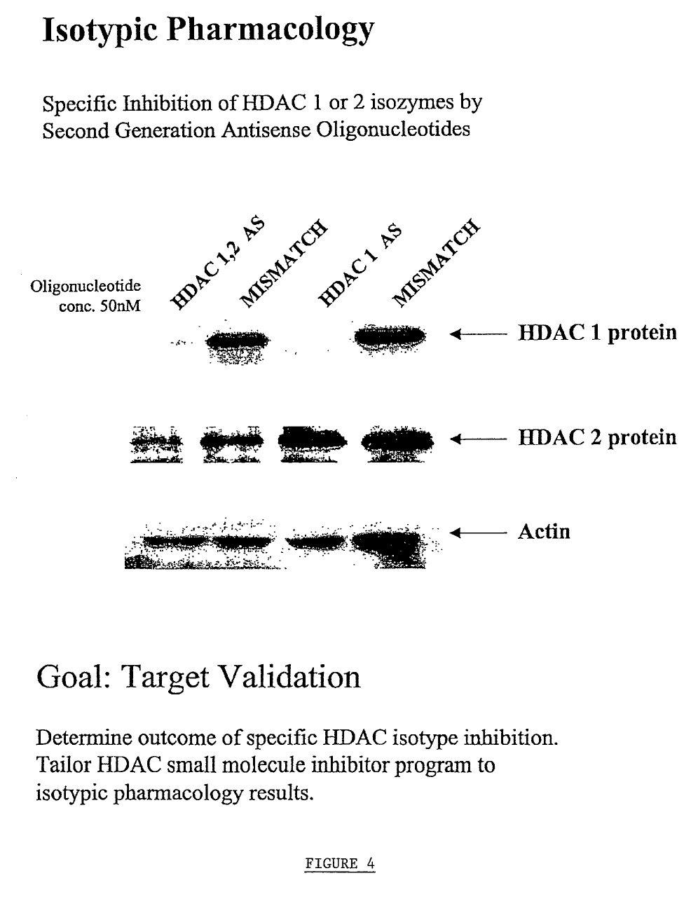

FIG. 4 shows the specific ability of the HDAC-1/2 and HDAC-1 antisense oligonucleotides to inhibit protein expression of both HDAC-1 and HDAC-2 or HDAC-1, respectively, when compared to the mismatch controls. T24 cells were transfected twice as described above with 50 nM oligonucleotide. Cell lysates were prepared twenty-four hours following the second transfection, resolved on 7-15% gradient SDS-PAGE, and transferred to PVDF membrane. The PVDF membrane blot was first blotted with anti-HDAC-1 antibody. Following detection with horseradish peroxidase-labelled secondary antibody and enhanced chemiluminescence, the blot was stripped, and re-probed with anti-HDAC-2 antibody. Following detection, the blot was stripped for a second time and re-probed with an actin-specific antibody to verify equal protein loading in the lanes.

As can be seen in FIG. 4, both HDAC-1 and HDAC-1/2 mismatch control oligonucleotides failed to inhibit HDAC-1 or HDAC-1 and HDAC-2 protein expression, respectively. Conversely, HDAC-1 antisense oligonucleotide effectively reduced expression of HDAC-1 protein, and HDAC-1/2 antisense oligonucleotide reduced protein expression of both HDAC-1 and HDAC-2.

### EXAMPLE 4

### Identification of A Histone Deacetylase Involved in Induction of Cell Proliferation

Antisense oligonucleotides that inhibit expression of different histone deacetylases, according to the invention, are screened to identify a histone deacetylase that induces cell proliferation in cultured cells.

To identify a histone deacetylase that induces normal (i.e., non-neoplastic) cell division, cultured normal human fibroblast cells are transfected with an antisense oligonucleotide that inhibits the expression of a histone deacetylase. While any standard transfection protocol may be employed, including, without limitation, CaPO₄ precipitation, electroporation, DEAE-dextran), transfection using the lipofectin transfection reagent (Gibco-BRL) is preferred. Following transfection with lipofectin and a histone deacetylase antisense oligonucleotide, cells are harvested by trypsinization at various time points, and counted using a hemacytometer or a Coulter Cell Counter. Mock transfected control cells (i.e., treated with lipofectin plus a control, non-specific oligonucleotide) are also harvested and counted. Both the antisense oligonucleotide- and mock-transfected cells are also visually inspected under a microscope for any phenotypic changes (e.g., induction of apoptosis). An antisense oligonucleotide that inhibits the expression of a histone deacetylase that is found to inhibit cell proliferation when transfected into a normal cell identifies a histone deacetylase that is involved in induction of cell proliferation in normal cells.

To identify a histone deacetylase that induces neoplastic cell proliferation, T24 bladder carcinoma cells are transfected with histone deacetylase antisense oligonucleotides according to the invention and their growth pattern is observed and compared to that of untransfected control cells. For this purpose, one day before transfection, T24 cells (ATCC No. HTB-4) are plated onto 10 cm plates at 4×10⁵ cells/dish. At the time of transfection, cells are washed with phosphate buffered saline (PBS) and 5 ml of Opti-MEM media (Gibco-BRL, Mississauga, Ontario) containing 6.25 μg/ml lipofectin transfection reagent is added. The antisense oligonucleotides to be tested are diluted to the desired concentration from a 0.1 mM stock solution in the transfection media. After a four-hour incubation at 37° C. in a 5% CO₂ incubator, the plates are washed with PBS and 10 ml of fresh cell culture media is added. T24 cells are transfected for a total of three days and split every other day to ensure optimal transfection conditions. At various time points, cells are harvested by trypsinization and pelleted by centrifugation at 1100 rpm and 4° C. for five minutes. The cells are resuspended in PBS and counted on a Coulter Particle Counter to determine the total cell number. Mock-transfected T24 cells (transfected with lipofectin and a control oligonucleotide) are similarly grown, harvested, and counted. An antisense oligonucleotide that inhibits the expression of a histone deacetylase that is found to inhibit cell proliferation when transfected into a neoplastic cell identifies a histone deacetylase that is involved in induction of cell proliferation in neoplastic cells.

By screening a number of different histone deacetylase antisense oligonucleotides in normal and neoplastic cells, a histone deacetylase that is involved in induction of cell proliferation may be readily identified. Most preferably, a histone deacetylase antisense oligonucleotide of the invention is one that inhibits cell proliferation of neoplastic cells, but does not inhibit cell proliferation in normal cells.

### EXAMPLE 5

### A Histone Deacetylase Protein Inhibitor that Interacts With and Reduces the Enzymatic Activity of A Histone Deacetylase Involved in the Induction of Cell Proliferation

A histone deacetylase that is identified as being involved in the induction of cell proliferation (identified, for example, in the methods of Example 4), is used as a target for candidate compounds designed to interact with and inhibit its enzymatic activity. As a positive control, FR901228 (available from Fujisawa Pharmaceuticals), is used.

Candidate compounds can be derived from any source and may be naturally-occurring or synthetic, or may have naturally-occurring and synthetic components. Candidate compounds may also be designed to chemically resemble any of the known histone deacetylase protein inhibitors, including, without limitation, trichostatin A, trichostatin C, trapoxin, depudecin, suberoylanilide hydroxamic acid (SAHA), FR901228, and butyrate.

Once candidate compounds are identified, a pool of such compounds may be added to a histone deacetylase. Such a histone deacetylase is preferably one that is identified using the antisense oligonucleotides of the invention as a histone deacetylase involved in induction of cell proliferation. The histone deacetylase may be purified, for example, by using antibodies specific to that particular histone deacetylase (e.g., anti-HDAC-1 antibody commercially available from Santa Cruz Biotech.) or by recombinant production of the histone deacetylase in prokaryotic or eukaryotic cells. The histone deacetylase may also be present in a cell which normally expresses the histone deacetylase.

Pools of candidate compounds are added to the histone deacetylase, and the enzymatic activity of the histone deacetylase is measured. A pool of candidate compounds showing such a histone deacetylase inhibiting activity is sub-divided, and the subdivisions tested until one candidate compound is isolated having a histone deacetylase inhibiting activity. It will be understood that once a pool of candidate compounds is identified as having an ability to inhibit histone deacetylase enzymatic activity, the pool may be screened via various methods to ascertain the presence within the pool or one or more histone deacetylase protein inhibitor compounds. For example, if the pool is initially screened in a cell having a histone deacetylase, the pool may be subsequently screened on purified histone deacetylase.

Preferably, the candidate compound(s) found to be a histone deacetylase protein inhibitor inhibits the activity of fewer than all histone deacetylases. More preferably, such a candidate compound inhibits only those histone deacetylases that are involved in the induction of cell proliferation. Even more preferably, the candidate compound that is identified as a histone deacetylase protein inhibitor is one that inhibits only one histone deacetylase, where that one histone deacetylase is involved in the induction of cell proliferation. Most preferably, the candidate compound that is identified as a histone deacetylase protein inhibitor is one that inhibits only one histone deacetylase, where that one histone deacetylase is involved in the induction of cell proliferation in neoplastic cells, but is not involved in the induction of cell proliferation in normal cells.

In another method to identify a candidate compound that is a histone deacetylase protein inhibitor, purified histone deacetylase is allowed to adhere to the bottom of wells in a 96-well microtiter plate. Candidate compounds (or pools thereof) are then added to the plate, where each candidate compound has been modified with the covalent attachment of a detectable marker (e.g., a biotin label). Binding of the candidate compound to the plate-bound histone deacetylase is detected via addition of a secondary reagent that binds to the detectable marker (e.g., a streptavidin-labelled fluorophore), and subsequent analysis of the plate on a micro-titer plate reader. Candidate compounds thus identified which interact with purified histone deacetylase are then screened for an ability to inhibit the enzymatic activity of the histone deacetylase.

### EXAMPLE 6

### Anti-Neoplastic Effect of Histone Deacetylase Antisense Oligonucleotide on Tumor Cells in Vivo

The purpose of this example is to illustrate the ability of the histone deacetylase antisense oligonucleotide of the invention to treat diseases responsive to histone deacetylase inhibition in animals, particularly mammals. This example further provides evidence of the ability of the methods and compositions of the invention to inhibit tumor growth in domesticated mammal. Eight to ten week old female BALB/c nude mice (Taconic Labs, Great Barrington, N.Y.) are injected subcutaneously in the flank area with 2×10⁶ preconditioned A549 human lung carcinoma cells. Preconditioning of these cells is done by a minimum of three consecutive tumor transplantations in the same strain of nude mice. Subsequently, tumor fragments of approximately 30 mgs are excised and implanted subcutaneously in mice, in the left flank area under Forene anesthesia (Abbott Labs., Geneva, Switzerland). When the tumors reaches a mean volume of 100 mm³, the mice are treated intravenously, by daily bolous infusion into the tail vein, with oligonucleotide saline preparations containing 0.1-6 mg/kg of antisense oligonucleotide (Sigma, St. Louis, Mo.). The optimal final concentration of the oligonucleotide is established by dose response experiments according to standard protocols. Tumor volume is calculated according to standard methods every second day post infusion (e.g., Meyer et al., Int. J. Cancer 43:851-856 (1989)). Treatment with the oligonucleotides according to the invention causes a significant reduction in tumor weight and volume relative to controls treated with saline only (i.e., no oligonucleotide) or controls treated with saline plus a control, non-specific oligonucleotide. In addition, the activity of histone deacetylase when measured is expected to be significantly reduced relative to saline treated controls.

### EXAMPLE 7

### Synergistic Anti-Neoplastic Effect of Histone Deacetylase Antisense Oligonucleotide and Histone Deacetylase Protein Inhibitor on Tumor Cells in Vivo

The purpose of this example is to illustrate the ability of the histone deacetylase antisense oligonucleotide and the histone deacetylase protein inhibitor of the invention to inhibit tumor growth in a mammal. As described in Example 6, mice bearing implanted A549 tumors (mean volume 100 mm³) are treated daily with saline preparations containing from about 0.1 mg to about 30 mg per kg body weight of histone deacetylase antisense oligonucleotide. A second group of mice is treated daily with pharmaceutically acceptable preparations containing from about 0.01 mg to about 5 mg per kg body weight of histone deacetylase protein inhibitor. Some mice receive both the antisense oligonucleotide and the histone deacetylase protein inhibitor. Of these mice, one group may receive the antisense oligonucleotide and the histone deacetylase protein inhibitor simultaneously intravenously via the tail vein. Another group may receive the antisense oligonucleotide via the tail vein, and the histone deacetylase protein inhibitor subcutaneously. Yet another group may receive both the antisense oligonucleotide and the histone deacetylase protein inhibitor simultaneously via a subcutaneous injection. Control groups of mice are similarly established which receive no treatment (e.g., saline only), a mismatch antisense oligonucleotide only, a control compound that does not inhibit histone deacetylase activity, and mismatch antisense oligonucleotide with control compound.

Tumor volume is measured with calipers. Treatment with the antisense oligonucleotide plus the histone deacetylase protein inhibitor according to the invention causes a significant reduction in tumor weight and volume relative to controls. Preferably, the antisense oligonucleotide and the histone deacetylase protein inhibitor inhibit the expression and activity of the same histone deacetylase.

What is claimed is:

1. An antisense oligonucleotide that inhibits the expression of a histone deacetylase.

2. The antisense oligonucleotide of claim 1, wherein the histone deacetylase is selected from the group consisting of HDAC-1, HDAC-2, HDAC-3, HDAC-4, HDAC-5, HDAC-C, HDAC-D, and HDAC-E.

3. The antisense oligonucleotide of claim 1, wherein the oligonucleotide inhibits more than one histone deacetylase.

4. The antisense oligonucleotide of claim 3, wherein the oligonucleotide inhibits all histone deacetylases.

5. The antisense oligonucleotide of claim 1, wherein the oligonucleotide inhibits transcription of a nucleic acid molecule encoding the histone deacetylase.

6. The oligonucleotide of claim 5, wherein the nucleic acid molecule is selected from the group consisting of genomic DNA, cDNA, and RNA.

7. The antisense oligonucleotide of claim 1, wherein the oligonucleotide inhibits translation of the histone deacetylase.

8. The antisense oligonucleotide of claim 1, wherein the oligonucleotide has at least one internucleotide linkage selected from the group consisting of phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages.

9. The antisense oligonucleotide of claim 1, wherein the oligonucleotide is a chimeric oligonucleotide or a hybrid oligonucleotide.

10. The antisense oligonucleotide of claim 1, wherein the oligonucleotide comprises a ribonucleotide or 2′-O-substituted ribonucleotide region and a deoxyribonucleotide region.

11. A method for inhibiting a histone deacetylase in a cell comprising contacting the cell with the antisense oligonucleotide of claim 1.

12. The method of claim 11, wherein cell proliferation is inhibited in the contacted cell.

13. The method of claim 11, wherein the cell is a neoplastic cell.

14. The method of claim 13, wherein neoplastic cell is in an animal.

15. The method of claim 14, wherein the neoplastic cell is in a neoplastic growth.

16. The method of claim 11 further comprising contacting the cell with a histone deacetylase protein inhibitor that interacts with and reduces the enzymatic activity of the histone deacetylase.

17. The method of claim 16, wherein the histone deacetylase protein inhibitor is operably associated with the antisense oligonucleotide.

18. A method for inhibiting neoplastic growth in an animal comprising administering to an animal having at least one neoplastic cell present in its body a therapeutically effective amount of the antisense oligonucleotide of claim 1 with a pharmaceutically acceptable carrier for therapeutically effective period of time.

19. The method of claim 18, wherein the animal is a mammal.

20. The method of claim 19, wherein the mammal is a human.

21. The method of claim 18 further comprising administering to the animal a therapeutically effective amount of a histone deacetylase protein inhibitor that interacts with and reduces the enzymatic activity of the histone deacetylase with a pharmaceutically acceptable carrier for a therapeutically effective period of time.

22. The method of claim 21, wherein the histone deacetylase protein inhibitor is operably associated with the antisense oligonucleotide.

23. A method for identifying a histone deacetylase that is involved in the induction of cell proliferation comprising contacting a cell with an antisense oligonucleotide that inhibits the expression of a histone deacetylase, wherein inhibition of cell proliferation in the contacted cell identifies the histone deacetylase as a histone deacetylase that is involved in the induction of cell proliferation.

24. The method of claim 23, wherein the cell is a neoplastic cell and the induction of cell proliferation is tumorigenesis.

25. The method of claim 23, wherein the histone deacetylase is selected from the group consisting of HDAC-1, HDAC-2, HDAC-3, HDAC-4, HDAC-5, HDAC-C, HDAC-D, and HDAC-E.

26. A method for identifying a histone deacetylase protein inhibitor that inhibits a histone deacetylase that is involved in the induction of cell proliferation comprising contacting a histone deacetylase identified by the method of claim 23 with a candidate compound and measuring the enzymatic activity of the contacted histone deacetylase, wherein a reduction in the enzymatic activity of the contacted histone deacetylase identifies the candidate compound as a histone deacetylase protein inhibitor that inhibits a histone deacetylase that is involved in the induction of cell proliferation.

27. The method of claim 26, wherein the histone deacetylase protein inhibitor interacts with and reduces the enzymatic activity of fewer than all histone deacetylases.

28. A method for identifying a histone deacetylase that is involved in the induction of cell differentiation comprising contacting a cell with an antisense oligonucleotide that inhibits the expression of a histone deacetylase, wherein induction of differentiation in the contacted cell identifies the histone deacetylase as a histone deacetylase that is involved in the induction of cell differentiation.

29. The method of claim 28, wherein the cell is a neoplastic cell.

30. The method of claim 28, wherein the histone deacetylase is selected from the group consisting of HDAC-1, HDAC-2, HDAC-3, HDAC-4, HDAC-5, HDAC-C, HDAC-D, and HDAC-E.

31. A method for identifying a histone deacetylase protein inhibitor that inhibits a histone deacetylase that is involved in the induction of cell differentiation comprising contacting a histone deacetylase identified by the method of claim 28 with a candidate compound and measuring the enzymatic activity of the contacted histone deacetylase, wherein a reduction in the enzymatic activity of the contacted histone deacetylase identifies the candidate compound as a histone deacetylase protein inhibitor that inhibits a histone deacetylase that is involved in the induction of cell differentiation.

32. The method of claim 31, wherein the histone deacetylase protein inhibitor interacts with and reduces the enzymatic activity of fewer than all histone deacetylases.

33. A histone deacetylase protein inhibitor identified by the method of claim 26 or 31.

34. The histone deacetylase protein inhibitor is substantially pure.

35. A method for inhibiting cell proliferation in a cell comprising contacting a cell with at least two of the reagents selected from the group consisting of an antisense oligonucleotide that inhibits a histone deacetylase, a histone deacetylase protein inhibitor, an antisense oligonucleotide that inhibits a DNA methyltransferase, and a DNA methyltransferase protein inhibitor.

36. The method of claim 35, wherein the inhibition of cell growth of the contacted cell is greater than the inhibition of cell growth of a cell contacted with only one of the reagents.

37. The method of claim 35, wherein the each of the reagents selected from the group is substantially pure.

38. The method of claim 35, wherein the cell is a neoplastic cell.

39. The method of claim 35, wherein the reagents selected from the group are operably associated.
